(19) United States
(12) Patent Application Publication
(10) Pub. No.: US 2025/0002524 A1
(43) Pub. Date: Jan. 2, 2025

(54) CAP ANALOGS HAVING AN ACYCLIC LINKER TO THE GUANINE DERIVATIVE NUCLEOBASE

(71) Applicant: CureVac SE, Tübingen (DE)

(72) Inventor: Rainer Joachim SCHWARZ, Tübingen (DE)

(73) Assignee: CureVac SE, Tübingen (DE)

(21) Appl. No.: 18/293,593

(22) PCT Filed: Jul. 29, 2022
(86) PCT No.: PCT/EP2022/071478

§ 371 Date: Jan. 30, 2024

(30) Foreign Application Priority Data
Jul. 30, 2021 (WO) PCT/EP2021/071432

(51) Int. Cl. C07H 21/02; A61K 31/7088; C07H 19/20

(52) U.S. Cl. CPC C07H 21/02; A61K 31/7088; C07H 19/20

(57) ABSTRACT

The present invention is inter alia concerned with (A) a compound of formula (I) as defined herein or a salt, stereoisomer, tautomer or deuterated version thereof, (B) a cap analog comprising a 5′ terminal acyclonucleoside, wherein the acyclonucleoside comprises a linear unbranched structure or a linear single-branched structure instead of a ribose, wherein the 5′ terminal acyclonucleoside is optionally deuterated (C) an RNA molecule comprising at least three nucleotides and comprising a 5′ end of formula (III) as defined herein, wherein the 5′ end is optionally deuterated (D) an RNA molecule comprising at least three nucleotides and comprising a 5′ terminal acyclonucleoside, wherein the acyclonucleoside comprises a linear unbranched structure or a linear single-branched structure instead of a ribose, wherein the 5′ terminal acyclonucleoside is optionally deuterated (E) an in vitro method for synthesizing an RNA molecule, (F) the RNA molecule obtained thereby, (G) compositions comprising the RNA molecule, (H) kits comprising the compound of formula (I) or the cap analog, (I) uses as well as (J) methods as outlined herein.

### TECHNICAL FIELD

The present invention relates to a compound of formula (I) as defined herein or a salt, stereoisomer, tautomer or deuterated version thereof. The present invention further relates to a cap analog comprising a 5′ terminal acyclonucleoside, wherein the acyclonucleoside comprises a linear unbranched structure or a linear single-branched structure instead of a ribose, wherein the cap analog is a cap1 analog or a cap2 analog and wherein the 5′ terminal acyclonucleoside is optionally deuterated. The present invention further relates to an RNA molecule comprising at least three nucleotides and comprising a 5′ end of formula (III) as defined herein, wherein the 5′ end is optionally deuterated, and an RNA molecule comprising at least three nucleotides and comprising a 5′ terminal acyclonucleoside, wherein the acyclonucleoside comprises a linear unbranched structure or a linear single-branched structure instead of a ribose, and wherein the 5′ terminal acyclonucleoside is optionally deuterated. Further, the present invention relates to an in vitro method for synthesizing an RNA molecule as well as the RNA molecule obtained thereby. Compositions comprising the RNA molecule, kits comprising the compound of formula (I) or the cap analog, uses as well as methods as outlined in the following are also part of the present invention.

### BACKGROUND OF THE INVENTION

Eukaryotic mRNA has a cap structure at its 5′-terminus, wherein this cap structure consists of 7-methyl guanosine (m⁷G) and a triphosphate bridge (ppp) linking the 5′OH of the m⁷G to the 5′OH of the 5-terminal nucleotide (N). This structure can be referred to as m⁷G(5′)pppN. The cap structure of an mRNA is inter alia implicated in eukaryotic cells in the assembly of the translation initiation complex by binding to the eukaryotic translation initiation factor 4E (eIF-4E). It is therefore essential to maintain a cap structure in mRNAs that are produced in vitro and that are intended to be used in pharmaceutical products. In such products, the mRNAs are translated in and by the cells of the subject to be treated into the encoded peptides or proteins. Typically, such mRNAs are produced in in vitro transcription reactions using a DNA template and a DNA-dependent RNA polymerase, such as in particular T7 or SP6 DNA-dependent RNA polymerase. The capping can either be carried out co-transcriptionally or after the transcription reaction.

For co-transcriptional capping reactions, m⁷G(5′)ppp was found to be used by T7 or SP6 DNA-dependent RNA polymerase in vitro to initiate the transcription reaction. However, m⁷G(5′)ppp has the disadvantage of having to compete with the guanine nucleotide (G) as the initiating nucleophile for transcription elongation such that less than half of the in vitro produced mRNAs have a cap structure at their 5′-terminal if m⁷G(5′)ppp is used.

Dinucleotide-cap analoga have also been developed and described (E. Darzynkiewicz and A. J. Shatkin, Biochemistry 1985, 24, 7, 1701-1707), in particular the cap analog m⁷G(5′)ppp(5′)G. m⁷G(5′)ppp(5′)G has been successfully used in in vitro transcription reactions as initiator of transcription to produce cap structures co-transcriptionally. However, m⁷G(5′)ppp(5′)G has the disadvantage that the 3′-OH group of either the m⁷G or the G moiety can serve as the initiating nucleophile for transcriptional elongation. Accordingly, two different RNAs are produced, namely m⁷G(5′)pppG(pN)n (with the correct orientation of the cap) and G(5′)pppm⁷G(pN)n (with the reverse orientation of the cap), with one third to half of the cap structures oriented in the reverse direction. In order to render the reverse orientation impossible during the in vitro reaction, so-called anti-reverse cap analogs (ARCAs) have been developed, where the 3′-OH group of the m¹G moiety is replaced with hydrogen or OCH₃ (J Stepinski and R E Rhoads; RNA. 2001 October; 7(10): 1486-1495. PMID: 116808539).

Further cap analogs that aim at increasing the binding efficiency towards eIF-4E and the expression level have been developed. Modification sites were the N7-Position of the cap (WO 2016/098028), the ribose of the m⁷G (WO 2017/066797; U.S. Pat. No. 7,074,596), the triphosphate bridge (WO 2009/149253; WO 2017/066781; WO 2017/066791; A. M Rydzik, J. Jemielity, Bioorg Med Chem. 2012; 20(5):1699-710 PMID: 22316555; J. Kowalska and J. Jemielity Nucleic Acids Res. 2014; 42(16):10245-64 PMID: 25150148; B. A Wojtczak, J. Jemielity, J Am Chem Soc. 2018 May 9; 140(18):5987-5999 PMID: 29676910) and the first translated nucleotide (S. Akichika and T. Suzuki Science. 2019 Jan. 11; 363 (6423):eaav0080 PMID: 30467178; M. Kopcial and J. Jemielity, Molecules. 2019 May 17; 24(10):1899 PMID: 31108861).

A general disadvantage of the afore-mentioned analoga is the recognition of these structures by IFIT1 and IFIT3 proteins, resulting in immunostimulation (B. Johnson and G. K. Amarasinghe, 2018 Mar. 20; 48(3):487-499 PMID: 29525521). In order to reduce the immunostimulation, trinucleotide analogs have been developed, which are also suitable for co-transcriptional capping. An example of such analogs is m⁷GpppNmpN, where the 2′-OH group of the first translated nucleotide is methylated (Nm). Such cap analogs show a high capping efficiency and lead to a high expression of the resulting mRNA (WO 2017/053297; P. J Sikorski and J. Jemielity Nucleic Acids Res. 2020 Feb. 28; 48(4):1607-1626 PMID: 31984425).

There remains a need to provide cap analogs that have inter alia a high efficiency as regards the co-transcriptional capping in in vitro transcription reactions and that result in in high expression levels of capped RNAs produced by in vitro reactions using such cap analogs.

### SUMMARY OF THE INVENTION

The inventors solved the above need in that they surprisingly found new cap analogs as described herein.

In the first aspect, the present invention relates to a compound of formula (I):

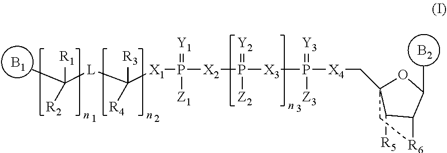

or a salt, stereoisomer, tautomer or deuterated version thereof, wherein
R₅ is

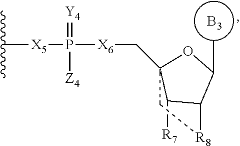

R₇ is OH or

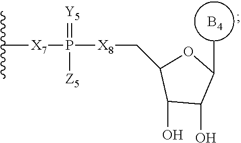

ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂ (halogen),
CH(halogen)₂, and C(halogen)₃ and each of the remaining three of R₁ through R₄ is independently H, OH, SH,
NH₂ or halogen;
n₁ and n₂ are each independently selected from an integer ranging from 0 to 10;
n₃ is selected from 0, 1 or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂,
CH(OH), CH(SH) and CH(halogen);
each of X₁ through X₈ is independently O, S, NH or CH₂;
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) O, wherein the dashed methylene bridge between R₆ being O and the 4′ C is present;
R₈ is (i) selected from the group consisting of H, OH, OC₁-C₃-alkyl, and Opropargyl, wherein the dashed methylene bridge between R₈ and the 4′ C is absent, or (ii) O, wherein the dashed methylene bridge between R₈ being O and the 4′ C is present; and
each of ring B₂ through ring B₄ is independently a nucleobase, a modified nucleobase or a nucleobase analog.

In an embodiment of the first aspect, n₃ is 1.

In an embodiment of the first aspect, R₆ is selected from the group consisting of H, OH, OC₁-C₃-alkyl, and Opropargyl, wherein the dashed methylene bridge between R₆ and the 4′ C is absent.

In an embodiment of the first aspect, R₈ is selected from the group consisting of H, OH, OC₁-C₃-alkyl, and Opropargyl, wherein the dashed methylene bridge between R₈ and the 4′ C is absent.

In yet another embodiment of the first aspect, n₃ is 1; R₆ is selected from the group consisting of H, OH, OC₁-C₃-alkyl, and Opropargyl, wherein the dashed methylene bridge between R₆ and the 4′ C is absent; and R₈ is selected from the group consisting of H, OH, OC₁-C₃-alkyl, and Opropargyl, wherein the dashed methylene bridge between R₈ and the 4′ C is absent.

In an embodiment of the first aspect, B₂ is selected from the group consisting of guanine, a modified guanine, a guanine analog, adenine, a modified adenine, and an adenine analog.

In an embodiment of the first aspect, B₃ is guanine, a modified guanine or a guanine analog.

In an embodiment of the first aspect, X₁ is CH₂ and each of X₂ through X₃ is independently O, S, NH or CH₂.

In an embodiment of embodiment 1 of set (B) of embodiments as listed below herein (i.e. not an embodiment of the first aspect), if both B₂ and B₃ are present, B₂ is selected from the group consisting of guanine, a modified guanine, a guanine analog, adenine, a modified adenine, and an adenine analog; and B₃ is guanine, a modified guanine or a guanine analog.

In yet another embodiment of embodiment 1 of set (B) of embodiments as listed below herein (i.e. not an embodiment of the first aspect), R₅ is OH and R₆ is OH, wherein the dashed methylene bridge between R₆ and the 4′ C is absent. In this embodiment, the compound of the present invention is a dinucleotide-like compound (inter alia since it comprises two nucleobases, namely rings B₁ and B₂), which may also be referred to as having a cap0 structure./being a cap0 analog.

In another embodiment of the first aspect, R₅ is

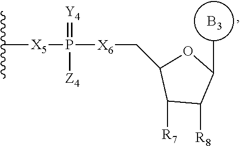

wherein R₇ and R₈ are each OH. In this embodiment, the compound of the present invention is a trinucleotide-like compound (inter alia since it comprises three nucleobases, namely rings B₁ to B₃), which may also be referred to as having a cap1 structure/being a cap1 analog. It can be preferred in this embodiment that R₆ is H or OC₁-C₃-alkyl, wherein it can be especially preferred that R₆ is OCH₃. It can also be preferred in this embodiment that R₆ is O, wherein the dashed methylene bridge between R₆ being O and the 4′ C is present.

When it comes to a cap1 structure, a particular embodiment relates to a compound according to the first aspect, wherein n₁ is 1; n₂ is 2, n₃ is 1; L is O; each of R₁ through R₄ is H; R₅ is

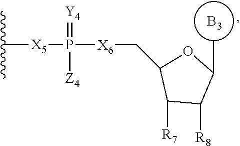

wherein R₇ and R₈ are each OH; R₆ is OCH₃; each of X₁ through X₆ is O, or X₁ is CH₂ and each of X₂ through X₆ is O; each of Z₁ through Z₄ is OH; and each of Y₁ through Y₄ is O. It can further be preferred in this embodiment that B₁ is a modified guanine, in particular N⁷-methylguanine; B₂ is adenine; and B₃ is guanine. An exemplary compound in this respect can in particular be the compound of formula (IV) as shown in the following (in a specific salt form, any other forms and salts are understood to be encompassed as well):

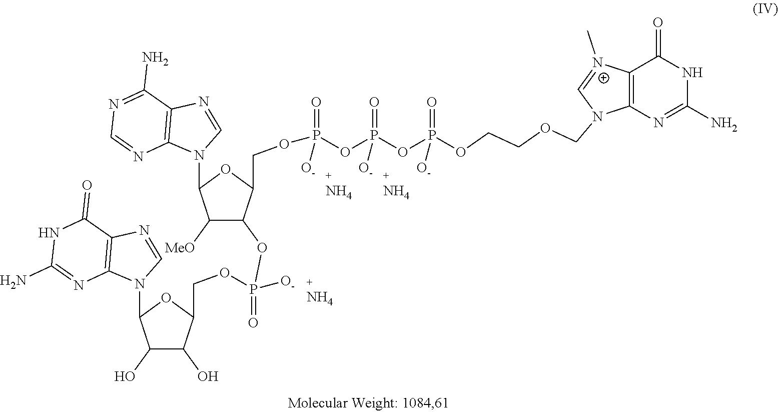

When it comes to a cap1 structure, another particular embodiment relates to a compound according to the first aspect, wherein n₁ is 1; n₂ is 2, n₃ is 1; L is 0; each of R₁ through R₄ is H; R₅ is

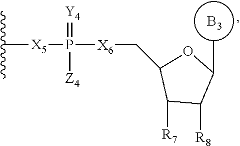

wherein R₇ and R₈ are each OH; R₆ is O, wherein the dashed methylene bridge between R₆ being O and the 4′ C is present; each of X₁ through X₆ is O, or X₁ is CH₂ and each of X₂ through X₆ is 0; each of Z₁ through Z₄ is OH; and each of Y₁ through Y₄ is O. It can further be preferred in this embodiment that B₁ is a modified guanine, in particular N⁷-methylguanine; B₂ is adenine; and B₃ is guanine. An exemplary compound in this respect can in particular be the compound of formula (V) as shown in the following (in a specific salt form, any other forms and salts are understood to be encompassed as well):

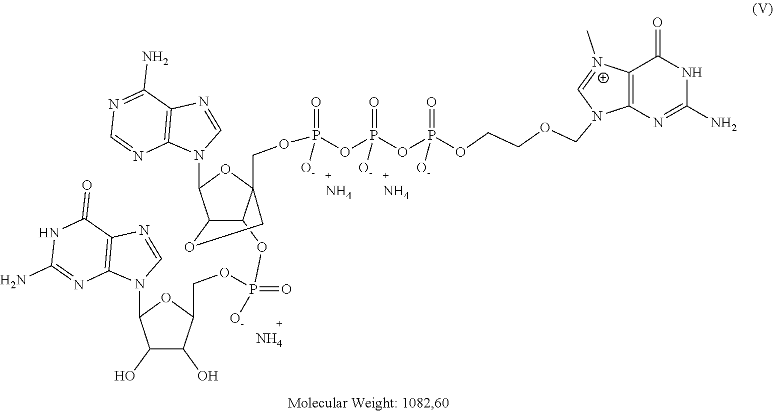

In yet another embodiment of the first aspect, R₅ is

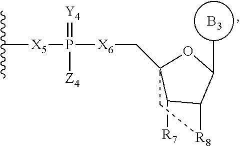

wherein R₇ is

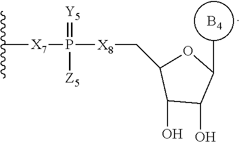

In this embodiment, the compound of the present invention is a tetranucleotide-like compound (inter alia since it comprises four nucleobases, namely rings B₁ to B₄), which may also be referred to as having a cap2 structure/being a cap2 analog. It can be preferred in this embodiment that R₆ is H or OC₁-C₃-alkyl, wherein the dashed methylene bridge between R₆ and the 4′ C is absent, preferably wherein R₆ is OCH₃ (wherein also in this preferred embodiment the dashed methylene bridge between R₆ and the 4′ C is absent); and R₈ is H or OC₁-C₃-alkyl, wherein the dashed methylene bridge between R₈ and the 4′ C is absent, preferably wherein R₈ is OCH₃ (wherein also in this preferred embodiment the dashed methylene bridge between R₆ and the 4′ C is absent). Alternatively, it can be preferred that R₆ is O, wherein the dashed methylene bridge between R₆ being O and the 4′ C is present, and/or that R₈ is O, wherein the dashed methylene bridge between R₈ being O and the 4′ C is present.

In a preferred embodiment of the first aspect, ring B₁ is a modified guanine. It can be especially preferred that ring B₁ is N⁷-methylguanine.

In yet another preferred embodiment of the first aspect, each of X₂ through X₈ is 0. In some embodiments, X₁ is also 0, whereas in other embodiments X₁ is CH₂. Thus, under reference to the examples, it is noted that X₁ is 0 when it comes to the compounds obtained by synthesis routes I and II, whereas X₁ is CH₂ when it comes to the compounds obtained by synthesis route Ill. It is preferred that X₁ is CH₂.

In another preferred embodiment of the first aspect, each of Y₁ through Y₅ is O.

In another preferred embodiment of the first aspect, each of Z₁ through Z₅ is OH.

In yet another preferred embodiment of the first aspect, each of R₁ through R₄ is independently H or OH; or one of R₁ through R₄ is selected from the group consisting of CH₃, CH₂(OH), and CH(OH)₂, and each of the remaining three of R₁ through R₄ is independently H or OH. It can be preferred that one of R₃ through R₄ is selected from the group consisting of CH₃, CH₂(OH), and CH(OH)₂, and each of the remaining three of R₁ through R₄ is independently H or OH.

In another preferred embodiment of the first aspect, each of R₁ through R₃ is H and R₄ is H or OH. It can in other embodiments be preferred that each of R₁ through R₄ is H; or one of R₁ through R₄ is selected from the group consisting of CH₃, CH₂(OH), and CH(OH)₂, and each of the remaining three of R₁ through R₄ is H or OH; and preferably one of R₃ through R₄ is selected from the group consisting of CH₃, CH₂(OH), and CH(OH)₂, and each of the remaining three of R₁ through R₄ is independently H or OH.

In yet another preferred embodiment of the first aspect, n₁ and n₂ are each independently selected from an integer ranging from 0 to 3. It can be preferred that n₁ is selected from 0, 1, 2 or 3; and n₂ is selected from 0, 1 or 2. It can also be preferred that n₁ is 0; and n₂ is selected from 1 or 2. It can also be preferred that n₁ is 1; and n₂ is selected from 1 or 2. Still further, it can be preferred that n₁ is 2; and n₂ is selected from 1 or 2. Also, it can be preferred that n₁ is selected from 1 or 2; and n₂ is 0. It can be preferred that n₁ is selected from 1 or 2; and n₂ is 1. Yet in another preferred embodiment, n₁ is selected from 1 or 2; and n₂ is 2. It can still be preferred that n₁ is 3; and n₂ is 1 or that n₁ is 2; and n₂ is 0.

In another preferred embodiment of the first aspect, L is selected from the group consisting of CH₂, O, S, SO, SO₂ and CH(OH).

In another preferred embodiment of the first aspect, (i) each of R₁ through R₄ is H; (ii) n₁ is selected from 0, 1 or 2; (iii) n₂ is selected from 1 or 2; (iv) L is selected from CH₂ and O; and (v) X₁ is O. This embodiment may in particular refer to compounds prepared by synthesis route I of the present examples. It can be preferred in this embodiment that n₃ is 1; R₆ is selected from the group consisting of H, OH, OC₁-C₃-alkyl, and Opropargyl, wherein the dashed methylene bridge between R₆ and the 4′ C is absent; and Rₐ is selected from the group consisting of H, OH, OC₁-C₃-alkyl, and Opropargyl, wherein the dashed methylene bridge between R₈ and the 4′ C is absent.

In yet another preferred embodiment of the first aspect, (i) each of R₁ through R₃ is H; (ii) R₄ is H or OH; (iii) each of n₁ and n₂ is selected from 1 or 2; (iv) L is selected from CH₂, O and CH(OH); and (v) X₁ is 0. This embodiment may in particular refer to compounds prepared by synthesis route II of the present examples. It can be preferred in this embodiment that n₃ is 1; R₆ is selected from the group consisting of H, OH, OC₁-C₃-alkyl, and Opropargyl, wherein the dashed methylene bridge between R₆ and the 4′ C is absent; and R₈ is selected from the group consisting of H, OH, OC₁-C₃-alkyl, and Opropargyl, wherein the dashed methylene bridge between R₈ and the 4′ C is absent.

In still another preferred embodiment of the first aspect, (i) each of R₁ through R₄ is H; (ii) n₁ is selected from 1, 2 or 3; (iii) n₂ is selected from 0, 1 or 2; (iii) L is selected from S, SO and SO₂; and (iv) X₁ is CH₂. This embodiment may in particular refer to compounds prepared by synthesis route III of the present examples. It can be preferred in this embodiment that n₃ is 1; R₆ is selected from the group consisting of H, OH, OC₁-C₃-alkyl, and Opropargyl, wherein the dashed methylene bridge between R₆ and the 4′ C is absent; and R₈ is selected from the group consisting of H, OH, OC₁-C₃-alkyl, and Opropargyl, wherein the dashed methylene bridge between R₃ and the 4′ C is absent.

In still another preferred embodiment of the first aspect, (i) each of R₁ through R₄ is H; (ii) n₁ is 2; (iii) n₂ is 1; (iii) L is CH₂; and (iv) X₁ is O. It can be preferred in this embodiment that each of X₂ through X₈ is O; each of Y₁ through Y₅ is O; each of Z₁ through Z₅ is OH; and B₁ is N⁷-methylguanine. It can further be preferred in this embodiment that R₆ is OCH₃ and that, optionally, R₈ is OCH₃ (wherein the corresponding dashed methylene bridges are absent).

In still another preferred embodiment of the first aspect, (i) each of R₁ through R₄ is H; (ii) n₁ is 2; (iii) n₂ is 2; (iii) L is O; and (iv) X₁ is O. It can be preferred in this embodiment that each of X₂ through X₈ is O; each of Y₁ through Y₅ is O; each of Z₁ through Z₅ is OH; and B₁ is N⁷-methylguanine. It can further be preferred in this embodiment that R₆ is OCH₃ and that, optionally, R₈ is OCH₃ (wherein the corresponding dashed methylene bridges are absent).

In still another preferred embodiment of the first aspect, (i) each of R₁ through R₄ is H; (ii) n₁ is 2; (iii) n₂ is 1; (iii) L is S; and (iv) X₁ is CH₂. It can be preferred in this embodiment that each of X₂ through X₈ is O; each of Y₁ through Y₅ is O; each of Z₁ through Z₅ is OH; and B₁ is N⁷-methylguanine. It can further be preferred in this embodiment that R₆ is OCH₃ and that, optionally, R₈ is OCH₃ (wherein the corresponding dashed methylene bridges are absent).

In still another preferred embodiment of the first aspect, (i) each of R₁ through R₄ is H; (ii) n₁ is 2; (iii) n₂ is 0; (iii) L is S; and (iv) X₁ is CH₂. It can be preferred in this embodiment that each of X₂ through X₈ is O; each of Y₁ through Y₅ is O; each of Z₁ through Z₅ is OH; and B₁ is N⁷-methylguanine. It can further be preferred in this embodiment that R₆ is OCH₃ and that, optionally, R₈ is OCH₃ (wherein the corresponding dashed methylene bridges are absent).

In the second aspect, the present invention relates to a cap analog comprising a 5′ terminal acyclonucleoside, wherein the acyclonucleoside comprises a linear unbranched structure or a linear single-branched structure instead of a ribose, wherein the cap analog is a cap1 analog or a cap2 analog, and wherein the 5′ terminal acyclonucleoside is optionally deuterated. The cap analog of the second aspect can be characterized in that it is suitable for initiating RNA in vitro transcription.

In an embodiment of the second aspect, the linear unbranched structure has the structure of formula (II):

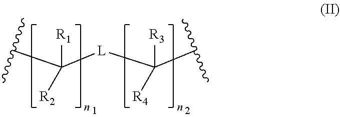

wherein
each of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the
proviso that, if (i) n₁ is 0 and/or (ii) n₂ is 0, L is selected from the group consisting of CH₂, CH(OH), CH(SH) and CH(halogen).

In an embodiment thereof, each of R₁ through R₄ is independently H or OH. In another embodiment thereof, each of R₁ through R₃ is H and R₄ is H or OH. In another embodiment thereof, each of R₁ through R₄ is H.

In another embodiment of the second aspect, the linear single-branched structure has the structure of formula (II):

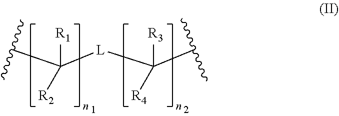

wherein
one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).

In an embodiment thereof, one of R₁ through R₄ is selected from the group consisting of CH₂, CH(OH), and C(OH)₂, and each of the remaining three of R₁ through R₄ is independently H or OH.

In yet another embodiment relating to the above second aspect, n₁ and n₂ are each independently selected from an integer ranging from 0 to 3. It can be preferred that n₁ is selected from 0, 1, 2 or 3; and n₂ is selected from 0, 1 or 2. It can also be preferred that n₁ is 0; and n₂ is selected from 1 or 2. It can also be preferred that n₁ is 1; and n₂ is selected from 1 or 2. Still further, it can be preferred that n₁ is 2; and n₂ is selected from 1 or 2. Also, it can be preferred that n₁ is selected from 1 or 2; and n₂ is 0. It can be preferred that n₁ is selected from 1 or 2; and n₂ is 1. Yet in another preferred embodiment, n₁ is selected from 1 or 2; and n₂ is 2. It can still be preferred that n₁ is 3; and n₂ is 1 or that n₁ is 2; and n₂ is 0.

In another embodiment thereof, L is selected from the group consisting of CH₂, O, S, SO, SO₂ and CH(OH).

In yet another embodiment thereof, each of R₁ through R₄ is independently H or OH; n₁ and n₂ are each independently selected from an integer ranging from 0 to 3; and L is selected from the group consisting of CH₂, O, S, SO, SO₂ and CH(OH).

In another preferred embodiment thereof, (i) each of R₁ through R₄ is H; (ii) n₁ is selected from 0, 1 or 2; (iii) n₂ is selected from 1 or 2; and (iv) L is selected from CH₂ and O.

In yet another preferred embodiment thereof, (i) each of R₁ through R₃ is H; (ii) R₄ is H or OH; (iii) each of n₁ and n₂ is selected from 1 or 2; and (iv) L is selected from CH₂, O and CH(OH).

In still another preferred embodiment thereof, (i) each of R₁ through R₄ is H; (ii) n₁ is selected from 1, 2 or 3; (iii) n₂ is selected from 0, 1 or 2; and (iii) L is selected from S, SO and SO₂.

In yet another embodiment thereof, one of R₁ through R₄ is selected from the group consisting of CH₃, CH₂(OH), and CH(OH)₂, and each of the remaining three of R₁ through R₄ is independently H or OH; n₁ and n₂ are each independently selected from an integer ranging from 0 to 3; and L is selected from the group consisting of CH₂, O, S, SO, SO₂ and CH(OH).

In another preferred embodiment thereof, (i) one of R₁ through R₄ is selected from the group consisting of CH₃, CH₂(OH), and CH(OH)₂, and each of the remaining three of R₁ through R₄ is H; (ii) n₁ is selected from 0, 1 or 2; (iii) n₂ is selected from 1 or 2; and (iv) L is selected from CH₂ and O.

In yet another preferred embodiment thereof, (i) one of R₁ through R₄ is selected from the group consisting of CH₃, CH₂(OH), and CH(OH)₂, and each of the remaining three of R₁ through R₄ is H; preferably one of R₃ through R₄ is selected from the group consisting of CH₃, CH₂(OH), and CH(OH)₂, and each of the remaining three of R₁ through R₄ is H; (ii) each of n₁ and n₂ is selected from 1 or 2; and (iii) L is selected from CH₂, O and CH(OH).

In still another preferred embodiment thereof, (i) one of R₁ through R₄ is CH₃, and each of the remaining three of R₁ through R₄ is H, preferably one of R₃ through R₄ is CH₃, and each of the remaining three of R₁ through R₄ is H; (ii) n₁ is selected from 1, 2 or 3; (iii) n₂ is selected from 0, 1 or 2; and (iii) L is selected from S, SO and SO₂.

In a preferred embodiment of the second aspect, the acyclonucleoside comprises as the nucleobase guanine, a modified guanine or a guanine analog. It can be especially preferred that the acyclonucleoside comprises as the nucleobase a modified guanine, most preferably N⁷-methylguanine.

In the third aspect, the present invention relates to an RNA molecule comprising at least three nucleotides and comprising a 5′ end of formula (III):

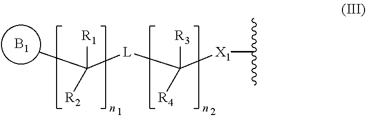

wherein
ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂,
CH(OH), CH(SH) and CH(halogen);
X₁ is O, S, NH or CH₂; and
wherein the 5′ end is optionally deuterated.

In the third aspect, formula (III) corresponds to the cap nucleoside at the 5′ end. This cap nucleoside is typically linked to the remainder of the RNA molecule via a triphosphate bridge, wherein the triphosphate bridge connects X₁ of formula (III) [as indicated in formula (III)] and the 5′ position of the ribose of the first regular nucleotide of the RNA, i.e. the remainder of the RNA molecule.

In an embodiment of the third aspect, each of R₁ through R₄ is independently H or OH. In another embodiment, each of R₁ through R₃ is H and R₄ is H or OH. In another embodiment, each of R₁ through R₄ is H.

In yet another embodiment of the third aspect, n₁ and n₂ are each independently selected from an integer ranging from 0 to 3. It can be preferred that n₁ is selected from 0, 1, 2 or 3; and n₂ is selected from 0, 1 or 2. It can also be preferred that n₁ is 0; and n₂ is selected from 1 or 2. It can also be preferred that n₁ is 1; and n₂ is selected from 1 or 2. Still further, it can be preferred that n₁ is 2; and n₂ is selected from 1 or 2. Also, it can be preferred that n₁ is selected from 1 or 2; and n₂ is 0. It can be preferred that n₁ is selected from 1 or 2; and n₂ is 1. Yet in another preferred embodiment, n₁ is selected from 1 or 2; and n₂ is 2. It can still be preferred that n₁ is 3; and n₂ is 1 or that n₁ is 2; and n₂ is 0.

In another embodiment of the third aspect, L is selected from the group consisting of CH₂, O, S, SO, SO₂ and CH(OH).

In yet another embodiment of the third aspect, each of R₁ through R₄ is independently H or OH; n₁ and n₂ are each independently selected from an integer ranging from 0 to 3; L is selected from the group consisting of CH₂, O, S, SO, SO₂ and CH(OH); and X₁ is O or CH₂.

In another preferred embodiment of the third aspect, (i) each of R₁ through R₄ is H; (ii) n₁ is selected from 0, 1 or 2; (iii) n₂ is selected from 1 or 2; (iv) L is selected from CH₂ and O; and (v) X₁ is O.

In yet another preferred embodiment of the third aspect, (i) each of R₁ through R₃ is H; (ii) R₄ is H or OH; (iii) each of n₁ and n₂ is selected from 1 or 2; (iv) L is selected from CH₂, O and CH(OH); and (v) X₁ is O.

In still another preferred embodiment of the third aspect, (i) each of R₁ through R₄ is H; (ii) n₁ is selected from 1, 2 or 3; (iii) n₂ is selected from 0, 1 or 2; (iii) L is selected from S, SO and SO₂; and (iv) X₁ is CH₂.

In an especially preferred embodiment of the third aspect, ring B₁ is a modified guanine, preferably N⁷-methylguanine.

In another embodiment of the third aspect, the RNA molecule comprises a 5′ end of formula (I):

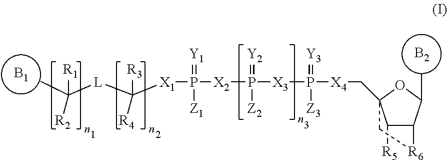

wherein
R₅ is

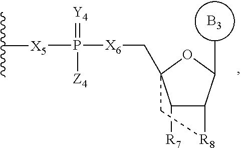

wherein
R₇ is

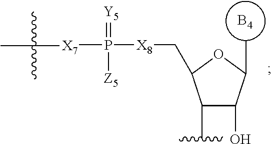

and wherein
n₃ is selected from 0, 1 or 2;
each of X₂ through X₈ is independently O, S, NH or CH_{2;}
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) 0, wherein the dashed methylene bridge between R₆ being O and the 4′ C is present;
R₈ is (i) selected from the group consisting of H, OH, OC₁-C₃-alkyl, and Opropargyl, wherein the dashed methylene bridge between R₈ and the 4′ C is absent, or (ii) 0, wherein the dashed methylene bridge between R₈ being O and the 4′ C is present; and
each of ring B₂ through ring B₄ is independently a nucleobase, a modified nucleobase, or a nucleobase analog.

In an embodiment thereof, R₆ is OC₁-C₃-alkyl, preferably wherein R₆ is OCH₃, wherein the dashed methylene bridge between R₆ and the 4′ C is absent.

In another embodiment thereof, R₈ is OC₁-C₃-alkyl, preferably wherein R₈ is OCH₃, wherein the dashed methylene bridge between R₈ and the 4′ C is absent.

In yet another embodiment thereof, R₆ is OC₁-C₃-alkyl, preferably wherein R₆ is OCH₃, wherein the dashed methylene bridge between R₆ and the 4′ C is absent; and R₈ is OC₁-C₃-alkyl, preferably wherein R₈ is OCH₃, wherein the dashed methylene bridge between R₈ and the 4′ C is absent.

In still another embodiment thereof, (i) n₃ is 1; (ii) each of X₂ through X₈ is O; (iii) each of Y₁ through Y₅ is O; (iv) each of Z₁ through Z₅ is OH; and (v) each of ring B₂ through ring B₄ is a nucleobase.

In the fourth aspect, the present invention relates to an RNA molecule comprising at least three nucleotides and comprising a 5′ terminal acyclonucleoside, wherein the acyclonucleoside comprises a linear unbranched structure or a linear single-branched structure instead of a ribose, and wherein the 5′ terminal acyclonucleoside is optionally deuterated.

In an embodiment of the fourth aspect, the linear unbranched structure has the structure of formula (II):

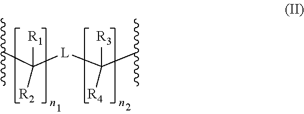

wherein
each of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).

In an embodiment thereof, each of R₁ through R₄ is independently H or OH. In another embodiment thereof, each of R₁ through R₃ is H and R₄ is H or OH. In another embodiment thereof, each of R₁ through R₄ is H.

In another embodiment of the fourth aspect, the linear single-branched structure has the structure of formula (II):

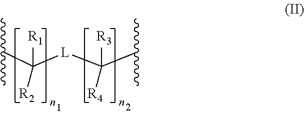

wherein
one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).

In an embodiment thereof, one of R₁ through R₄ is selected from the group consisting of CH₃, CH₂(OH), and CH(OH)₂, and each of the remaining three of R₁ through R₄ is independently H or OH.

In yet another embodiment relating to the above fourth aspect, n₁ and n₂ are each independently selected from an integer ranging from 0 to 3. It can be preferred that n₁ is selected from 0, 1, 2 or 3; and n₂ is selected from 0, 1 or 2. It can also be preferred that n₁ is 0; and n₂ is selected from 1 or 2. It can also be preferred that n₁ is 1; and n₂ is selected from 1 or 2. Still further, it can be preferred that n₁ is 2; and n₂ is selected from 1 or 2. Also, it can be preferred that n₁ is selected from 1 or 2; and n₂ is 0. It can be preferred that n₁ is selected from 1 or 2; and n₂ is 1. Yet in another preferred embodiment, n₁ is selected from 1 or 2; and n₂ is 2. It can still be preferred that n₁ is 3; and n₂ is 1 or that n₁ is 2; and n₂ is 0.

In another embodiment thereof, L is selected from the group consisting of CH₂, O, S, SO, SO₂ and CH(OH).

In yet another embodiment thereof, each of R₁ through R₄ is independently H or OH; n₁ and n₂ are each independently selected from an integer ranging from 0 to 3; and L is selected from the group consisting of CH₂, O, S, SO, SO₂ and CH(OH).

In another preferred embodiment thereof, (i) each of R₁ through R₄ is H; (ii) n₁ is selected from 0, 1 or 2; (iii) n₂ is selected from 1 or 2; and (iv) L is selected from CH₂ and O.

In yet another preferred embodiment thereof, (i) each of R₁ through R₃ is H; (ii) R₄ is H or OH; (iii) each of n₁ and n₂ is selected from 1 or 2; and (iv) L is selected from CH_{2, 0} and CH(OH).

In still another preferred embodiment thereof, (i) each of R₁ through R₄ is H; (ii) n₁ is selected from 1, 2 or 3; (iii) n₂ is selected from 0, 1 or 2; and (iii) L is selected from S, SO and SO₂.

In yet another embodiment thereof, one of R₁ through R₄ is selected from the group consisting of CH₃, CH₂(OH), and CH(OH)₂, and each of the remaining three of R₁ through R₄ is independently H or OH, preferably one of R₃ through R₄ is selected from the group consisting of CH₃, CH₂(OH), and CH(OH)₂, and each of the remaining three of R₁ through R₄ is independently H or OH; (ii) n₁ and n₂ are each independently selected from an integer ranging from 0 to 3; and (iii) L is selected from the group consisting of CH₂, O, S, SO, SO₂ and CH(OH).

In another preferred embodiment thereof, (i) one of R₁ through R₄ is selected from the group consisting of CH₃, CH₂(OH), and CH(OH)₂, and each of the remaining three of R₁ through R₄ is H, preferably one of R₃ through R₄ is selected from the group consisting of CH₃, CH₂(OH), and CH(OH)₂, and each of the remaining three of R₁ through R₄ is H; (ii) n₁ is selected from 0, 1 or 2; (iii) n₂ is selected from 1 or 2; and (iv) L is selected from CH₂ and O.

In yet another preferred embodiment thereof, (i) one of R₁ through R₄ is selected from the group consisting of CH₃, CH₂(OH), and CH(OH)₂, and each of the remaining three of R₁ through R₄ is H, preferably one of R₃ through R₄ is selected from the group consisting of CH₃, CH₂(OH), and CH(OH)₂, and each of the remaining three of R₁ through R₄ is H; (ii) each of n₁ and n₂ is selected from 1 or 2; and (iii) L is selected from CH₂, O and CH(OH).

In still another preferred embodiment thereof, (i) one of R₁ through R₄ is CH₃, and each of the remaining three of R₁ through R₄ is H, preferably one of R₃ through R₄ is CH₃, and each of the remaining three of R₁ through R₄ is H; (ii) n₁ is selected from 1, 2 or 3; (iii) n₂ is selected from 0, 1 or 2; and (iii) L is selected from S, SO and SO₂.

In a preferred embodiment of the fourth aspect, the acyclonucleoside comprises as the nucleobase guanine, a modified guanine or a guanine analog. It can be especially preferred that the acyclonucleoside comprises as the nucleobase a modified guanine, most preferably N⁷-methylguanine.

In the fifth aspect, the present invention relates to an RNA molecule whose 5′ end comprises a compound according to the first aspect.

It is noted with respect to the fifth aspect that the compound according to the first aspect mandatorily has an OH-group at the 3-position of the ribose of the 3′ nucleoside as shown in the following, namely due to (i) the definition of R₇ being OH or (ii) the alternative definition of R₇ if R₇ is not OH:

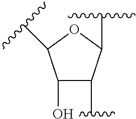 [SMILES: *C1OC(*)C(O)C1*]

It is at this OH-group at the 3-position of the ribose where the further nucleotides in 3′ direction of the RNA molecule form a covalent bond, as shown here:

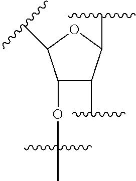 [SMILES: *OC1C(*)OC(*)C1*]

Thus, the RNA molecule of the fifth aspect comprises the compound according to the first aspect at its 5′ end such that this compound is covalently bound to the remainder of the RNA molecule, wherein the compound according to the first aspect is comprised in the cap structure of the RNA molecule.

Of course, all embodiments of the first aspect as outlined above also apply for the compounds that are comprised in the RNA molecule of the fifth aspect.

In the sixth aspect, the present invention is concerned with an in vitro method for synthesizing an RNA molecule, the method comprising reacting nucleotides, (i) the compound according to the first aspect or (ii) the cap analog according to the second aspect, and a DNA template in the presence of a DNA-dependent RNA polymerase under conditions suitable for the transcription of the DNA template into an RNA molecule by the DNA-dependent RNA polymerase.

The sixth aspect may alternatively be formulated as an in vitro method for synthesizing a capped RNA molecule, the method comprising reacting nucleotides, (i) a compound according to the first aspect or (ii) a cap analog according to the second aspect, and a DNA template in the presence of a DNA-dependent RNA polymerase under conditions suitable for the transcription of the DNA template into an RNA molecule by the DNA-dependent RNA polymerase.

In another preferred embodiment of the sixth aspect, the nucleotides are ATP, CTP, GTP and UTP. If the RNA is artificial RNA, modified nucleotides as set out below in the detailed description of the present invention may alternatively or additionally be used. Such nucleotides comprise at least one chemical modification that will also be present in the resulting RNA such that the resulting RNA is an artificial RNA according to the below definition.

The ratio of the compound according to the first aspect to the nucleotide GTP used in the method according to the sixth aspect may vary from 10:1 to 1:1 in order to balance the percentage of capped RNA products with the efficiency of the transcription reaction. Preferably, a ratio of the compound according to the first aspect to GTP of 4:1-6:1 is used.

In some embodiments of the sixth aspect, the method comprises at least one step of purifying the obtained capped RNA molecule. Suitable methods for purification may comprise RP-HPLC, Oligo-dT purification, cellulose-purification (such as e.g. the purification method using a cellulose material as disclosed in WO 2017/182525) and/or TFF.

In a preferred embodiment of the sixth aspect, the DNA-dependent RNA polymerase is the T7, T3 or SP6 polymerase.

In yet another preferred embodiment of the sixth aspect, the DNA template is a linearized DNA template with a promoter sequence that has a high binding affinity for its respective RNA polymerase.

In another preferred embodiment of the sixth aspect, the conditions suitable for the transcription of the DNA template into an RNA molecule comprise a suitable buffer, where the suitable buffer is preferably capable of maintaining a suitable pH value and may contain antioxidants (e.g. DTT), and/or polyamines such as spermidine at optimal concentrations. It can further be preferred that the buffer contains divalent cations, most preferably MgCl₂.

In yet another preferred embodiment of the sixth aspect, the method may further comprise adding a ribonuclease inhibitor. In another embodiment of the sixth aspect, the method may further comprises adding a pyrophosphatase.

Of course, all embodiments of the first aspect as outlined above also apply for the compounds of the first aspect that are used in the method of the sixth aspect. The same applies with respect to the embodiments of the second aspect as outlined above that apply for the cap analog of the second aspect that is used in the method of the sixth aspect.

In the seventh aspect, the present invention is concerned with an RNA molecule obtained by the method according to the sixth aspect, including all embodiments thereof.

In an embodiment of the seventh aspect, about 60%, about 65%, about 70%, about 75%, about 80%, about 85%, about 90%, or about 95% or more of the RNA molecules obtained by the method according to the sixth aspect comprises a cap structure derived from the compound according to the first aspect as determined using a capping assay. In preferred embodiments, less than about 30%, about 25%, about 20%, about 15%, about 10%, about 5%, about 4%, about 3%, about 2%, or about 1% of the RNA molecules obtained by the method according to the sixth aspect does not comprises a cap structure, determined using a capping assay. The capping assay may be carried out along the lines as shown herein in Example 3.

In another embodiment of the seventh aspect, the RNA molecule is characterized by an absence of reverse cap structures as compared to, e.g., RNA that has been generated using mCap. The structure of mCap (which may also be referred to as “mCap analog”) is shown in example 2 herein. The capping assay may essentially be carried out as shown herein in Example 3.

In another embodiment of the seventh aspect, the RNA molecule has a reduced dsRNA content as compared to, e.g., RNA that has been generated using mCap or RNA that has been generated by a post-transcriptional enzymatic capping reaction. The dsRNA content may be determined along the lines as shown herein in Example 4.

In embodiments relating to the third, fourth, fifth and seventh aspect, the RNA molecule comprises at least one chemical modification. The chemical modification may in particular be selected from the group consisting of a base modification, a sugar modification and a backbone modification. Such modifications are set out in detail in the detailed description of the present invention below. At least one chemical modification may in particular be a base modification, wherein the base modification is preferably selected from the group consisting of pseudouridine (psi or ψ), N1-methylpseudouracil (N1Mpsi or N1Mψ), 1-ethylpseudouracil, 2-thiouracil (s2U), 4-thiouracil, 5-methylcytosine, 5-methyluracil, 5-methoxyuracil, and any combination thereof. It can also be preferred that the base modification is selected from the group consisting of pseudouridine (ψ), N1-methylpseudouridine (m1ψ), 5-methylcytosine and 5-methoxyuridine.

In other embodiments relating to the third, fourth, fifth and seventh aspect, the RNA molecule does not comprise at least one chemical modification (i.e. no additional modification to the cap structure).

In particularly preferred embodiments relating to the third, fourth, fifth and seventh aspect, the RNA molecule is a coding RNA comprising at least one coding sequence. Most preferably, the coding RNA is an mRNA.

In preferred embodiments relating to the third, fourth, fifth and seventh aspect, the RNA molecule comprises at least one poly(A) sequence, and/or at least one poly(C) sequence, and/or at least one histone stem-loop and/or at least one 5′-UTR and/or at least one 3′-UTR.

In preferred embodiments relating to the third, fourth, fifth and seventh aspect, the RNA molecule is a therapeutic mRNA. As used herein, the term “therapeutic mRNA” refers to an RNA that encodes a therapeutic protein. Therapeutic proteins mediate a variety of effects in a host cell or a subject in order to treat a disease or ameliorate the signs and symptoms of a disease.

In preferred embodiments relating to the third, fourth, fifth and seventh aspect, the RNA molecule has an increased translation efficiency as compared to, e.g., natural RNA or RNA that has been generated using mCap.

In other preferred embodiments relating to the third, fourth, fifth and seventh aspect, the RNA molecule has an increased half-life as compared to, e.g., natural RNA or RNA that has been generated using mCap.

In still other preferred embodiments relating to the third, fourth, fifth and seventh aspect, the RNA molecule has an increased resistance to degradation as compared to, e.g., natural RNA or RNA that has been generated using mCap.

In still other preferred embodiments relating to the third, fourth, fifth and seventh aspect, the RNA molecule has an increased stability as compared to, e.g., RNA that has been generated using mCap or RNA that has been generated by a post-transcriptional enzymatic capping reaction.

In still other preferred embodiments relating to the third, fourth, fifth and seventh aspect, the RNA molecule exhibits reduced immunostimulation as compared to, e.g., RNA that has been generated using mCap or RNA that has been generated by a post-transcriptional enzymatic capping reaction.

In the eighth aspect, the present invention relates to a composition comprising the RNA molecule according to any of the third, fourth or fifth aspect, including all embodiments thereof as outlined above. The composition may also comprise a plurality of RNA molecules according to any of the third, fourth or fifth aspect, including all embodiments thereof as outlined above.

In an embodiment of the eight aspect, the RNA comprised in the composition is formulated in at least one cationic or polycationic compound, e.g. cationic or polycationic peptides, cationic or polycationic proteins, cationic or polycationic lipids, cationic or polycationic polysaccharides and/or cationic or polycationic polymers. In a preferred embodiment thereof, the RNA is formulated in lipid-based carriers, preferably wherein the lipid-based carriers encapsulate the RNA. In most preferred embodiments thereof, the lipid-based carriers are liposomes, lipid nanoparticles, lipoplexes, and/or nanoliposomes. Suitably, the lipid-based carriers of the composition comprise at least one aggregation-reducing lipid (e.g. a PEG-lipid), at least one cationic lipid, at least one neutral lipid, and/or at least one steroid or steroid analog.

In preferred embodiments, the composition is a pharmaceutical composition, in particular in the embodiments of the third, fourth or fifth aspect, where the RNA is therapeutic mRNA. The pharmaceutical composition comprises at least one pharmaceutically acceptable carrier.

In the ninth aspect, the present invention relates to a kit comprising (i) the compound according to the first aspect or (ii) the cap analog of the second aspect, and a DNA-dependent RNA polymerase, wherein it can be preferred that the DNA-dependent RNA polymerase is the T7, T3 or SP6 polymerase. This kit is suitable for producing a capped RNA. Of course, all embodiments of the first aspect as outlined above also apply for the compounds comprised in the kit of the ninth aspect, and all embodiments of the second aspect as outlined above also apply for the cap analog comprised in the kit of the ninth aspect.

In an embodiment of the ninth aspect, the kit further comprises nucleotides, preferably ATP, CTP, GTP and UTP.

If the RNA is artificial RNA, modified nucleotides as set out below in the detailed description of the present invention may alternatively or additionally be comprised in the kit. Such nucleotides comprise at least one chemical modification that will also be present in the resulting RNA such that the resulting RNA is an artificial RNA according to the below definition.

In yet another embodiment of the ninth aspect, the kit further comprises a ribonuclease inhibitor. In another embodiment of the ninth aspect, the kit further comprises a pyrophosphatase.

In still another embodiment of the ninth aspect, the kit further comprises a buffer. Preferably, this buffer is capable of maintaining a suitable pH value and may contain antioxidants (e.g. DTT), and/or polyamines such as spermidine at optimal concentrations. It can be preferred that the buffer contains divalent cations, most preferably MgCl₂.

In the tenth aspect, the present invention relates to the use of (i) the compound according to the first aspect or (ii) the cap analog of the second aspect in an in vitro transcription reaction for producing a capped RNA molecule.

The tenth aspect may alternatively be formulated as the use of (i) the compound according to the first aspect or (ii) the cap analog of the second aspect in an in vitro transcription reaction for co-transcriptionally producing capped RNA.

Of course, all embodiments of the first aspect as outlined above also apply for the compounds that are used according to the tenth aspect. Accordingly, all embodiments of the second aspect as outlined above also apply for the cap analogs that are used according to the tenth aspect.

In the eleventh aspect, the present invention relates to a method of synthesizing the compound according to the first aspect. Preferred methods of synthesizing the compound according to the first aspect can be found in example 1 herein below.

In the twelfth aspect, the present invention relates a method of increasing the translation of an in vitro transcribed RNA in a cell or subject, the method comprising at least the steps of (i) synthesizing an RNA molecule according to the method of the sixth aspect and (ii) applying the obtained RNA molecule to a cell or subject. The obtained RNA molecule may also be referred to as capped RNA.

In the thirteenth aspect, the present invention relates a method of increasing the half-life of an in vitro transcribed RNA in a cell or subject, the method comprising at least the steps of (i) synthesizing an RNA molecule according to the method of the sixth aspect and (ii) applying the obtained RNA molecule to a cell or subject. The obtained RNA molecule may also be referred to as capped RNA.

In the fourteenth aspect, the present invention relates a method of increasing resistance to degradation of an in vitro transcribed RNA in a cell or subject, the method comprising at least the steps of (i) synthesizing an RNA molecule according to the method of the sixth aspect and (ii) applying the obtained RNA molecule to a cell or subject. The obtained RNA molecule may also be referred to as capped RNA.

In the fifteenth aspect, the present invention relates a method of increasing stability of an in vitro transcribed RNA in a cell or subject, the method comprising at least the steps of (i) synthesizing an RNA molecule according to the method of the sixth aspect and (ii) applying the obtained RNA molecule to a cell or subject. The obtained RNA molecule may also be referred to as capped RNA.

In the sixteenth aspect, the present invention relates a method of reducing immunostimulation of an in vitro transcribed RNA in a cell or subject, the method comprising at least the steps of (i) synthesizing an RNA molecule according to the method of the sixth aspect and (ii) applying the obtained RNA molecule to a cell or subject. The obtained RNA molecule may also be referred to as capped RNA.

All embodiments of the sixth aspect of course also apply for the methods of the twelfth to the sixteenth aspect.

In the seventeenth aspect, the present invention relates to a transcription initiation complex comprising (i) the compound according to the first aspect or (ii) the cap analog according to the second aspect, and a DNA template. It can be preferred that the DNA template is a linearized DNA template.

Of course, all embodiments of the first aspect as outlined above also apply for the compounds that are comprised in the complex according to the seventeenth aspect. Accordingly, all embodiments of the second aspect as outlined above also apply for the cap analogs that are comprised in the complex according to the seventeenth aspect.

In the eighteenth aspect, the present invention is concerned with an in vitro method for synthesizing an RNA molecule, the method comprising
(A) reacting (i) nucleotides, (ii) a compound of formula (I)

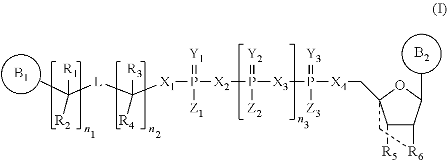

or a salt, stereoisomer, tautomer or deuterated version thereof, wherein
R₅ is OH;
ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
n₃ is selected from 0, 1 or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
each of X₁ through X₄ is independently O, S, NH or CH₂;
each of Y₁ through Y₃ is independently O, S or Se;
each of Z₁ through Z₃ is independently OH, SH or BH₃;
R₆ is OH, wherein the dashed methylene bridge between R₆ and the 4′ C is absent; and
ring B₂ is a nucleobase, a modified nucleobase or a nucleobase analog;
and (iii) a DNA template in the presence of a DNA-dependent RNA polymerase under conditions suitable for the transcription of the DNA template into an RNA molecule by the DNA-dependent RNA polymerase;
(B) reacting the resulting RNA molecule in the presence of an RNA-methyltransferase that catalyzes the methylation of the OH-group at R₅ to arrive at OCH₃ under conditions suitable for this methylation reaction; and
(C) thereby arriving at an RNA molecule.

The eighteenth aspect may alternatively be formulated as an in vitro method for synthesizing a capped RNA molecule with a cap1 structure, the method comprising
(A) reacting (i) nucleotides. (ii) a compound of formula (I)

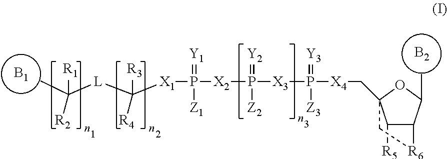

or a salt, stereoisomer, tautomer or deuterated version thereof, wherein
R₅ is OH;
ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
n₃ is selected from 0, 1 or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
each of X₁ through X₄ is independently O, S, NH or CH₂;
each of Y₁ through Y₃ is independently 0, S or Se;
each of Z₁ through Z₃ is independently OH, SH or BH₃;
R₆ is OH, wherein the dashed methylene bridge between R₆ and the 4′ C is absent; and
ring B₂ is a nucleobase, a modified nucleobase or a nucleobase analog;
and (iii) a DNA template in the presence of a DNA-dependent RNA polymerase under conditions suitable for the transcription of the DNA template into an RNA molecule by the DNA-dependent RNA polymerase;
(B) reacting the resulting RNA molecule in the presence of an RNA-methyltransferase that catalyzes the methylation of the OH-group at R₅ to arrive at OCH₃ under conditions suitable for this methylation reaction; and
(C) thereby arriving at a capped RNA molecule with a cap1 structure.

In another preferred embodiment of the eighteenth aspect, the nucleotides are ATP, CTP, GTP and UTP. If the RNA is artificial RNA, modified nucleotides as set out below in the detailed description of the present invention may alternatively or additionally be used. Such nucleotides comprise at least one chemical modification that will also be present in the resulting RNA such that the resulting RNA is an artificial RNA according to the below definition.

The ratio of the compound according to formula (I) to the nucleotide GTP used in the method according to the eighteenth aspect may vary from 10:1 to 1:1 in order to balance the percentage of capped RNA products with the efficiency of the transcription reaction. Preferably, a ratio of the compound according to the first aspect to GTP of 4:1-6:1 is used.

In some embodiments of the eighteenth aspect, the method comprises at least one step of purifying the obtained capped RNA molecule, optionally purifying the capped RNA molecule obtained after step (A) or purifying the capped RNA molecule with a cap1 structure obtained after step (B). Suitable methods for purification may comprise RP-HPLC, Oligo-dT purification, cellulose-purification (such as e.g. the purification method using a cellulose material as disclosed in WO 2017/182525) and/or TFF.

In a preferred embodiment of the eighteenth aspect, the DNA-dependent RNA polymerase is the T7, T3 or SP6 polymerase.

In yet another preferred embodiment of the eighteenth aspect, the DNA template is a linearized DNA template with a promoter sequence that has a high binding affinity for its respective RNA polymerase.

In another preferred embodiment of the eighteenth aspect, the conditions suitable for the transcription of the DNA template into an RNA molecule comprise a suitable buffer, where the suitable buffer is preferably capable of maintaining a suitable pH value and may contain antioxidants (e.g. DTT), and/or polyamines such as spermidine at optimal concentrations. It can further be preferred that the buffer contains divalent cations, most preferably MgCl₂.

In yet another preferred embodiment of the eighteenth aspect, the method may further comprise adding a ribonuclease inhibitor. In another embodiment of the sixth aspect, the method may further comprises adding a pyrophosphatase.

In a preferred embodiment of the eighteenth aspect, the RNA-methyltransferase that catalyzes the methylation of the OH-group at R₅ to arrive at OCH₃ is a 2′-O-Methyltransferase, for example a 2′-O-Methyltransferase derived from Vaccinia virus (e.g. ScriptCap from Cellscript).

In another preferred embodiment of the eighteenth aspect, the conditions suitable for the methylation of the OH-group at R₅ to arrive at OCH₃ comprise a suitable buffer, where the suitable buffer is preferably a 1× ScriptCap capping buffer from Cellscript with an optional addition of RNase inhibitor and 20 mM S-Adenosyl methionine.

In the nineteenth aspect, the present invention is concerned with a process for preparing a compound of formula (I):

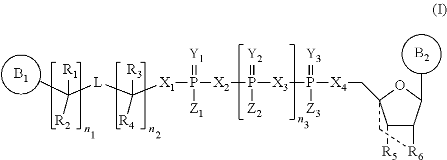

or a salt, stereoisomer, tautomer, or deuterated version thereof, wherein
R₅ is

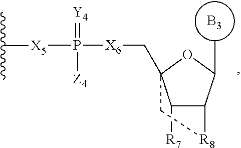

wherein
R₇ is OH or

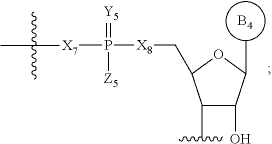

ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
n₃ is 0, 1, or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
each of X₁ through X₈ is independently O, S, NH or CH₂;
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) O, wherein the dashed methylene bridge between R₆ being O and the 4′ C is present;
R₈ is (i) selected from the group consisting of H, OH, OC₁-C₃-alkyl, and Opropargyl, wherein the dashed methylene bridge between R₈ and the 4′ C is absent, or (ii) O, wherein the dashed methylene bridge between R₈ being O and the 4′ C is present; and
each of ring B₂ through ring B₄ is independently a nucleobase, a modified nucleobase or a nucleobase analog;
   wherein the process comprises reacting
   a compound of formula (IV)

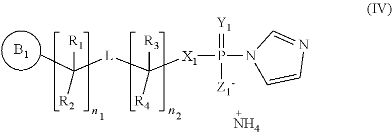

wherein B₁, R₁, R₂, R₃, R₄, n₁, n₂, X₁, Y₁, Z₁ are as defined above for formula (I); with
a compound of formula (V)

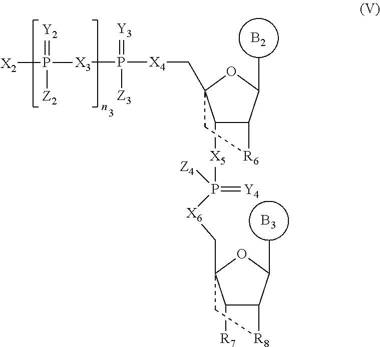

wherein X₂, Y₂, Z₂, X₃, Y₃, Z₃, X₄, Y₄, Z₄, X₅, X₆, R₆, R₇, R₈, B₂, and B₃ are as defined above for formula (I).

It has surprisingly been found that the activation of the dinucleotide or trinucleotide of formula (V) to form an activated dinucleotide or trinucleotide in order to react with an inactivated B1-linker moiety of formula (VI) as shown below led to undesired intramolecular side reactions of the activated dinucleotide or activated trinucleotide. However, it has been found that the compound of formula (I) can be prepared by activating the B1-linker moiety (formula VI) with imidazole and reacting the activated B1-linker moiety of formula (IV) with an inactivated dinucleotide or trinucleotide. This allows for easy condensation of the inactivated dinucleotide or trinucleotide with various different B1-linker moieties.

In a preferred embodiment of the nineteenth aspect, the reaction is performed in the presence of a metal chloride, preferably zinc chloride, manganese chloride or magnesium chloride, more preferably magnesium chloride. Preferably, the metal chloride is used in excess compared to the compound of formula (IV), wherein an excess refers to at least 5 equivalents, preferably at least 10 equivalents compared to the compound of formula (IV). Performing the reaction in the presence of magnesium chloride increases the yield of the product.

In another preferred embodiment of the nineteenth aspect, the reaction is performed in an aqueous solution and/or an organic solvent, preferably in a mixture of water and acetonitrile or in a mixture of water and N-methylmorpholine.

In another preferred embodiment of the nineteenth aspect, the compound of formula (IV) is reacted with the compound of formula (V) in equimolar amounts.

In another preferred embodiment of the nineteenth aspect, the product is desalted and purified by reverse-phase HPLC.

In another preferred embodiment of the nineteenth aspect, the process further comprises preparing the compound of formula (IV) comprising reacting a compound of formula (VI)

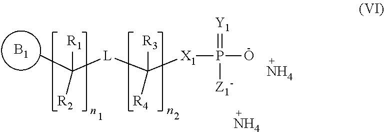

wherein B₁, R₁, R₂, R₃, R₄, n₁, n₂, X₁, Y₁, Z₁ are as defined above for formula (IV);
with carbonyldiimidazole.

It has surprisingly been found that using imidazole together with PPh3 and dipyridyldisulfide was not suitable for imidazole activation of the compound of formula (VI). However, using carbonyldiimidazole allows for imidazole activation of the compound of formula (VI) forming the compound of formula (IV). In particular, performing the reaction in DMSO provides high yields within 24-72 h, while performing the reaction in DMF is more slowly.

Thus, in another preferred embodiment of the nineteenth aspect, the reaction of a compound of formula (VI) with carbonyldiimidazole is performed in DMSO.

In another preferred embodiment of the nineteenth aspect, the compound of formula (VI) is reacted with an excess of carbonyldiimidazole. Using an excess of carbonyldiimidazole increases the yield of the compound of formula (IV). Preferably, the compound of formula (VI) is reacted with an excess of carbonyldiimidazole, wherein the excess refers to 2 to 40 equivalents, more preferably 10 to 25 equivalents, even more preferably 20 to 30 equivalents of carbonyldiimidazole relative to the compound of formula (VI). The excess of carbonyldiimidazole is preferably quenched after the reaction is finished.

In another preferred embodiment of the nineteenth aspect, excess carbonyldiimidazole is quenched with water. Surprisingly it has been found that quenching excess carbonyldiimidazole with water provides the desired product. In contrast, quenching excess carbonyldiimidazole with methanol does not lead to an observable product formation.

In a preferred embodiment of the nineteenth aspect, the preferred embodiments disclosed in connection with formula (I) of the first aspect also apply to formula (I), formula (IV), formula (V), and formula (VI) of the nineteenth aspect.

In one preferred embodiment,
n₃ is 1;
X₁ is O or CH₂;
each of X₂ through X₈ is O;
each of Y₁ through Y₅ is O;
each of Z₁ through Z₅ is OH;
R₆ is selected from the group consisting of H, OH, OC₁-C₃-alkyl, and Opropargyl, wherein the dashed methylene bridge between R₆ and the 4′ C is absent, preferably R₆ is OCH₃;
R₈ is selected from the group consisting of H, OH, OC₁-C₃-alkyl, and Opropargyl, wherein the dashed methylene bridge between R₈ and the 4′ C is absent, preferably R₈ is OCH₃.

In a more preferred embodiment,
n₃ is 1;
X₁ is O or CH₂;
each of X₂ through X₈ is O;
each of Y₁ through Y₅ is O;
each of Z₁ through Z₅ is OH;
R₆ is selected from the group consisting of H, OH, OC₁-C₃-alkyl, and Opropargyl, wherein the dashed methylene bridge between R₆ and the 4′ C is absent, preferably R₆ is OCH₃;
R₇ is OH.

It is understood that the process for preparing a compound of formula (I) of the nineteenth aspect is suitable for preparing all embodiments outlined above in the first aspect. Thus, the nineteenth aspect may also be formulated as a process for preparing a compound of formula (I) as defined above in the present aspect and/or all embodiments of formula (I) as described above in the first aspect.

### BRIEF DESCRIPTION OF THE DRAWINGS

FIG. 1 shows the structures of exemplary compounds obtained and obtainable by the synthesis route I described in examples 1.1 and 1.4.

FIG. 2 shows the structures of exemplary compounds obtained and obtainable by the synthesis route II described in example 1.2.

FIG. 3 shows the structures of exemplary compounds obtained and obtainable by the synthesis route III described in example 1.3.

FIG. 4 shows starting materials and the resulting cap analogs wherein the substitution pattern (and optionally the chain length of the carbon linker) is different from the pattern (and length) in compound 20.

FIG. 5 shows the PpLuc protein expression in HDF and HeLa cells 24 h after transfection of 50 ng cap0 mRNA constructs. Further details are provided in Example 5.

FIG. 6 shows PpLuc protein expression in HDF cells 24 h after transfection of 50 ng cap1 mRNA constructs. Further details are provided in Example 5.

### DETAILED DESCRIPTION OF THE INVENTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

### Definitions

For the sake of clarity and readability, the following scientific background information and definitions are provided. Any technical features mentioned herein or disclosed thereby can be part of or may be read on each and every embodiment of the invention. Additional definitions and explanations can be provided in the context of this disclosure.

As used in the specification and the claims, the singular forms of “a” and “an” also include the corresponding plurals unless the context clearly dictates otherwise. The same applies for plural forms used herein, which also include the singular forms unless the context clearly dictates otherwise.

The terms “about” and “approximately” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group, which preferably consists of these embodiments only.

The term “compound(s) of the present invention” is to be understood as equivalent to the terms “compound(s) according to the invention” and “compound(s) of the present application, and also covers a salt, stereoisomer, tautomer or N-oxide thereof.

The compounds according to the invention may be amorphous or may exist in one or more different crystalline states (polymorphs), which may have different macroscopic properties such as stability or show different biological properties such as activities. The present invention relates to amorphous and crystalline forms of compounds of formula (I), mixtures of different crystalline states of the compounds of formula (I), as well as amorphous or crystalline salts thereof.

The compounds according to the invention may be present in the form of salts. For example, the groups Z₁ through Z₅ if representing OH may typically be present in deprotonated form, i.e. as “O−” carrying a negative charge. At the same time, the nucleobases, modified nucleobases or a nucleobase analogs may, e.g., be present positively charged form. In particular, the group B₁ may, e.g., carry a positive charge, if B₁ represents N⁷-methylguanine. In addition, positively charged counterions may be present, such that pharmaceutically acceptable salts of the compounds according to the invention are formed.

Salts of the compounds according to the invention are preferably pharmaceutically acceptable salts, such as those containing counterions present in drug products listed in the US FDA Orange Book database. They can be formed in a customary manner, e.g., by reacting the compound with an acid of the anion in question, if the compounds according to the invention have a basic functionality, or by reacting acidic compounds according to the invention with a suitable base.

Suitable cationic counterions are in particular the ions of the alkali metals, preferably lithium, sodium and potassium, of the alkaline earth metals, preferably calcium, magnesium and barium, and of the transition metals, preferably manganese, copper, silver, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore the cations of 1,4-piperazine, meglumine, benzathine and lysine.

Suitable anionic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate, furthermore lactate, gluconate, and the anions of poly acids such as succinate, oxalate, maleate, fumarate, malate, tartrate and citrate, furthermore sulfonate anions such as besylate (benzenesulfonate), tosylate (p-toluenesulfonate), napsylate (naphthalene-2-sulfonate), mesylate (methanesulfonate), esylate (ethanesulfonate), and ethanedisulfonate. They can be formed by reacting compounds according to the invention that have a basic functionality with an acid of the corresponding anion.

Suitable counterions may also be introduced by applying ion exchange chromatography and/or using suitable buffers.

If the compounds according to the invention are present in the form of salts, the compounds themselves may contain positive and negative charges, and, in addition, counterions may be present for charge neutrality. For example, the groups Z₁ through Z₅, if representing OH, may typically be present in deprotonated form, i.e. as “O⁻” carrying a negative charge. At the same time, the nucleobases, modified nucleobases or a nucleobase analogs may, e.g., be present positively charged form. In particular, the group B₁ may, e.g., carry a positive charge, if B₁ represents N⁷-methylguanine, due to the attachment to the remainder of the molecule. In addition, positively charged counterions may be present, such that pharmaceutically acceptable salts of the compounds according to the invention are formed.

It is to be understood that also the precursors of the molecules may be present in charged as well as in non-charged form.

Depending on the substitution pattern, the compounds according to the invention may have one or more centers of chirality, including axial chirality. The invention provides both, pure enantiomers or pure diastereomers, of the compounds according to the invention, and their mixtures, including racemic mixtures. Suitable compounds according to the invention also include all possible geometrical stereoisomers (cis/trans isomers or E/Z isomers) and mixtures thereof. E/Z-isomers may be present with respect to, e.g., an alkene, carbon-nitrogen double-bond or amide group.

Tautomers may be formed, if a substituent is present at the compound of formula (I), which allows for the formation of tautomers such as keto-enol tautomers, imine-enamine tautomers, amide-imidic acid tautomers or the like.

The term “is deuterated” as used herein means that at least one of the hydrogen atoms occurring in the respective moiety is replaced by deuterium. Thus, if e.g. a nucleoside is deuterated, at least one of the hydrogen atoms occurring in the sugar and the nucleobase of the nucleoside is replaced by deuterium. The deuteration of a respective moiety may be partial in the sense that one or more but not all hydrogen atoms occurring in the respective moiety is/are replaced by deuterium. The afore-mentioned definition also applies to the term “a deuterated version thereof”, which refers to a compound of a given structure (such as e.g. the structure of formula (i) of the present application), and wherein at least one of the hydrogen atoms occurring in this given structure is replaced by deuterium.

The term “deuterium” as used herein has its common meaning, i.e. it is an isotope of hydrogen. In comparison with hydrogen, deuterium has an additional neutron. It is also referred to as “hydrogen-2” or “heavy hydrogen” and is abbreviated as “D” or “²H”.

A deuteration may have a positive impact, such as e.g. a reduced immunogenicity and/or enhanced expression of an RNA (see WO 2019/158583 for details) or e.g. an increased resistance to thermal and enzymatic hydrolysis (see WO 2022/099411 for details).

The term “substituted”, as used herein, means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The organic moieties mentioned in the above definitions of the variables are—like the term halogen—collective terms for individual listings of the individual group members. The prefix Cn-Cm indicates in each case the possible number of carbon atoms in the group.

The term “halogen” denotes in each case fluorine, bromine, chlorine or iodine, in particular fluorine, chlorine, or bromine.

The term “alkyl” as used herein denotes in each case a straight-chain or branched alkyl group having usually from 1 to 6 carbon atoms, preferably 1 to 5 or 1 to 4 carbon atoms, more preferably 1 to 3 or 1 or 2 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “nucleic acid” means any compound comprising, or preferably consisting of, DNA or RNA. The term may be used for a polynucleotide and/or oligonucleotide.

The term “DNA” is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers or analogs thereof which are—by themselves—composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single stranded or double stranded. In the double stranded form, the nucleotides of the first strand typically hybridize with the nucleotides of the second strand, e.g. by A/T-base-pairing and G/C-base-pairing.

The term “RNA” is the usual abbreviation for ribonucleic acid. It is A nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. RNA can be obtained by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional modifications such as splicing, 5-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, a coding sequence, optionally a 3′UTR and a poly(A) sequence. If RNA molecules are of synthetic origin, as in the present application, the RNA molecules are meant not to be produced in vivo, i.e. inside a cell or purified from a cell, but in an in vitro method. An examples for a suitable in vitro method is in vitro transcription. In addition to messenger RNA, several non-coding types of RNA exist which may be involved in regulation of transcription and/or translation, and immunostimulation and which may also be produced by in vitro transcription. The term “RNA” further encompasses RNA molecules, such as viral RNA, retroviral RNA and replicon RNA, small interfering RNA (siRNA), antisense RNA, saRNA (small activating RNA), CRISPR RNA (small guide RNA, sgRNA), ribozymes, aptamers, riboswitches, immunostimulating RNA, transfer RNA (tRNA), ribosomal RNA (rRNA), transfer-messenger RNA (tmRNA), small nuclear RNA (snRNA), small nucleolar RNA (snoRNA), microRNA (miRNA), and Piwi-interacting RNA (piRNA). A particularly preferred RNA molecule of the present invention is selected from the group consisting of mRNA, snRNA, snoRNA, tRNA, rRNA and tmRNA.

As noted above, in eukaryotic cells, a “cap structure” is typically found at the 5′ end of mRNAs, wherein this cap structure comprises of a “cap” nucleoside being 7-methylguanosine and a triphosphate bridge, wherein the triphosphate bridge forms a 5′ to 5 triphosphate linkage between the 5′ position of the ribose of the 7-methylguanosine and the 5′ position of the ribose of the first regular nucleotide of the mRNA. The cap structure facilitates translation or localization and/or prevents degradation of the mRNA when present at the 5′ end. If the ribose of the first and second nucleotide following the cap structure is not modified, this structure is referred to as “cap0 structure”. If the ribose of the first nucleotide following the cap structure carries an OCH₃ substituent at the 2′ position, the structure is referred to as “cap1 structure”. Finally, if the riboses of the first and second nucleotide following the cap structure carry an OCH₃ substituent at the 2′ position, the structure is referred to as “cap2 structure”. The cap structure (alternatively referred to as m⁷G5′ppp5′N) can be depicted as follows, wherein this structure may more particularly be referred to as cap2 structure:

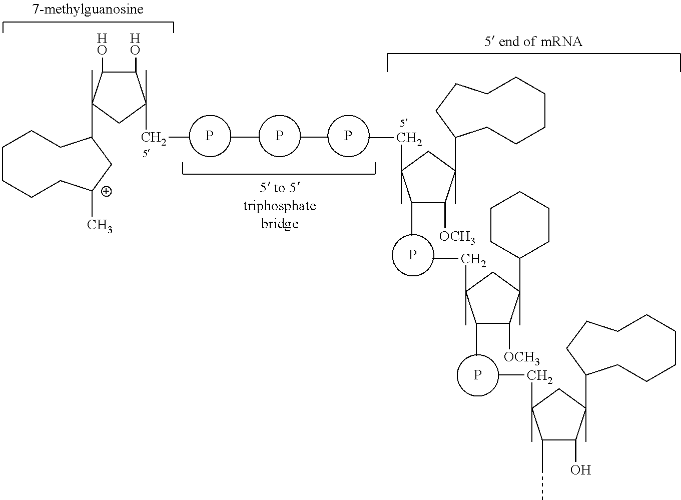

As outlined above, such cap structures can be achieved co-transcriptionally in in vitro transcription assays when using a “cap analog” in the in vitro transcription reaction. Accordingly, a “cap analog” may also be referred to as “cap analog for initiating RNA in vitro transcription”.

A variety of cap analogs has been developed and is commercially available for use in in vitro transcription reactions. Such cap analogs typically have structures corresponding to or mimicking a dinucleotide (also referred to as “cap0 analog”), a trinucleotide, where the ribose of the second nucleotide typically carries an OCH₃ substituent at the 2′ position (also referred to as “cap1 analog”) or a tetranucleotide, where the riboses of the second and third nucleotide typically carry a OCH₃ substituent at the 2′ position (also referred to as “cap2 analog”). All cap analogs have in common that they comprise 7-methylguanosine or an analog thereof at the position, where the 7-methylguanosine is found in a natural cap structure (which is at the 5F terminal position of the cap analog). Accordingly, if a 7-methylguanosine analog is used, this analog is used to mimic the natural 7-methylguanosine, and it is found at the position of the 7-methylguanosine. If an analog of 7-methylguanosine is used in a cap analog, the 7-methylguanosine analog comprises either (i) a ribose or (ii) a cyclic structure different from a ribose or (iii) a linear branched structure (mimicking the ribose) at the position, where a ribose is found in 7-methylguanosine. Examples of such cap analogs are shown in the following, wherein the ribose, cyclic structure or linear branched structure is encircled (the definitions of the specific substituents depicted in the following can be taken from the patent reference as indicated):
(i) The cap analog of WO 2009/149253, in particular the cap analog of claim 1 of WO 2009/149253 with the following structure:

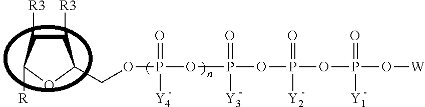

(ii) The cap analog of WO 2017/066781, in particular the cap analog of claim 1 of WO 2017/066781 with the following structure:

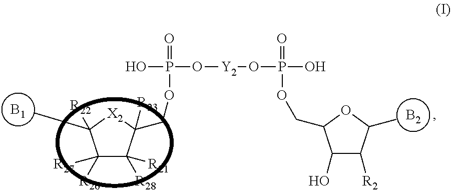

(iii) The cap analog of WO 2017/066782, in particular the cap analog of claim 1 of WO 2017/066782 with the following structure:

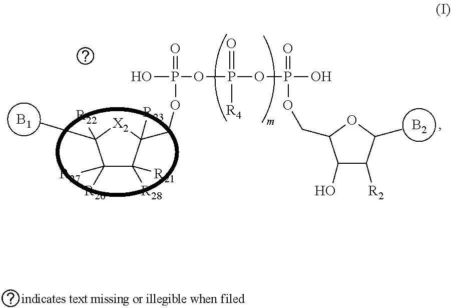

(iv) The cap analog of WO 2017/066789, in particular the cap analog of claim 1 of WO 2017/066789 with the following structure:

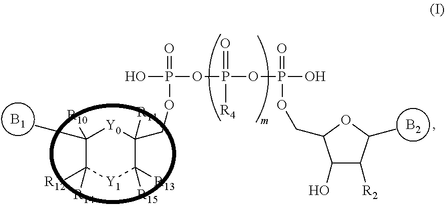

(v) The cap analog of WO 2017/053297, in particular the cap analog of claim 1 of WO 2017/053297 with the following structure:

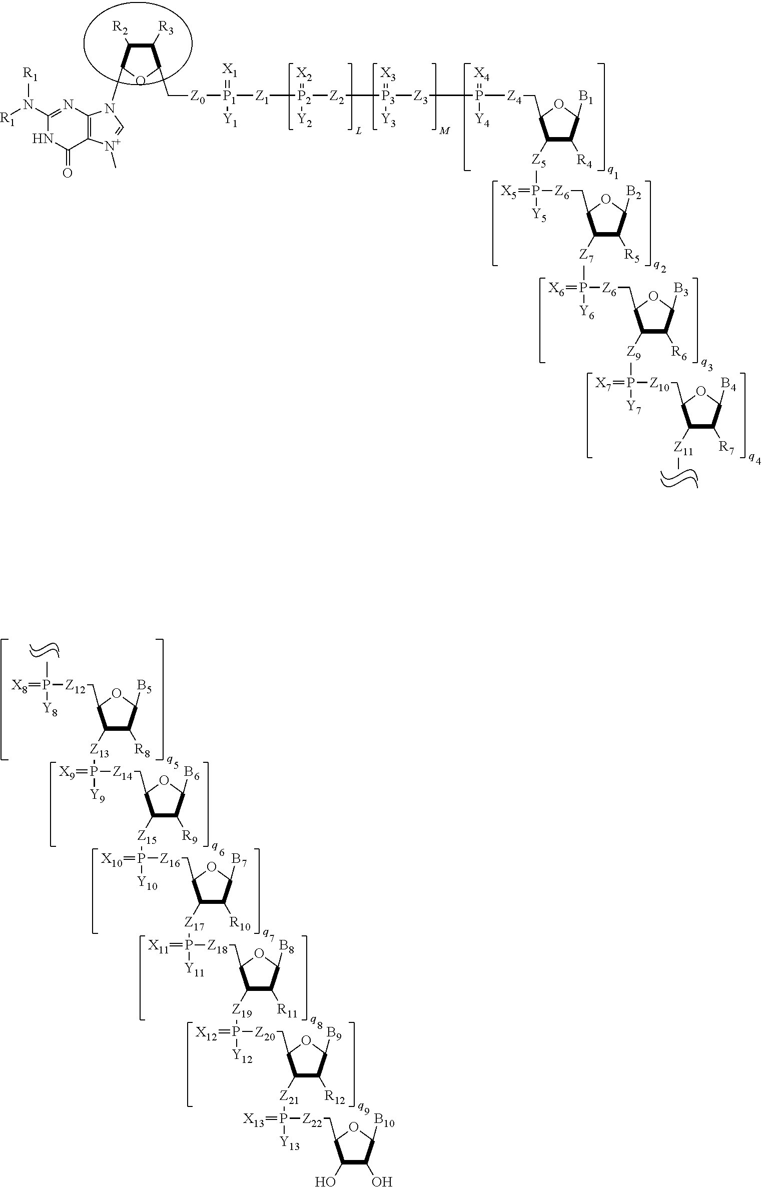

(vi) The cap analog of WO 2018/075827, in particular the cap analog of claim 1 of WO 2018/075827 with the following definition:

### Substitute Sheet (Rule 26)

A “cap analog comprising a 5′ terminal acyclonucleoside” may also be referred to as “cap analog comprising an acyclonucleoside at the position of the 7-methylguanosine” (in other words, at the position, where in a natural cap structure the 7-methylguanosine is found, or in still other words, at the position, where in cap analogs the 7-methylguanosine or an analog thereof is found) or as “cap analog comprising an acyclonucleoside at the position of the 7-methylguanosine and mimicking the 7-methylguanosine”. The cap analog comprising a 5′ terminal acyclonucleoside may be a cap0 analog, a cap1 analog or a cap2 analog, wherein a cap1 analog can be preferred, and the cap analog may be deuterated.

It has been found in the present invention that a ribose or another cyclic structure or a linear branched structure (mimicking the ribose), wherein linear branched structure is to be understood such that the branched structure is symmetric (i.e. as in the cap analog of claim 1 of WO 2017/066789, where two symmetric carbon units, one carbon unit with substituents R₁₂ and R₁₄ and the other carbon unit with substituents R₁₃ and R₁₅ are present if the dashed bonds and thus Y₁ are absent), at this position is not mandatory in order to provide a functional cap analog. Accordingly, the term “acyclonucleoside” as used in the present application refers to a structure that comprises a nucleobase, which is preferably guanine, a modified guanine or a guanine analog, and a linear unbranched structure or a linear single-branched structure at the position, where otherwise a ribose or another cyclic structure or a linear, (symmetric) branched structure (mimicking the ribose) is found. The term “unbranched” means in this respect that no carbon-containing substituents are present on the linear structural element, wherein the linear structural element is mainly made of carbon-units. The “linear unbranched structure” will be selected such that the resulting cap analog can still be used by the polymerase in the in vitro transcription reaction as transcription initiation compound. The term “single-branched” means in this respect that only a single carbon-containing substituent (or carbon unit) is present in the linear structural element (which may thus also be referred to as “asymmetric” with respect to the branch; contrary to the above symmetric branch with two carbon-containing substituents), wherein the linear structural element is mainly made of carbon-units. The “linear single-branched structure” will be selected such that the resulting cap analog can still be used by the polymerase in the in vitro transcription reaction as transcription initiation compound.

In view of the above, a “cap analog comprising a 5-terminal acyclonucleoside, wherein the acyclonucleoside comprises a linear unbranched structure instead of a ribose” may be any of the above-exemplified cap analogs, wherein the ribose or cyclic structure or linear branched structure of any of the above-exemplified cap analogs (with the ribose or cyclic structure or linear branched structure being encircled in the above-exemplified cap analogs) is substituted by a linear unbranched structure.

In view of the above, a “cap analog comprising a 5′-terminal acyclonucleoside, wherein the acyclonucleoside comprises a linear single-branched structure instead of a ribose” may be any of the above-exemplified cap analogs, wherein the ribose or cyclic structure or linear branched symmetric structure of any of the above-exemplified cap analogs (with the ribose or cyclic structure or linear symmetric branched structure being encircled in the above-exemplified cap analogs) is substituted by a linear single-branched structure.

The term “nucleoside” generally refers to compounds consisting of a sugar, usually ribose or deoxyribose, and a nucleobase, a modified nucleobase or a nucleobase analog as defined below. The nucleobase, modified nucleobase or nucleobase analog is attached to the carbon atom at the 1′ position of the ribose, as in naturally occurring nucleosides and as well-known to the skilled person. The nucleoside may be deuterated.

The term “nucleotide” generally refers to a nucleoside comprising at least one phosphate group, preferably one, two or three phosphate groups, attached to the sugar, in the ribose at the 5′ position.

The term “nucleobase” or simply “base” as used herein refers to the naturally occurring purines and pyrimidines that are present in DNA and RNA, in particular to adenine (A), cytosine (C), guanine (G), thymine (T) and uracil (U). The nucleobases A, G, C and T are found in DNA, whereas A, G, C and U are found in RNA. Accordingly, the nucleobases A, G, C and U are particularly relevant for the present invention. The structures of naturally occurring purines and pyrimidines that are present in DNA and RNA, in particular the structures of A, C, G, T and U, are well known to the skilled person and referred to herein. The nucleobase may be deuterated.

The term “modified nucleobase” as used herein refers to nucleobases as defined above, in particular A, C, G, T and U (with A, G, C and U being preferred for the present invention), which are modified in that the nucleobase carries an additional substituent, such as e.g. an amino group, a thiol group, an alkyl group (in particular a methyl group), or a halo group. Modified nucleobases may or may not be found in nature. For example, the nucleobases can be chemically modified on the major groove face. In some embodiments, the major groove chemical modifications can include an amino group, a thiol group, an alkyl group, or a halo group. Included are e.g. the modified nucleobases N⁶-methyladenine, hypoxanthine, xanthine, 7-methylguanine, 5,6-dihydrouracil, 5-methylcytosine and 5-hydroxymethylcytosine. The modified nucleobase may be deuterated.

The term “modified guanine” is thus a guanine that carried an additional substituent, such as e.g. a methyl-group. The additional substituent may, however, also be an amino group, a thiol group, an alkyl group different from methyl, or a halo group. A particularly preferred modified guanine is 7-methylguanine. The modified guanine may be deuterated.

The term “nucleobase analog” as used herein refers to an artificial, i.e. non-natural, nucleobase. A “nucleobase analog” is based on a nucleobase or a modified nucleobase as defined above, wherein not only an additional substituent may (in the case of a modified nucleobase) or may not (in the case of a nucleobase, in particular A, C, G, T or U) be present but at least one substitution can be found in the underlying purine and pyrimidine, respectively, of the nucleobase or modified nucleobase (e.g. a nitrogen in the purine or pyrimidine is substituted by a carbon). A nucleobase analog present in a nucleoside or a nucleotide can nevertheless substitute for a completely natural nucleoside or nucleotide, such as in particular for the nucleotides ATP, UTP, CTP and GTP. The nucleobase analog may be deuterated.

The term “guanine analog” is thus a guanine or a modified guanine, where an atom of the underlying purine structure has been substituted. An example of a guanine analog is 9-deazaguanine, and a particularly preferred guanine analog is 7-methyl-9-deazaguanine. Other examples for guanine analogs are 7-deaza-guanine, 7-cyano-7-deaza-guanine and 7-aminomethyl-7-deaza-guanine. The guanine analog may be deuterated.

In embodiments of the present invention, the modified nucleobase or the nucleobase analog is a nucleobase that is present in a nucleotide selected from the group consisting of 2-amino-6-chloropurineriboside-5′-tri-phosphate, 2-Aminopurine-riboside-5′-triphosphate; 2-aminoadenosine-5′-triphosphate, 2′-Amino-2′-deoxy-cytidine-triphosphate, 2-thiocytidine-5′-triphosphate, 2-thiouridine-5′-triphosphate, 2′-Fluorothymidine-5′-tri-phosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methyl-cytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurine-riboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. As an example, the nucleobase that is present in 5-methylcytidine-5′-triphosphate is 5-methylcytosine.

The modified nucleobase or the nucleobase analog is in particular a nucleobase that is present in a nucleotide selected from the group consisting of 5-methylcytidine-5′-triphosphate, 7-deazaguanosine-5′-triphosphate, 5-bromocytidine-5′-triphosphate, and pseudouridine-5′-triphosphate. As an example, the nucleobase that is present in 7-deazaguanosine-5′-triphosphate is 7-deazaguanine.

In some embodiments, the modified nucleobase or the nucleobase analog is a nucleobase that is present in a nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, 1-methoxymethyl-pseudouridine, 1-ethyl-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. As an example, the nucleobase that is present in 5-propynyl-uridine is 5-propynyl-uracil.

The modified nucleobase or the nucleobase analog is in some embodiments a nucleobase that is present in a nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. As an example, the nucleobase that is present in 2-thio-5-methyl-cytidine is 2-thio-5-methyl-cytosine.

In other embodiments, the modified nucleobase or the nucleobase analog is present in a nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. As an example, the nucleobase that is present in N6-glycinylcarbamoyladenosine is N6-glycinylcarbamoyladenine.

In other embodiments, the modified nucleobase or the nucleobase analog is a nucleobase that is present in a nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. As an example, the nucleobase that is present in 6-thio-7-methyl-guanosine is 6-thio-7-methyl-guanine.

In other embodiments of the present invention, the modified nucleobase or the nucleobase analog is a nucleobase that is present in a nucleoside selected from the group consisting of 6-aza-cytidine, 2-thio-cytidine, α-thio-cytidine, Pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine. As an example, the nucleobase that is present in 7-deaza-guanosine is 7-deaza-guanine.

In yet other embodiments of the present invention, the modified nucleobase or the nucleobase analog is a nucleobase that is present in a nucleoside selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. As an example, the nucleobase that is present in 2-thio-5-aza-uridine is 2-thio-5-aza-uracil.

In a specific embodiment, the modified nucleobase or the nucleobase analog is a nucleobase that is present in a nucleoside or nucleotide selected from the group consisting of pseudouracil (ψ), N1-methylpseudouracil (N1Mψ), 1-ethylpseudouracil, 2-thiouracil (s2U), 4-thiouracil, 5-methylcytosine, 5-methyluracil, 5-methoxyuracil, and any combination thereof, most preferably the chemical modification is N1-methylpseudouracil (N1Mψ). As an example, the nucleobase that is present in N1-methylpseudouracil is N1-methyluridine.

The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which is preferably a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention the DNA template is linearized with a suitable restriction enzyme, before it is transcribed in vitro. The cDNA may be obtained by reverse transcription of RNA or chemical synthesis. Moreover, the DNA template for in vitro RNA synthesis may also be obtained by gene synthesis.

Methods for in vitro transcription are known in the art (see, e.g., Geall et al. (2013) Semin. Immunol. 25(2): 152-159; Brunelle et al. (2013) Methods Enzymol. 530:101-14). Reagents used in said method typically include:
1) a linearized DNA template with a promoter sequence that has a high binding affinity for its respective RNA polymerase such as bacteriophage-encoded RNA polymerases;
2) ribonucleotides with triphosphates (NTPs), in particular ATP, CTP, GTP and UTP;
3) a DNA-dependent RNA polymerase capable of binding to the promoter sequence within the linearized DNA template (e.g. T7, T3 or SP6 RNA polymerase);
4) optionally, a ribonuclease (RNase) inhibitor to inactivate any contaminating RNase;
5) optionally, a pyrophosphatase to degrade pyrophosphate, which may inhibit transcription; 6) MgCl₂, which supplies Mg²⁺ ions as a co-factor for the polymerase;
7) a buffer to maintain a suitable pH value, which can also contain antioxidants (e.g. DTT), and/or polyamines such as spermidine at optimal concentrations; and
8) a cap analog (such as in particular a cap analog of the present invention).

Besides the acyclonucleoside at the position mimicking the 7-methylguanosine, the RNA according to the present application may comprise “artificial RNA”, wherein “artificial RNA” encompasses in particular RNA comprising at least one chemical modification. The chemical modification may be selected from the group consisting of a sugar modification, a backbone modification, and a base modification. A backbone modification in connection with the present invention is a modification in which phosphates of the backbone of the nucleotides contained in an RNA are chemically modified. A sugar modification in connection with the present invention is a chemical modification of the sugar of the nucleotides of the RNA. Furthermore, a base modification in connection with the present invention is a chemical modification of the nucleobase of the nucleotides of the RNA.

The modified nucleotides, which may be incorporated into RNA according to the present application, can be modified in the sugar. Accordingly, at least one sugar of the RNA of the present application may be modified. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethylene glycols (PEG), —O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., N^{RR}, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar can also contain one or more carbons that possess the opposite stereochemical configuration than that of the corresponding carbon in ribose. Thus, a modified RNA can include nucleotides containing, for instance, arabinose as the sugar.

The modified nucleotides, which may be incorporated into RNA according to the present application, can be modified in a phosphate group. Accordingly, at least a region of the backbone of the RNA of the present application may be modified. The phosphate groups of the backbone of the RNA according to the present application can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates). The backbone can also be modified in that it comprises or consists of repeating N-(2-aminoethyl)-glycine units linked by peptide bonds (so-called “peptide nucleic acid” or “PNA” backbones), where the nucleobases are linked to the backbone by a methylene bridge and a carbonyl group.

The modified nucleotides, which may be incorporated into RNA according to the present application in the in vitro reaction, can be modified in the nucleobase. Accordingly, at least one nucleobase of the RNA of the present application may be modified. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the modified nucleotides that are used in the in vitro transcription (thus resulting in the corresponding modification in the nucleobase in the resulting RNA) are selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5-triphosphate, 5-aminoallylcytidine-5′-triphosphate, 5-aminoallyluridine-5-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to modified nucleotides selected from the group consisting of 5-methylcytidine-5′-triphosphate, 7-deazaguanosine-5′-triphosphate, 5-bromocytidine-5′-triphosphate, and pseudouridine-5′-triphosphate. In some embodiments, the nucleotide can be modified on the major groove face and can include replacing hydrogen on C-5 of uracil with a methyl group or a halo group.

In some embodiments, the modified nucleotides that are used in the in vitro transcription (thus resulting in the corresponding modification in the nucleobase in the resulting RNA) are nucleotides that comprise modified nucleosides that include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine. In further specific embodiments, the modified nucleoside is selected from 6-aza-cytidine, 2-thio-cytidine, α-thio-cytidine, Pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine. In further embodiments, the modified nucleoside is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In a specific embodiment, the modified nucleoside is selected from the group consisting of pseudouracil (ψ), N1-methylpseudouracil (N1Mψ), 1-ethylpseudouracil, 2-thiouracil (s2U), 4-thiouracil, 5-methylcytosine, 5-methyluracil, 5-methoxyuracil, and any combination thereof, most preferably the modified nucleoside is N1-methylpseudouracil (N1Mψ).

A set of embodiments (A) of the present application relates to:
1. A compound of formula (I):

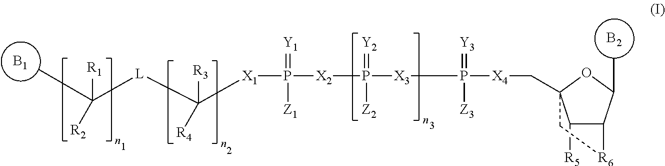

or a salt, stereoisomer or tautomer thereof, wherein
R₅ is OH or

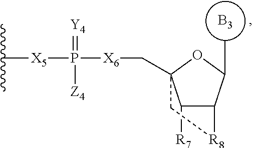

wherein
R₇ is OH or

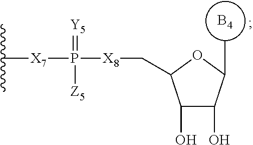

ring B₁ is guanine, a modified guanine or a guanine analog;
each of R₁ through R₄ is independently H, OH, SH, NH₂ or halogen;
n₁ and n₂ are each independently selected from an integer ranging from 0 to 10;
n₃ is selected from 0, 1 or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
each of X₁ through X₈ is independently O, S, NH or CH₂;
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) O, wherein the dashed methylene bridge between R₆ being 0 and the 4′ C is present;
R₈ is (i) selected from the group consisting of H, OH, OC₁-C₃-alkyl, and Opropargyl, wherein the dashed methylene bridge between R₈ and the 4′ C is absent, or (ii) O, wherein the dashed methylene bridge between R₈ being O and the 4′ C is present; and
each of ring B₂ through ring B₄ is independently a nucleobase, a modified nucleobase or a nucleobase analog.
2. The compound according to embodiment 1, wherein
   n₃ is 1;
   R₆ is selected from the group consisting of H, OH, OC₁-C₃-alkyl, and Opropargyl, wherein the dashed methylene bridge between R₆ and the 4′ C is absent; and
   R₈ is selected from the group consisting of H, OH, OC₁-C₃-alkyl, and Opropargyl, wherein the dashed methylene bridge between R₈ and the 4′ C is absent.
3. The compound according to embodiment 1 or 2, wherein B₂ is selected from the group consisting of guanine, a modified guanine, a guanine analog, adenine, a modified adenine, and an adenine analog.
4. The compound according to any one of the preceding embodiments, wherein B₃ is guanine, a modified guanine or a guanine analog.
5. The compound according to any one of the preceding embodiments, wherein R₅ is OH and wherein R₆ is OH, wherein the dashed methylene bridge between R₆ and the 4′ C is absent.
6. The compound according to any one of embodiments 1 to 4, wherein R₅ is

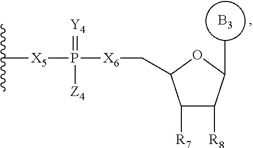

wherein R₇ and R₈ are each OH.
7. The compound according to embodiment 6, wherein R₆ is H or OC₁-C₃-alkyl, preferably wherein R₆ is OCH₃.
8. The compound according to any one of embodiments 1 to 4, wherein R₅ is

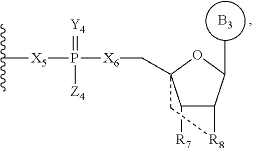

wherein R₇ is

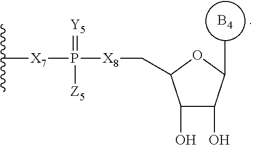

9. The compound according to embodiment 8, wherein
   R₆ is H or OC₁-C₃-alkyl, wherein the dashed methylene bridge between R₆ and the 4′ C is absent, preferably wherein R₆ is OCH₃; and/or
   R₈ is H or OC₁-C₃-alkyl, wherein the dashed methylene bridge between R₈ and the 4′ C is absent, preferably wherein R₈ is OCH_{3.}
10. The compound according to any one of the preceding embodiments, wherein ring B₁ is a modified guanine.
11. The compound according to any one of the preceding embodiments, wherein ring B₁ is N⁷-methylguanine.
12. The compound according to any one of the preceding embodiments, wherein each of X₂ through X₈ is 0.
13. The compound according to any one of the preceding embodiments, wherein each of Y₁ through Y₅ is 0.
14. The compound according to any one of the preceding embodiments, wherein each of Z₁ through Z₅ is OH.
15. The compound according to any one of the preceding embodiments, wherein each of R₁ through R₄ is independently H or OH.
16. The compound according to any one of the preceding embodiments, wherein each of R₁ through R₃ is H and R₄ is H or OH.
17. The compound according to any one of the preceding embodiments, wherein each of R₁ through R₄ is H.
18. The compound according to any one of the preceding embodiments, wherein n₁ and n₂ are each independently selected from an integer ranging from 0 to 3.
19. The compound according to any one of the preceding embodiments, wherein n₁ is selected from 0, 1, 2 or 3; and n₂ is selected from 0, 1 or 2.
20. The compound according to embodiment 19, wherein n₁ is 0; and n₂ is selected from 1 or 2.
21. The compound according to embodiment 19, wherein n₁ is 1; and n₂ is selected from 1 or 2.
22. The compound according to embodiment 19, wherein n₁ is 2; and n₂ is selected from 1 or 2.
23. The compound according to embodiment 19, wherein n₁ is selected from 1 or 2; and n₂ is 0.
24. The compound according to embodiment 19, wherein n₁ is selected from 1 or 2; and n₂ is 1.
25. The compound according to embodiment 19, wherein n₁ is selected from 1 or 2; and n₂ is 2.
26. The compound according to embodiment 19, wherein n₁ is 3; and n₂ is 1.
27. The compound according to embodiment 19, wherein n₁ is 2; and n₂ is 0.
28. The compound according to any one of the preceding embodiments, wherein L is selected from the group consisting of CH₂, O, S, SO, SO₂ and CH(OH).
29. The compound according to any one of embodiments 1 to 16, wherein (i) each of R₁ through R₄ is H; (ii) n₁ is selected from 0, 1 or 2; (iii) n₂ is selected from 1 or 2; (iv) L is selected from CH₂ and O; and (v) X₁ is O.
30. The compound according to any one of embodiments 1 to 16, wherein (i) each of R₁ through R₃ is H; (ii) R₄ is H or OH; (iii) wherein each of n₁ and n₂ is selected from 1 or 2; (iv) L is selected from CH₂, O and CH(OH); and (v) X₁ is O.
31. The compound according to any one of embodiments 1 to 16, wherein (i) each of R₁ through R₄ is H; (ii) n₁ is selected from 1, 2 or 3; (iii) n₂ is selected from 0, 1 or 2; (iii) L is selected from S, SO and SO₂; and
(iv) X₁ is CH₂.
32. A cap analog comprising a 5′ terminal acyclonucleoside, wherein the acyclonucleoside comprises a linear unbranched structure instead of a ribose.
33. The cap analog according to embodiment 32, wherein the linear unbranched structure has the structure of formula (II):

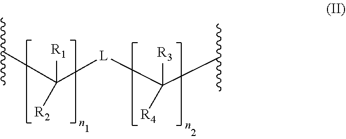

wherein
each of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).
34. The cap analog according to embodiment 33, wherein each of R₁ through R₄ is independently H or OH; n₁ and n₂ are each independently selected from an integer ranging from 0 to 3; and L is selected from the group consisting of CH₂, O, S, SO, SO₂ and CH(OH).
35. The cap analog according to any one of embodiments 32 to 34, wherein the acyclonucleoside comprises as the nucleobase guanine, a modified guanine or a guanine analog.
36. An RNA molecule comprising a 5′ end of formula (III):

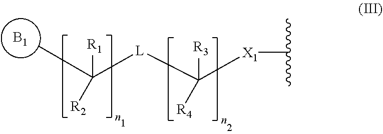

wherein
ring B₁ is guanine, a modified guanine or a guanine analog;
each of R₁ through R₄ is independently H, OH, SH, NH₂ or halogen;
n₁ and n₂ are each independently selected from an integer ranging from 0 to 10;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group
consisting of CH₂, CH(OH), CH(SH) and CH(halogen); and
X₁ is O, S, NH or CH_{2.}
37. The RNA molecule according to embodiment 36, wherein each of R₁ through R₄ is independently H or OH; n₁ and n₂ are each independently selected from an integer ranging from 0 to 3; L is selected from the group consisting of CH₂, O, S, SO, SO₂ and CH(OH); and X₁ is O or CH_{2.}
38. The RNA molecule according to embodiment 36 or 37, wherein ring B₁ is a modified guanine, preferably N⁷-methylguanine.
39. An RNA molecule comprising a 5′ terminal acyclonucleoside, wherein the acyclonucleoside comprises a linear unbranched structure instead of a ribose.
40. The RNA molecule according to embodiment 39, wherein the linear unbranched structure has the structure of formula (II):

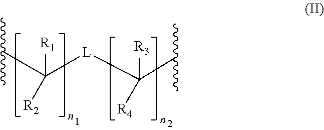

wherein
each of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).
41. The RNA molecule according to embodiment 40, wherein each of R₁ through R₄ is independently H or OH; n₁ and n₂ are each independently selected from an integer ranging from 0 to 3; and L is selected from the group consisting of CH₂, O, S, SO, SO₂ and CH(OH).
42. The RNA molecule according to any one of embodiments 39 to 41, wherein the acyclonucleoside comprises as the nucleobase guanine, a modified guanine or a guanine analog.
43. An RNA molecule whose 5′ end comprises a compound according to any one of embodiments 1 to 31.
44. An in vitro method for synthesizing an RNA molecule, the method comprising reacting nucleotides, (i) the compound according to any one of embodiments 1 to 31 or (ii) the cap analog according to any one of embodiments 32 to 35, and a DNA template in the presence of a DNA-dependent RNA polymerase under conditions suitable for the transcription of the DNA template into an RNA molecule by the DNA-dependent RNA polymerase.
45. An RNA molecule obtained by the in vitro method according to embodiment 44.
46. The RNA molecule according to any one of embodiments 36 to 43 and 45, wherein the RNA molecule comprises at least one chemical modification.
47. The RNA molecule according to embodiment 46, wherein the at least one chemical modification is selected from the group consisting of a base modification, a sugar modification and a backbone modification.
48. The RNA molecule according to embodiment 46 or 47, wherein the at least one chemical modification is a base modification, wherein the base modification is preferably selected from the group consisting of pseudouridine (psi or ψ), N1-methylpseudouracil (N1Mpsi or N1Mψ), 1-ethylpseudouracil, 2-thiouracil (s2U), 4-thiouracil, 5-methylcytosine, 5-methyluracil, 5-methoxyuracil, and any combination thereof.
49. The RNA molecule according to any one of embodiments 36 to 43 and 45 to 48, wherein the RNA molecule is a coding RNA comprising at least one coding sequence, preferably wherein the coding RNA is an mRNA.
50. The RNA molecule according to any one of embodiments 36 to 43 and 45 to 49, wherein the RNA molecule is a therapeutic mRNA.
51. A composition comprising the RNA molecule according to any one of embodiments 36 to 43 and 45 to 50.
52. The composition according to embodiment 51, wherein the composition is a pharmaceutical composition.
53. A kit comprising (i) the compound according to any one of embodiments 10 to 31 or (ii) the cap analog according to any one of embodiments 32 to 35, and a DNA-dependent RNA polymerase.
54. The kit according to embodiment 53, wherein the kit further comprises nucleotides.
55. The kit according to embodiment 53 or 54, wherein the kit further comprises a ribonuclease inhibitor.
56. The kit according to any one of embodiments 53 to 55, wherein the kit further comprises a buffer.
57. Use of (i) the compound according to any one of embodiments 1 to 31 or (ii) the cap analog according to any one of embodiments 32 to 35 in an in vitro transcription reaction for producing a capped RNA molecule.
58. The use according to embodiment 57, wherein the capped RNA molecule is the RNA molecule according to any one of embodiments 36 to 43 and 45 to 49.

A set of embodiments (B) of the present application relates to:
1. A compound of formula (I):

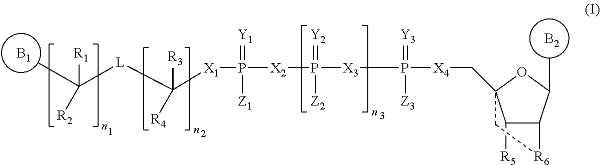

or a salt, stereoisomer, tautomer or deuterated version thereof, wherein
R₅ is OH or

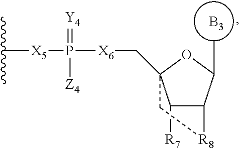

wherein
R₇ is OH or

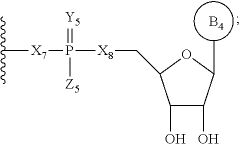

ring B₁ is a modified guanine or a modified guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
n₃ is selected from 0, 1 or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
each of X₁ through X₈ is independently O, S, NH or CH₂;
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) 0, wherein the dashed methylene bridge between R₆ being 0 and the 4′ C is present;
R₈ is (i) selected from the group consisting of H, OH, OC₁-C₃-alkyl, and Opropargyl, wherein the dashed methylene bridge between R₈ and the 4′ C is absent, or (ii) 0, wherein the dashed methylene bridge between R₈ being 0 and the 4′ C is present; and
each of ring B₂ through ring B₄ is independently a nucleobase, a modified nucleobase, or a nucleobase analog;
wherein the compound is not

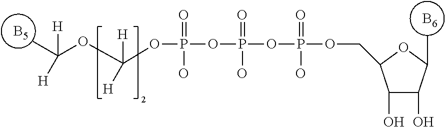

wherein B₅ is N⁷-methylguanine and B₆ is guanine.
2. The compound according to embodiment 1, wherein
   n₃ is 1;
   R₆ is selected from the group consisting of H, OH, OC₁-C₃-alkyl, and Opropargyl, wherein the dashed methylene bridge between R₆ and the 4′ C is absent; and
   R₈ is selected from the group consisting of H, OH, OC₁-C₃-alkyl, and Opropargyl, wherein the dashed methylene bridge between R₈ and the 4′ C is absent.
3. The compound according to embodiment 1 or 2, wherein B₂ is selected from the group consisting of guanine, a modified guanine, a guanine analog, an adenine, a modified adenine, and an adenine analog.
4. The compound according to any one of the preceding embodiments, wherein B₃ is guanine, a modified guanine, or a guanine analog.
5. The compound according to any one of the preceding embodiments, wherein R₅ is OH and wherein R₆ is OH, wherein the dashed methylene bridge between R₆ and the 4′ C is absent.
6. The compound according to any one of embodiments 1 to 4, wherein R₅ is

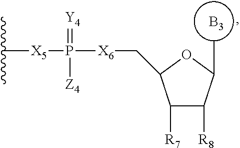

wherein R₇ and R₆ are each OH.
7. The compound according to embodiment 6, wherein R₆ is H or OC₁-C₃-alkyl, preferably wherein R₆ is OCH₃.
8. The compound according to any one of embodiments 1 to 4, wherein R₅ is

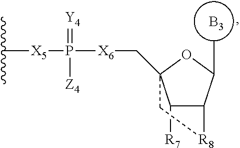

wherein R₇ is

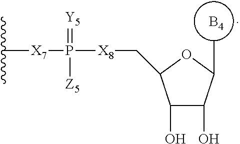

9. The compound according to embodiment 8, wherein
   R₆ is H or OC₁-C₃-alkyl, wherein the dashed methylene bridge between R₆ and the 4′ C is absent, preferably wherein R₆ is OCH₃; and/or
   R₈ is H or OC₁-C₃-alkyl, wherein the dashed methylene bridge between R₈ and the 4′ C is absent, preferably wherein R₈ is OCH_{3.}
10. The compound according to any one of the preceding embodiments, wherein ring B₁ is a modified guanine.
11. The compound according to any one of the preceding embodiments, wherein ring B₁ is N⁷-methylguanine.
12. The compound according to any one of the preceding embodiments, wherein each of X₂ through X₈ is 0.
13. The compound according to any one of the preceding embodiments, wherein each of Y₁ through Y₅ is 0.
14. The compound according to any one of the preceding embodiments, wherein each of Z₁ through Z₅ is OH.
15. The compound according to any one of the preceding embodiments, wherein each of R₁ through R₄ is independently H or OH; or one of R₁ through R₄ is selected from the group consisting of CH₃, CH₂(OH), and CH(OH)₂, and each of the remaining three of R₁ through R₄ is independently H or OH.
16. The compound according to any one of the preceding embodiments, wherein each of R₁ through R₃ is H and R₄ is H or OH.
17. The compound according to any one of the preceding embodiments, wherein each of R₁ through R₄ is H; or one of R₁ through R₄ is selected from the group consisting of CH₃, CH₂(OH), and CH(OH)₂, and each of the remaining three of R₁ through R₄ is H or OH.
18. The compound according to any one of the preceding embodiments, wherein n₁ and n₂ are each independently selected from an integer ranging from 0 to 3.
19. The compound according to any one of the preceding embodiments, wherein n₁ is selected from 0, 1, 2 or 3; and n₂ is selected from 0, 1 or 2.
20. The compound according to embodiment 19, wherein n₁ is 0; and n₂ is selected from 1 or 2.
21. The compound according to embodiment 19, wherein n₁ is 1; and n₂ is selected from 1 or 2.
22. The compound according to embodiment 19, wherein n₁ is 2; and n₂ is selected from 1 or 2.
23. The compound according to embodiment 19, wherein n₁ is selected from 1 or 2; and n₂ is 0.
24. The compound according to embodiment 19, wherein n₁ is selected from 1 or 2; and n₂ is 1.
25. The compound according to embodiment 19, wherein n₁ is selected from 1 or 2; and n₂ is 2.
26. The compound according to embodiment 19, wherein n₁ is 3; and n₂ is 1.
27. The compound according to embodiment 19, wherein n₁ is 2; and n₂ is 0.
28. The compound according to any one of the preceding embodiments, wherein L is selected from the group consisting of CH₂, O, S, SO, SO₂ and CH(OH).
29. The compound according to any one of embodiments 1 to 16, wherein (i) each of R₁ through R₄ is H; (ii) n₁ is selected from 0, 1 or 2; (iii) n₂ is selected from 1 or 2; (iv) L is selected from CH₂ and O; and (v) X₁ is O.
30. The compound according to any one of embodiments 1 to 16, wherein (i) each of R₁ through R₃ is H; (ii) R₄ is H or OH; (iii) wherein each of n₁ and n₂ is selected from 1 or 2; (iv) L is selected from CH₂, O and CH(OH); and (v) X₁ is O.
31. The compound according to any one of embodiments 1 to 16, wherein (i) each of R₁ through R₄ is H; (ii) n₁ is selected from 1, 2 or 3; (iii) n₂ is selected from 0, 1 or 2; (iii) L is selected from S, SO and SO₂; and (iv) X₁ is CH_{2.}
32. An RNA molecule whose 5′ end comprises a compound according to any one of embodiments 1 to 31.
33. An in vitro method for synthesizing an RNA molecule, the method comprising reacting nucleotides, the compound according to any one of embodiments 1 to 31, and a DNA template in the presence of a DNA-dependent RNA polymerase under conditions suitable for the transcription of the DNA template into an RNA molecule by the DNA-dependent RNA polymerase.
34. An RNA molecule obtained by the in vitro method according to embodiment 33.
35. The RNA molecule according to embodiments 32 or 34, wherein the RNA molecule comprises at least one chemical modification.
36. The RNA molecule according to embodiment 35, wherein the at least one chemical modification is selected from the group consisting of a base modification, a sugar modification and a backbone modification.
37. The RNA molecule according to embodiment 35 or 36, wherein the at least one chemical modification is a base modification, wherein the base modification is preferably selected from the group consisting of pseudouridine (psi or ψ), N1-methylpseudouracil (N1Mpsi or N1Mψ), 1-ethylpseudouracil, 2-thiouracil (s2U), 4-thiouracil, 5-methylcytosine, 5-methyluracil, 5-methoxyuracil, and any combination thereof.
38. The RNA molecule according to any one of embodiments 32 and 34 to 37, wherein the RNA molecule is a coding RNA comprising at least one coding sequence, preferably wherein the coding RNA is an mRNA.
39. The RNA molecule according to any one of embodiments 32 and 34 to 38, wherein the RNA molecule is a therapeutic mRNA.
40. A composition comprising the RNA molecule according to any one of embodiments 32 and 34 to 39.
41. The composition according to embodiment 40, wherein the composition is a pharmaceutical composition.
42. A kit comprising the compound according to any one of embodiments 10 to 31, and a DNA-dependent RNA polymerase.
43. The kit according to embodiment 42, wherein the kit further comprises nucleotides.
44. The kit according to embodiment 42 or 43, wherein the kit further comprises a ribonuclease inhibitor.
45. The kit according to any one of embodiments 42 to 44, wherein the kit further comprises a buffer.
46. Use of the compound according to any one of embodiments 1 to 31 in an in vitro transcription reaction for producing a capped RNA molecule.
47. The use according to embodiment 46, wherein the capped RNA molecule is the RNA molecule according to any one of embodiments 32 or 34 to 39.

A set of embodiments (C) of the present application relates to:
1. A compound of formula (I):

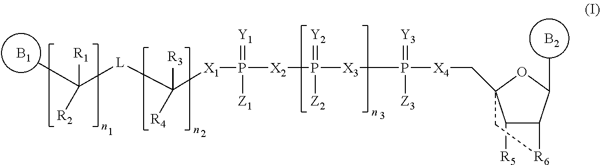

or a salt, stereoisomer, tautomer, or deuterated version thereof, wherein
R₅ is OH or

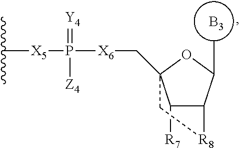

wherein
R₇ is OH or

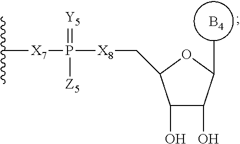

ring B₁ is guanine, a modified guanine, or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
n₃ is selected from 0, 1 or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
X₁ is CH₂, and each of X₂ through X₈ is independently O, S, NH or CH₂;
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) O, wherein the dashed methylene bridge between R₆ being 0 and the 4′ C is present;
R₈ is (i) selected from the group consisting of H, OH, OC₁-C₃-alkyl, and Opropargyl, wherein the dashed methylene bridge between R₃ and the 4′ C is absent, or (ii) O, wherein the dashed methylene bridge between R₈ being 0 and the 4′ C is present; and
each of ring B₂ through ring B₄ is independently a nucleobase, a modified nucleobase, or a nucleobase analog.
2. The compound according to embodiment 1, wherein
   n₃ is 1;
   R₆ is selected from the group consisting of H, OH, OC₁-C₃-alkyl, and Opropargyl, wherein the dashed methylene bridge between R₆ and the 4′ C is absent; and
   R₈ is selected from the group consisting of H, OH, OC₁-C₃-alkyl, and Opropargyl, wherein the dashed methylene bridge between R₈ and the 4′ C is absent.
3. The compound according to embodiment 1 or 2, wherein B₂ is selected from the group consisting of guanine, a modified guanine, a guanine analog, an adenine, a modified adenine, and an adenine analog.
4. The compound according to any one of the preceding embodiments, wherein B₃ is guanine, a modified guanine, or a guanine analog.
5. The compound according to any one of the preceding embodiments, wherein R₅ is OH and wherein R₆ is OH, wherein the dashed methylene bridge between R₆ and the 4′ C is absent.
6. The compound according to any one of embodiments 1 to 4, wherein R₅ is

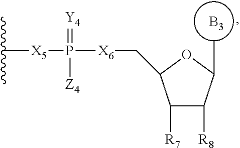

wherein R₇ and R₈ are each OH.
7. The compound according to embodiment 6, wherein R₆ is H or OC₁-C₃-alkyl, preferably wherein R₆ is OCH_{3.}
8. The compound according to any one of embodiments 1 to 4, wherein R₅ is

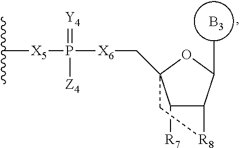

wherein R₇ is

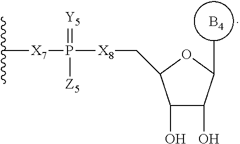

9. The compound according to embodiment 8, wherein
   R₆ is H or OC₁-C₃-alkyl, wherein the dashed methylene bridge between R₆ and the 4′ C is absent, preferably wherein R₆ is OCH₃; and/or
   R₈ is H or OC₁-C₃-alkyl, wherein the dashed methylene bridge between R₈ and the 4′ C is absent, preferably wherein R₈ is OCH_{3.}
10. The compound according to any one of the preceding embodiments, wherein ring B₁ is a modified guanine.
11. The compound according to any one of the preceding embodiments, wherein ring B₁ is N⁷-methylguanine.
12. The compound according to any one of the preceding embodiments, wherein each of X₂ through X₈ is O.
13. The compound according to any one of the preceding embodiments, wherein each of Y₁ through Y₅ is O.
14. The compound according to any one of the preceding embodiments, wherein each of Z₁ through Z₅ is OH.
15. The compound according to any one of the preceding embodiments, wherein each of R₁ through R₄ is independently H or OH; or one of R₁ through R₄ is selected from the group consisting of CH₃, CH₂(OH), and CH(OH)₂, and each of the remaining three of R₁ through R₄ is independently H or OH.
16. The compound according to any one of the preceding embodiments, wherein each of R₁ through R₃ is H and R₄ is H or OH.
17. The compound according to any one of the preceding embodiments, wherein each of R₁ through R₄ is H; or one of R₁ through R₄ is selected from the group consisting of CH₃, CH₂(OH), and CH(OH)₂, and each of the remaining three of R₁ through R₄ is H or OH.
18. The compound according to any one of the preceding embodiments, wherein n₁ and n₂ are each independently selected from an integer ranging from 0 to 3.
19. The compound according to any one of the preceding embodiments, wherein n₁ is selected from 0, 1, 2 or 3; and n₂ is selected from 0, 1 or 2.
20. The compound according to embodiment 19, wherein n₁ is 0; and n₂ is selected from 1 or 2.
21. The compound according to embodiment 19, wherein n₁ is 1; and n₂ is selected from 1 or 2.
22. The compound according to embodiment 19, wherein n₁ is 2; and n₂ is selected from 1 or 2.
23. The compound according to embodiment 19, wherein n₁ is selected from 1 or 2; and n₂ is 0.
24. The compound according to embodiment 19, wherein n₁ is selected from 1 or 2; and n₂ is 1.
25. The compound according to embodiment 19, wherein n₁ is selected from 1 or 2; and n₂ is 2.
26. The compound according to embodiment 19, wherein n₁ is 3; and n₂ is 1.
27. The compound according to embodiment 19, wherein n₁ is 2; and n₂ is 0.
28. The compound according to any one of the preceding embodiments, wherein L is selected from the group consisting of CH₂, O, S, SO, SO₂ and CH(OH).
29. The compound according to any one of embodiments 1 to 16, wherein (i) each of R₁ through R₄ is H; (ii) n₁ is selected from 1, 2 or 3; (iii) n₂ is selected from 0, 1 or 2; (iii) L is selected from S, SO and SO₂; and (iv) X₁ is CH_{2.}
30. An RNA molecule whose 5′ end comprises a compound according to any one of embodiments 1 to 29.
31. An in vitro method for synthesizing an RNA molecule, the method comprising reacting nucleotides, the compound according to any one of embodiments 1 to 29, and a DNA template in the presence of a DNA-dependent RNA polymerase under conditions suitable for the transcription of the DNA template into an RNA molecule by the DNA-dependent RNA polymerase.
32. An RNA molecule obtained by the in vitro method according to embodiment 31.
33. The RNA molecule according to embodiments 30 or 32, wherein the RNA molecule comprises at least one chemical modification.
34. The RNA molecule according to embodiment 32, wherein the at least one chemical modification is selected from the group consisting of a base modification, a sugar modification and a backbone modification.
35. The RNA molecule according to embodiment 33 or 34, wherein the at least one chemical modification is a base modification, wherein the base modification is preferably selected from the group consisting of pseudouridine (psi or ty), N1-methylpseudouracil (N1Mpsi or N1Mψ), 1-ethylpseudouracil, 2-thiouracil (s2U), 4-thiouracil, 5-methylcytosine, 5-methyluracil, 5-methoxyuracil, and any combination thereof.
36. The RNA molecule according to any one of embodiments 30 and 32 to 35, wherein the RNA molecule is a coding RNA comprising at least one coding sequence, preferably wherein the coding RNA is an mRNA.
37. The RNA molecule according to any one of embodiments 30 and 32 to 36, wherein the RNA molecule is a therapeutic mRNA.
38. A composition comprising the RNA molecule according to any one of embodiments 30 and 32 to 37.
39. The composition according to embodiment 38, wherein the composition is a pharmaceutical composition.
40. A kit comprising the compound according to any one of embodiments 10 to 29, and a DNA-dependent RNA polymerase.
41. The kit according to embodiment 40, wherein the kit further comprises nucleotides.
42. The kit according to embodiment 40 or 41, wherein the kit further comprises a ribonuclease inhibitor.
43. The kit according to any one of embodiments 40 to 42, wherein the kit further comprises a buffer.
44. Use of the compound according to any one of embodiments 1 to 29 in an in vitro transcription reaction for producing a capped RNA molecule.
45. The use according to embodiment 44, wherein the capped RNA molecule is the RNA molecule according to any one of embodiments 30 or 32 to 37.

### EXAMPLES

In the following section, particular examples illustrating various embodiments and aspects of the invention are presented. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the claims as disclosed herein.

### Example 1: Synthesis of Compounds of the Present Invention

### Starting Materials, Methods and Analytical Data:

Unless otherwise specified, all starting materials are obtained from commercial suppliers or prepared by methods known to the skilled person. Unless otherwise specified, all reactions are conducted at RT. Otherwise, the temperatures are expressed as ° C. Solvent removal was carried out using Rotary evaporator if not otherwise specified. The conditions for column chromatography and HPLC are specified in each case. MS was conducted using an AmaZon SL (Bruker) with ESI ion source and ion trap analyzer or an Exploris 240 (Thermo Fisher) with HESI ion source and orbitrap analyzer. ESI-MS was carried in positive mode with Methanol+Formic acid or 5 mM ammoniumacetate+methanol or in negative mode with 5 mM ammoniumacetate+methanol as solvents. NMR spectra were recorded using a Bruker Avance III HDX 400 with a 5 mm BBFO sample head or an Magritek Ultra 80 MHz. Chemical shifts (δ) are reported in ppm relative to the residual solvent signal ¹H NMR data are reported as follows: chemical shift (multiplicity, coupling constants and number of hydrogens). Multiplicity is abbreviated as follows: s (singlet), d (doublet), t (triplet), q (quartet), m (multiplet), br (broad signal).

### LIST OF ABBREVIATIONS

| | |
|---|---|
| DCM | Dichloromethane |
| DIAD | Diisopropyl-azodicarboxylat |
| DMF | Dimethylformamide |
| DMSO | Dimethylsulfoxide |
| EDTA | Ethylenediaminetetraacetic acid |
| EtOH | Ethanol |
| MCPBA | meta-Chloroperoxybenzoic acid |
| MeCN | Acetonitrile |
| MeOH | Methanol |
| TEAB | Triethylammonium bicarbonate |
| TFA | Trifluoroacetic acid |
| THF | Tetrahydrofuran |
| TLC | Thin layer chromatography |

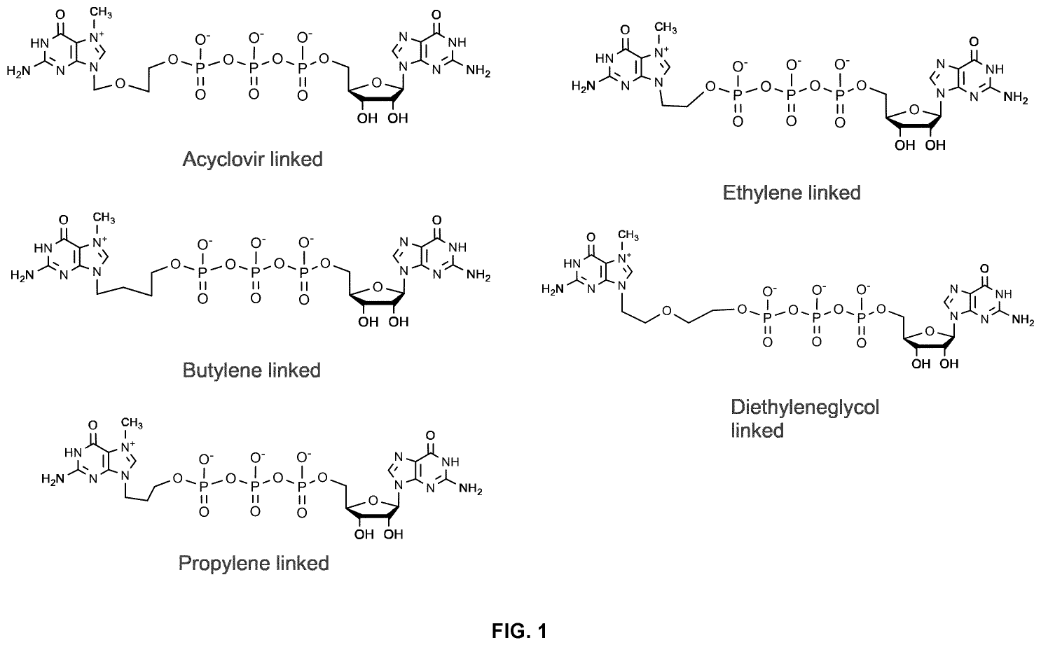

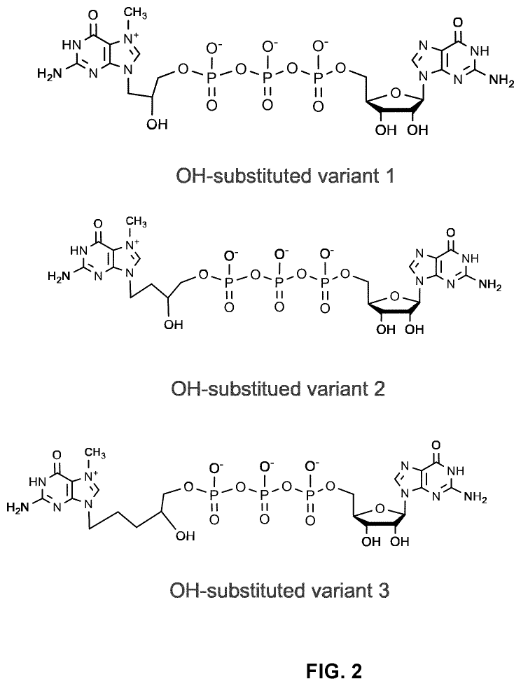

Synthesis route I as described below in Example 1.1 results in m7Guanin-9-butyl-ppp-(5′)G (which is referred to in FIG. 1 as “Butylene linked” compound). Other linear ribose variants with phosphate moieties can be prepared accordingly. Synthesis route 11 as described below in Example 1.2 discloses the synthesis of m7Guanin-9-(2-Hydroxypropyl)-ppp(5′)G (which is referred to in FIG. 2 as “OH-substituted variant 1”) and other hydroxy substituted variants may be produced accordingly. Synthesis route III as described below in Example 1.3. provides variants in three different oxidation states, namely m7Guanin-9-(propyl-thio-methyl)-ppp(5′)G, m7Guanin-9-(propyl-sulfinyl-methyl)-ppp(5′)G and m7Guanin-9-(propyl-sulfonyl-methyl)-ppp(5′)G. Route Ill may generally be used when synthesizing phosphonate variants. Example 1.4. describes the synthesis of m7Guanin-9-(ethoxymethyl)-ppp(5′)G (which is referred to as “Acyclovir linked” compound in FIG. 1) according to synthesis route I.

### Example 1.1: Synthesis Route I

The synthesis of compound 5 (corresponding to m7Guanin-9-butyl-ppp-(5′)G referred to in FIG. 1 as “Butylene linked” compound) when starting from 1,4-butanediol is shown in the following. It is noted that the synthesis route when starting from other diol compounds (such as e.g. ethylene glycol, thiodiglycol or diethylene glycol) is identical. Examples of resulting compounds analogous to compound 5 when starting from such other diol compounds are described at the end of the present example and are depicted in FIG. 1, and the synthesis of the acyclovir-linked cap analog (compound 19, referred to as “Acyclovir linked” compound in FIG. 1) in accordance with synthesis route I is described in Example 1.4 below.

Synthesis of Compound 1 with the Following Structure:

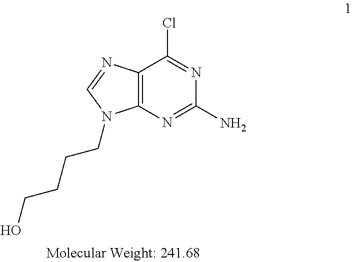 [SMILES: Nc1nc(Cl)c2ncn(CCCCO)c2n1]

1,4-Butanediole (0.35 g, 1.0 eq.) was dissolved in 42 mL dry THF and triphenylphosphine (2 eq.), and 2-amino-6-chloropurine (2 eq.) was added. DIAD was added (2 eq.) over a time of 5 minutes and the reaction mixture was stirred for 24 h under protective gas atmosphere. The reaction mixture was concentrated in vacuo and purified via flash column chromatography (Silica: 100 g, DCM/MeOH, 95:5 (500 mL), 97:3 (2500 mL), 90:10 (1000 mL) (v/v)). The product was obtained as white powder in a yield of 32%. ¹H NMR (400 MHz, DMSO-d6), δ 8.14 (s, 1H), 6.90 (s, 2H), 4.43 (t, 1H), 4.05 (t, 2H), 3.40 (q, 2H), 1.80 (m, 2H), 1.38 (m, 2H).

Synthesis of Compound 2 with the Following Structure:

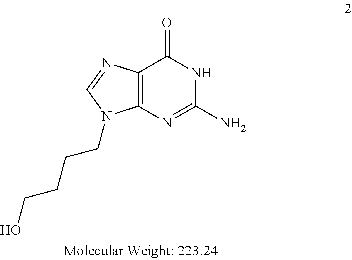 [SMILES: Nc1nc2c(ncn2CCCCO)c(=O)[nH]1]

Aqueous trifluoroacetic acid (7.5 mL TFA/water 3:1 per mmol of 1) was added to 1 (1.77 g) and the mixture was stirred for 72 h hours at room temperature. The reaction was stopped by evaporation of solvent and coevaporation with toluene (3×25 mL). The residue was purified by flash column chromatography (Silica: 100 g, step gradient: DCM/MeOH, 90:10 (500 mL), 85:15 (300 mL), 80:10 (300 mL), 70:30 (300 mL), 50:50 (500 mL) (v/v)). The product was obtained as white powder in a yield of 71%. ¹H NMR (400 MHz, DMSO-d6) δ 10.64 (s, 1H), 7.70 (s, 1H), 6.54 (s, 2H), 4.46 (br, 1H), 3.93 (t, 2H), 3.39 (t, 2H), 1.74 (m, 2H), 1.36 (m, 2H).

Synthesis of Compound 3 with the Following Structure:

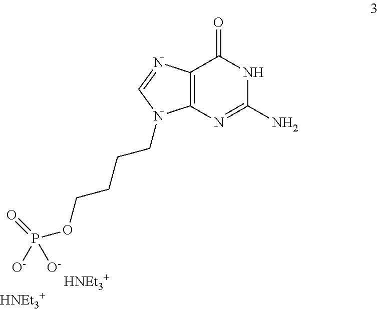 [SMILES: Nc1nc2c(ncn2CCCCOP(=O)([O-])[O-])c(=O)[nH]1]

Trimethylphosphate (13 eq.), proton sponge (2 eq.) and POCl₃ (2 eq.) precooled at 0° C. were added in a dry schlenk flask. Compound 2 (1 eq.) was added at once and the mixture was stirred for 4 h at 0° C. under protective gas atmosphere. The reaction was quenched after full conversion with TEAB-buffer (15 mL, 1 M, pH 8.5), diluted with 800 mL H2 O and adjusted pH to 7.0 with aqueous ammonia solution. The purification was carried out by ion exchange chromatography with Macro-Prep High Q resin using a TEAB gradient from 0 mM to 1 M and the solvent was evaporated from product fractions. The product was obtained in the form of the triethylammonium salt and directly used in next step.

Synthesis of Compound 4 with the Following Structure:

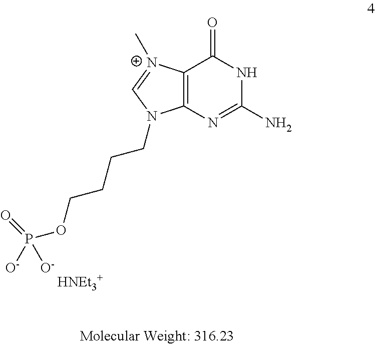 [SMILES: C[n+]1cn(CCCCOP(=O)([O-])[O-])c2nc(N)[nH]c(=O)c21]

The triethylammonium salt of compound 3 (1 eq.) was dissolved in DMSO resulting in a 0.1 M solution. Methyliodide (8 eq.) was added and reaction mixture was stirred for 6 h until the starting material was fully converted. 10 volume equivalents of water were added and the excess of methyl iodide was extracted with diethyl ether (4×). Residual methyl iodide in aqueous phase was reduced by addition of a small amount of sodium disulfite and the pH was adjusted to 7.0. The purification was carried out by ion exchange chromatography with Macro-Prep High Q resin using a TEAB gradient from 0 mM to 1 M. The solvent was evaporated of the product fractions. The product fraction was repurified via C18 RP HPLC, using a Phenomexx Gemini C18, 250×21.2 mm, 5 μM Column. Buffer A: 5 mM ammonium acetate pH 5.6, Buffer B: 100% MeOH, gradient program from Table Ex-1.

TABLE Ex-1

| Time total | Flow [ml/min] | % B |
|---|---|---|
| 0 | 6 | 0 |
| 25 | 6 | 12.5 |
| 26 | 6 | 80 |
| 30 | 6 | 80 |
| 31 | 6 | 0 |
| 35 | 6 | 0 |

The product fractions were lyophilized and analyzed via NMR. ¹H NMR (400 MHz, D2O) δ 4.27 (t, 2H), 4.09 (s, 3H), 3.89 (t, 2H), 2.01 (m, 2H), 1.67 (m, 2H).

If the following synthesis was carried out according to method A, the pooled product was transformed into triethylammonium salt via ion exchange resin (DOWEX 50W-X8 in triethylammonium form). The product was obtained as white solid in a yield of 62%.

Synthesis of Compound 5 with the Following Structure:

### Method A:

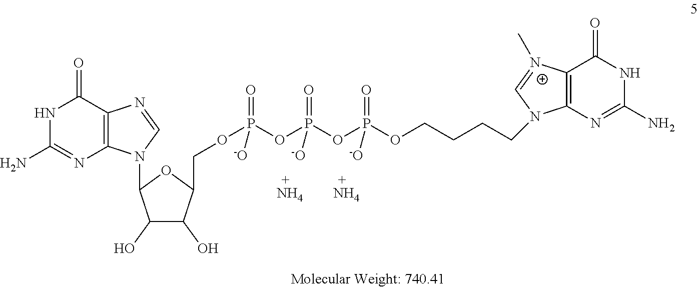

Compound 4 (1 eq.) was dissolved under argon atmosphere in a dry schlenk flask in dry DMF or in dry DMSO. Guanosine 5′-imidazolide diphosphate (1 eq.) synthesized according to [A. R. Kore, M. Shanmugasundaram, Current Protocols 2013, 55, 13.13.1-13.13.12] was added under argon atmosphere. Water-free zinc chloride (10 eq.) or water free magnesium chloride (10 eq.) was added and the reaction mixture was stirred for 16 h under argon atmosphere. The reaction was quenched by addition of EDTA (11 eq.) and adjusted to pH 7.0 with aqueous ammonia solution. The purification was carried out by ion exchange chromatography with Macro-Prep High Q resin using a TEAB gradient from 0 mM to 1 M and the solvent was evaporated of product fractions. The product fraction was repurified via C18 RP HPLC, using a Phenomexx Gemini C18, 250×21.2 mm, 5 μM Column. Buffer A: 5 mM ammonium acetate pH 5.6, Buffer B: 100% MeOH, gradient program in Table Ex-2. The product was obtained as white solid in a yield of 21%.

¹H NMR (400 MHz, D₂O) δ 8.88 (s, 1H), 8.17 (s, 1H), 5.86 (d, 1H), 4.67 (t, 1H), 4.48 (t, 1H), 4.35 (m, 1H), 4.30 (m, 1H), 4.26 (m, 1H), 4.12 (t, 2H), 4.06 (t, 2H), 4.04 (s, 3H), 1.95 (m, 2H), 1.69 (m, 2H).

TABLE Ex-2

| Time total | Flow [ml/min] | % B |
|---|---|---|
| 0 | 6 | 0 |
| 25 | 6 | 12.5 |
| 26 | 6 | 80 |
| 30 | 6 | 80 |
| 31 | 6 | 0 |
| 35 | 6 | 0 |

The product fractions were lyophilized and dissolved in ultrapure water resulting in a 100 mM solution.

Similar compounds can be synthesized in accordance with the above synthesis route when starting from other diol compounds. Exemplary compounds along these lines are shown in FIG. 1, namely i) the “Propylene linked” compound when starting from propanediole; ii) the “Ethylene linked” compound when starting from ethanediol; and iii) the “Diethyleneglycol linked” compound when starting from diethylene glycol.

### Method B

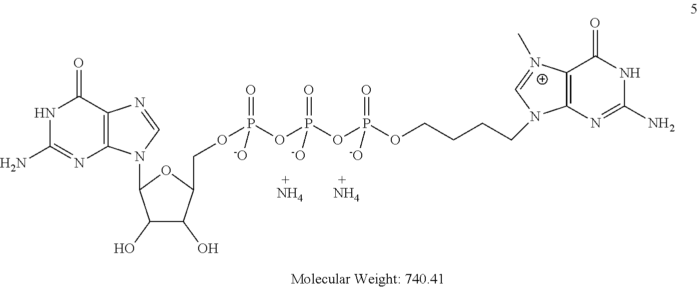

Compound 4 (1 eq.) was dissolved in water/acetonitrile (1:1, v/v) or in pure water or in aqueous N-Methylmorpholine (0.2M pH 7.0). Guanosine 5-imidazolide diphosphate (1.5 eq.) synthesized according to [A. R. Kore, M. Shanmugasundaram, Current Protocols 2013, 55, 13.13.1-13.13.12] was added. Magnesium chloride or zinc chloride or manganese chloride (10 eq.) was added and the reaction mixture was stirred for 16-24 h under argon atmosphere. The reaction was quenched by addition of EDTA (11 eq.) and adjusted to pH 7.0 with aqueous ammonia solution. The reaction mixture was desalted using RP-HPLC using the gradient program in table Ex-11 with solvent A: 5 mM ammonium acetate in water; solvent B: 80% methanol in water.

TABLE Ex-11

| Time [min] | A [%] | B [%] |
|---|---|---|
| 0 | 100 | 0 |
| 10 | 100 | 0 |
| 15 | 20 | 80 |
| 24 | 20 | 80 |
| 25 | 100 | 0 |
| 30 | 100 | 0 |

The desalted reaction mixture was purified by ion exchange chromatography with DNAPac PA200 column (22×250 mm) using Buffer A: 20 mM Tris, pH9, Buffer B: 20 mM Tris, 33 mM sodium perchlorate. The gradient program is described in table Ex-12.

TABLE Ex-12

| Time [min] | A [%] | B [%] |
|---|---|---|
| 0 | 100 | 0 |
| 30 | 25 | 75 |
| 31 | 0 | 100 |
| 35 | 0 | 100 |
| 36 | 100 | 0 |
| 40 | 100 | 0 |

The product fraction was desalted using RP-HPLC using the gradient program described in Table Ex-8 The resulting fraction was repurified via C18 RP HPLC, using a Phenomexx Gemini C18, 250×21.2 mm, 5 μM Column. Buffer A: 5 mM ammonium acetate pH 5.6, Buffer B: 100% MeOH, gradient program in Table Ex-10. The product was obtained as white solid in a yield of 13%.

¹H NMR (400 MHz, D₂O) δ 8.87 (s, 1H), 8.01 (s, 1H), 5.80 (d, 1H), 4.67 (t, 1H), 4.48 (t, 1H), 4.35 (m, 1H), 4.27 (m, 2H), 4.10 (t, 2H), 4.05 (t, 2H), 4.02 (s, 3H), 1.94 (m, 2H), 1.69 (m, 2H).

TABLE Ex-10

| Time [min] | A [%] | B [%] |
|---|---|---|
| 0 | 100 | 0 |
| 10 | 100 | 0 |
| 20 | 90 | 10 |
| 21 | 20 | 80 |
| 25 | 20 | 80 |
| 26 | 100 | 0 |
| 35 | 100 | 0 |

The product fractions were lyophilized and dissolved in ultrapure water resulting in a 100 mM solution.

Similar compounds can be synthesized in accordance with the above synthesis route when starting from other diol compounds. Exemplary compounds along these lines are shown in FIG. 1, namely i) the “Propylene linked” compound when starting from propanediole; ii) the “Ethylene linked” compound when starting from ethanediol; and iii) the “Diethyleneglycol linked” compound when starting from diethylene glycol.

### Example 1.2: Synthesis Route II

The synthesis of compound 8 (corresponding to m7Guanin-9-(2-Hydroxypropyl)-ppp(5′)G and referred to in FIG. 2 as “OH-substituted variant 1”) when starting from glycidol (epoxypropanol) is shown in the following. It is noted that the synthesis route when starting from other epoxides is identical. Examples of resulting compounds analogous to compound 8 when starting from such other epoxides are depicted in FIG. 2.

Synthesis of Compound 6 with the Structure Shown in the Following:

Synthesis of X₁ was performed according to literature: L Zhang, A. E. Peritz, P. J. Carroll, E. Meggers, Synthesis 2006, No. 4, 645-653, which involved the use of glycidol.

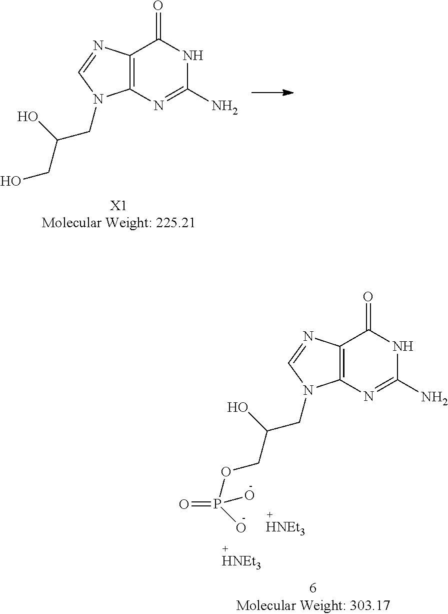 [SMILES: Nc1nc2c(ncn2CC(O)CO)c(=O)[nH]1 ; Nc1nc2c(ncn2CC(O)COP(=O)(O)O)c(=O)[nH]1]

Trimethylphosphate, (13 eq.), proton sponge (2 eq.) and POCl₃ (2 eq.) precooled at 0° C. were added to a dry schlenk flask. Compound X₁ (1 eq.) was added at once and the reaction was stirred for 4 h at 0° C. under protective gas atmosphere. The reaction was quenched after full conversion with TEAB-buffer (15 mL, 1 M, pH 8.5), diluted with 800 mL H₂O and the pH was adjusted to 7.0 with aqueous ammonia solution. Purification was carried out by ion exchange chromatography with Macro-Prep High Q resin using a TEAB gradient from 0 mM to 1 M and the solvent was evaporated from product fractions. The product fraction was repurified via C18 RP HPLC, using a Phenomexx Gemini C18, 250×21.2 mm, 5 μM Column. Buffer A: 5 mM ammonium acetate pH 5.6, Buffer B: 100% MeOH, gradient program in Table Ex-3.

TABLE Ex-3

| Time total | Flow [ml/min] | % B |
|---|---|---|
| 0 | 6 | 0 |
| 25 | 6 | 12.5 |
| 26 | 6 | 80 |
| 30 | 6 | 80 |
| 31 | 6 | 0 |
| 35 | 6 | 0 |

The product fractions were lyophilized and the resulting product was transformed for subsequent cap synthesis according to method A (as described in Example 1.1.) into triethylammonium salt via ion exchange resin DOWEX 50W-X8 (triethylammonium form). The product was obtained as triethylammonium salt in a yield of 21%.

¹H NMR (400 MHz, D₂O) δ 7.79 (s, 1H), 4.21 (dd, 1H), 4.18 (m, 1H), 4.11 (m, 1H), 3.92 (m, 2H).

Synthesis of Compounds 7 and 8 with the Following Structures:

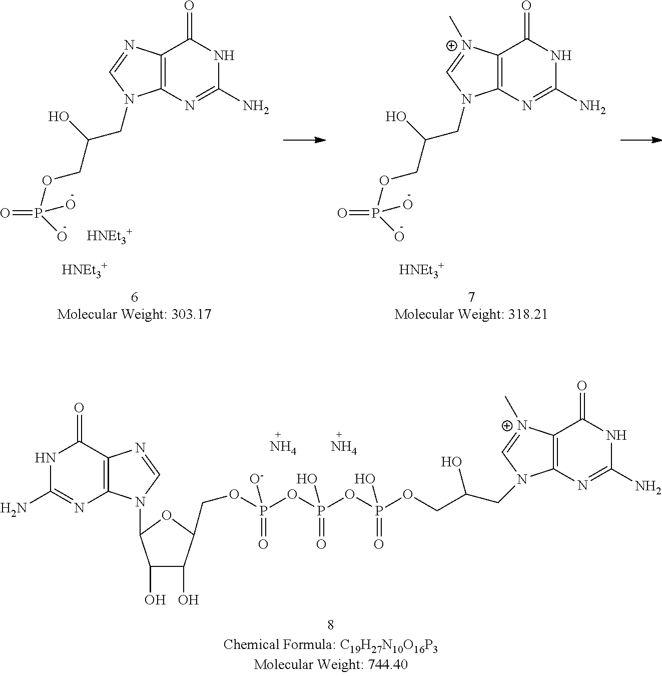 [SMILES: Nc1nc2c(ncn2CC(O)COP(=O)([O-])[O-])c(=O)[nH]1 ; C[n+]1cn(CC(O)COP(=O)([O-])[O-])c2nc(N)[nH]c(=O)c21 ; C[n+]1cn(CC(O)COP(=O)(O)OP(=O)(O)OP(=O)([O-])OCC2OC(n3cnc4c(=O)[nH]c(N)nc43)C(O)C2O)c2nc(N)[nH]c(=O)c21 ; [NH4+] ; [NH4+]]

Starting from compound 6, the N7-methylation via methyl iodide to compound 7 and the following zinc or magnesium mediated condensation reaction to compound 8 were performed according to the protocols described above for the synthesis of compound 4 and compound 5.

ESI-MS: [M+H]: 745.089, Calculated 745.089.

Similar compounds can be synthesized in accordance with the above synthesis route when starting from other epoxides in order to provide the starting compound X. Exemplary compounds along these lines are shown in FIG. 2, namely i) the “OH-substituted variant 2” compound when starting from epoxybutanol; and ii) the “OH-substituted variant 3” compound when starting from epoxypentanol.

### Example 1.3: Synthesis Route III

The synthesis of compound 12 (corresponding to m7Guanin-9-(propyl-sulfonyl-methyl)-ppp(5′)G), compound 14 (corresponding to m7Guanin-9-(propyl-thio-methyl)-ppp(5′)G) and compound 16 (corresponding to m7Guanin-9-(propyl-sulfinyl-methyl)-ppp(5′)G) when starting from three different precursors, namely precursors in three different oxidation states, is shown in the following. It is noted that the synthesis route when starting from precursors with different chain lengths of the carbon-linker and/or modifications in this carbon-linker is identical. Synthesis of precursors X2, X3 and X4 was performed according to the literature: T. Klejch and D. Hockova, Eur. J. of Med. Chem. 183 (2019) 111667 PMID: 31536893. The structures of these precursors are as follows, and it is to be understood that they may also be present and used in the form of their salts:

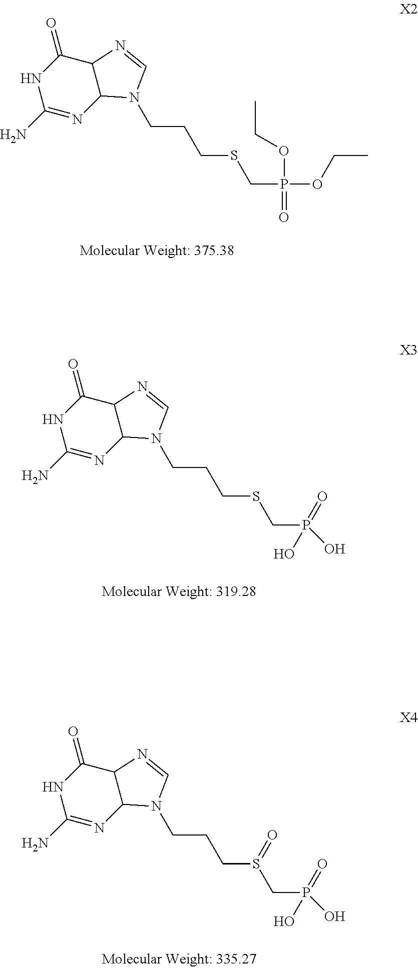 [SMILES: CCOP(=O)(CSCCCN1C=NC2C(=O)NC(N)=NC21)OCC ; NC1=NC2C(N=CN2CCCSCP(=O)(O)O)C(=O)N1 ; NC1=NC2C(N=CN2CCCS(=O)CP(=O)(O)O)C(=O)N1]

Synthesis of Compound 9 with the Following Structure:

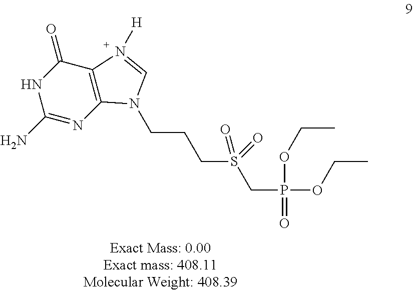

Compound X2 (1 eq.) was dissolved in dry DCM. The solution was cooled to 0° C. and MCPBA (3 eq.) was added. The reaction mixture was stirred for 2 h under protective gas atmosphere. After completion of the reaction, the solvent was evaporated and the residue was purified by flash chromatography (Silica, Ethylacetate/Methanol gradient according to Table Ex-4), yielding in a white solid 69% yield). ¹H NMR (400 MHz, DMSO-d₆) δ 10.58 (s, 1H), 7.70 (s, 1H), 6.48 (s, 2H), 4.27 (d, 2H), 4.07 (m, 6H), 3.29 (t, 2H), 2.20 (m, 2H), 1.24 (t, 6H).

ESI-MS: 408.09

TABLE Ex-4

| Step | Time [min] | % 2^{nd} solvent (MeOH) |
|---|---|---|
| 1 | 0 | 0 |
| 2 | 3 | 0 |
| 3 | 25 | 50 |
| 4 | 10 | 50 |
| 5 | 3 | 100 |
| 6 | 5 | 100 |

Synthesis of Compound 10 with the Following Structure:

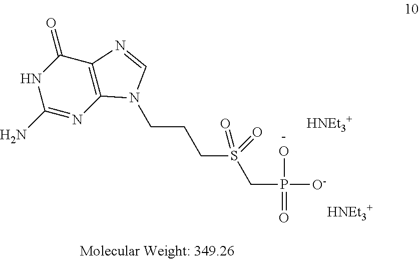 [SMILES: Nc1nc2c(ncn2CCCS(=O)(=O)CP(=O)([O-])O)c(=O)[nH]1]

Compound 9 (1 eq.) was dissolved/suspended in dry dichloromethane under an argon atmosphere. Afterwards, trimethylsilyl bromide (7.5 eq.) was added dropwise. The reaction mixture was stirred overnight under argon atmosphere. After completion, the reaction mixture was quenched by addition of methanol and stirring for 5 min. The solvent was evaporated. The residue was dissolved in MeOH/H₂O (1:1, v/v), stirred for 30 min and the solvent was evaporated. The residue was dissolved in water and purified by ion exchange chromatography with Macro-Prep High Q resin using a TEAB gradient from 0 mM to 1 M. Solvent of product fractions was removed in vacuo. The product was obtained as white solid in a yield of 60%. ¹H NMR (400 MHz, D₂O) δ 8.96 (s, 1H), 4.45 (t, 2H), 3.97 (m, 2H), 3.76 (d, 2H), 3.48 (t, 2H), 2.47 (m, 2H).

Synthesis of Compounds 11 and 12 with the Following Structures:

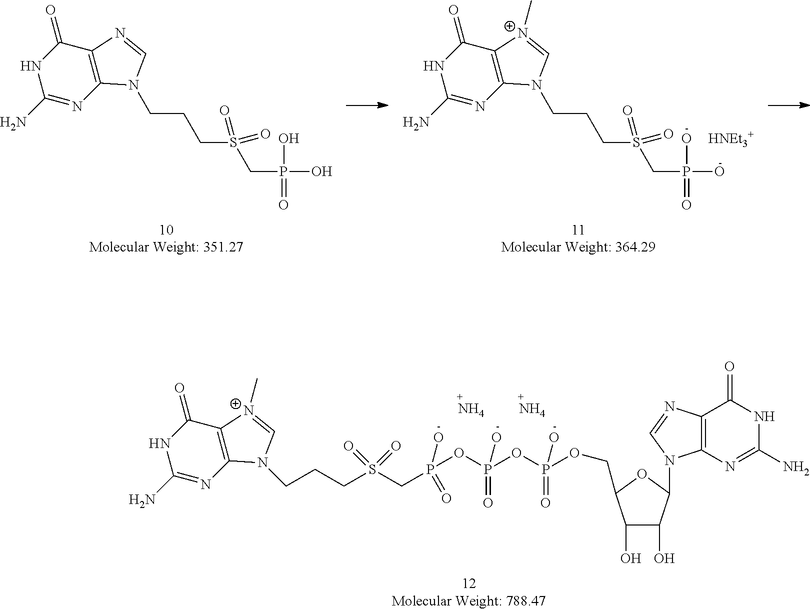

Starting from compound 10 transformed into triethylammonium salt via ion exchange resin DOWEX 50W-X8 (triethylammonium form), the N7-methylation via methyl iodide to compound 11 was performed according to the protocols described above for the synthesis of compound 4. The following zinc or magnesium mediated condensation reaction to compound 12 was performed according to the protocols described above for the synthesis of compound 5 yielding in 13% of a white powder.

¹H NMR (400 MHz, D₂O) δ 8.05 (s, 1H), 5.85 (d, 1H), 4.73 (t, 1H), 4.51 (m, 1H), 4.35 (m, 1H), 4.26 (m, 4H), 4.05 (s, 3H), 3.93 (m, 1H), 3.50 (t, 2H), 2.37 (m, 2H).

³¹P NMR (162 MHz, D₂O) δ−4.76 (d), −11.30 (d), −23.18 (m).

Synthesis of Compounds 13 and 14 with the Following Structures:

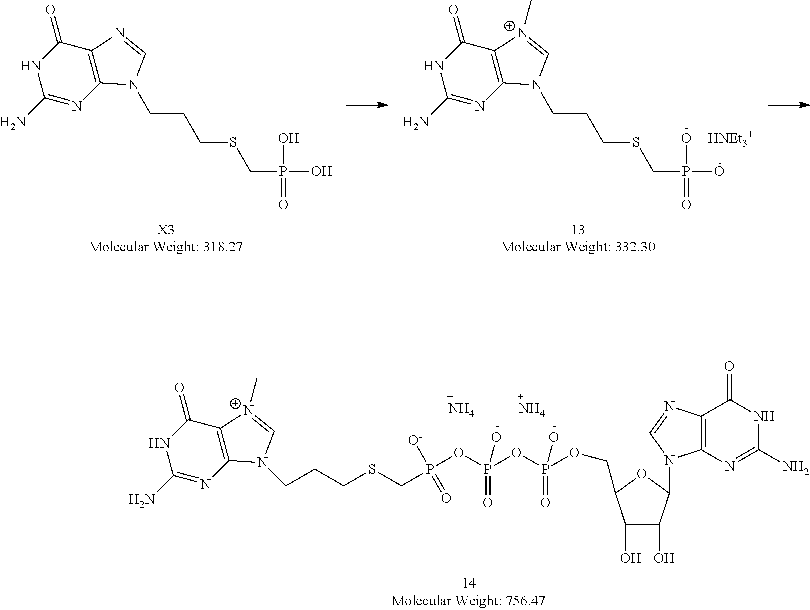

Starting from compound X3 transformed into triethylammonium salt via ion exchange resin DOWEX 50W-X8 (triethylammonium form), the N7-methylation via methyl iodide to compound 13 was performed according to the protocols described above for the synthesis of compound 4. The following zinc or magnesium mediated condensation reaction to compound 14 was performed according to the protocols described above for the synthesis of compound 5 yielding in 40% of a white powder.

¹H NMR (400 MHz, D₂O) δ 8.91 (s, 1H), 8.04 (s, 1H), 5.82 (d, 1H), 4.73 (t, 1H), 4.51 (m, 1H), 4.35 (m, 1H), 4.30 (m, 1H), 4.27 (m, 1H), 4.20 (m, 2H), 4.03 (s, 3H), 2.90 (d, 2H), 2.70 (t, 2H), 2.09 (m, 2H).

³¹P NMR (162 MHz, D₂O) δ 10.95 (d), −11.53 (d), −23.20 (m).

Synthesis of Compounds 15 and 16 with the Following Structures:

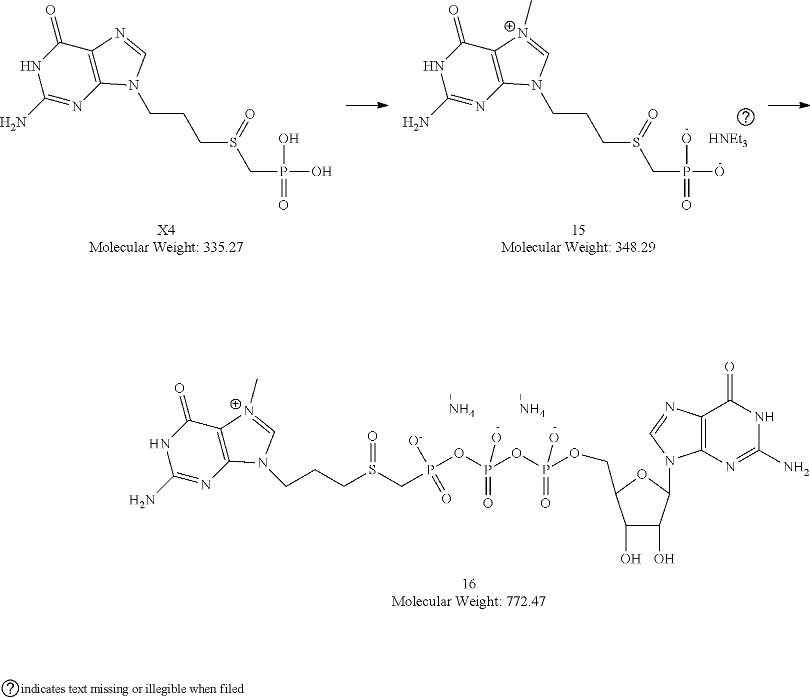 [SMILES: Nc1nc2c(ncn2CCCS(=O)CP(=O)(O)O)c(=O)[nH]1 ; C[n+]1cn(CCCS(=O)CP(=O)([O-])[O-])c2nc(N)[nH]c(=O)c21 ; C[n+]1cn(CCCS(=O)CP(=O)([O-])OP(=O)([O-])OP(=O)([O-])OCC2OC(n3cnc4c(=O)[nH]c(N)nc43)C(O)C2O)c2nc(N)[nH]c(=O)c21 ; [NH4+] ; [NH4+] ; "HNEt3"]

Starting from compound X4 transformed into triethylammonium salt via ion exchange resin DOWEX 50W-X₈ (triethylammonium form). The N7-methylation via methyl iodide to compound 15 was performed according to the protocols described above for the synthesis of compound 4. The following zinc or magnesium mediated condensation reaction to compound 16 was performed according to the protocols described above for the synthesis of compound 5 yielding in 10% of a white powder.

¹H NMR (400 MHz, D₂O) δ 8.04 (s, 1H), 5.83 (d, 1H), 4.69 (q, 1H), 4.51 (q, 1H), 4.36 (m, 1H), 4.28 (m, 4H), 4.03 (s, 3H), 3.64 (t, 1H). 3.49 (t, 1H), 3.28 (m, 1H), 3.07 (m, 1H).

³¹P NMR (162 MHz, D₂O) δ 0.39 (d), −11.45 (d), −23.19 (m).

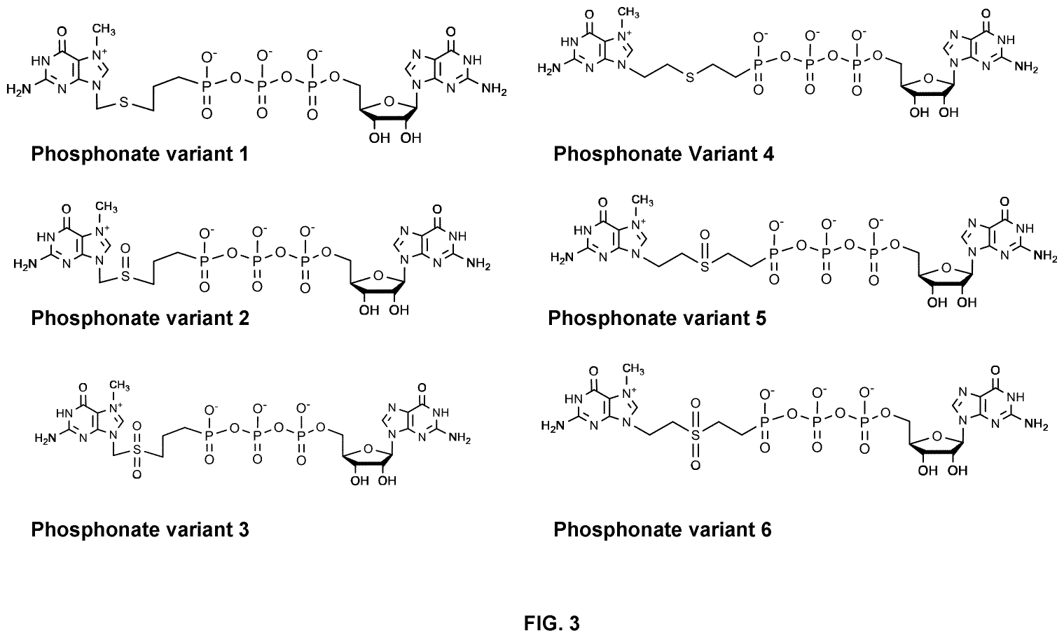

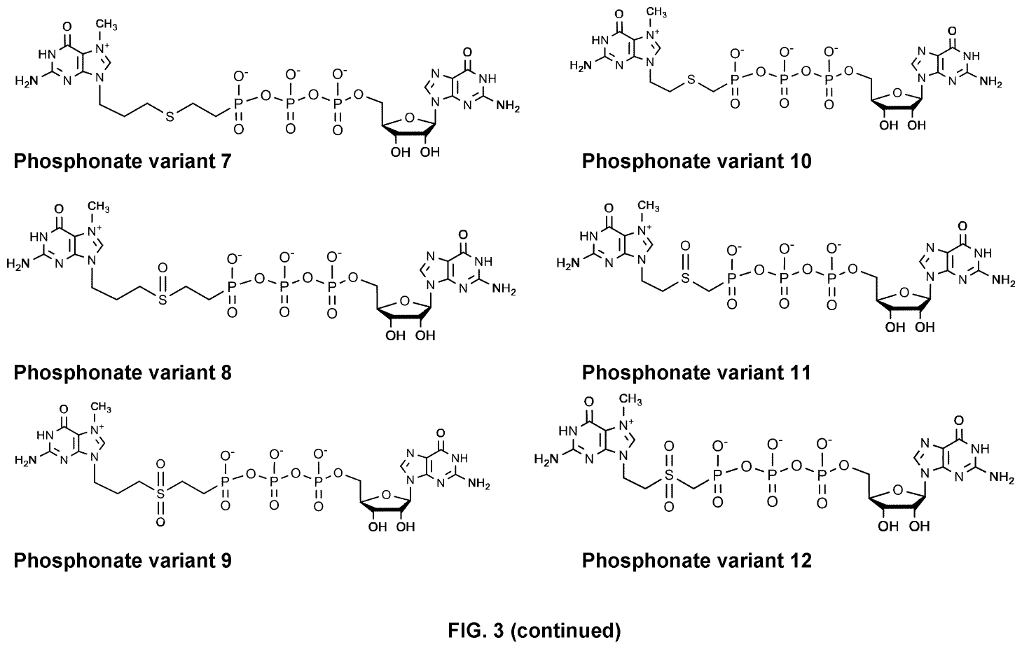

Similar compounds can be synthesized in accordance with the above synthesis route when starting from other starting compounds X, wherein the chain length of the carbon linker (and optionally the substitution pattern) is different from the length (and pattern) in compounds 12, 14 and 16 above. Examples of such variants are shown in FIG. 3.

### Example 1.4: Synthesis of the Acyclovir-Linked Cap Analog According to Synthesis Route I

The synthesis of the acyclovir-linked cap analog (compound 19 corresponding to XYZ) is shown in the following, wherein the synthesis inter alia starts from commercially available acyclovir (9-(2-Hydroxyethoxymethyl)-guanin, Carbosynth, UK) with the following structure, referred to herein as X5:

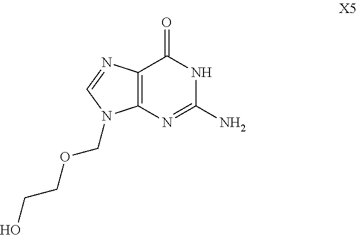 [SMILES: Nc1nc2c(ncn2COCCO)c(=O)[nH]1]

Synthesis of Compound 17 with the Following Structure:

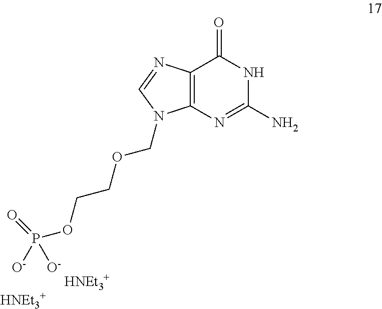 [SMILES: Nc1nc2c(ncn2COCCOP(=O)([O-])[O-])c(=O)[nH]1]

Trimethylphosphate (13 eq.), proton sponge (2 eq.) and POCl₃ (2 eq.) precooled at 0° C. were added to a dry schlenk flask. Acyclovir (X5) (1 eq.) was added at once and the mixture stirred for 6 h at 0° C. under protective gas atmosphere. The reaction after full conversion was quenched with TEAB-buffer (15 mL, 1 M, pH 8.5), diluted with 800 mL H₂O and the pH was adjusted to 7.0 with aqueous ammonia solution. Purification was carried out by ion exchange chromatography with Macro-Prep High Q resin using a TEAB gradient from 0 mM to 1 M, and the solvent was evaporated from product fractions.

Synthesis of Compound 18 with the Following Structure:

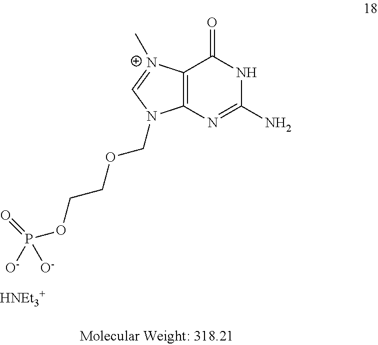 [SMILES: C[n+]1cn(COCCOP(=O)([O-])[O-])c2nc(N)[nH]c(=O)c21]

The triethyl ammonium salt of compound 17 (1 eq.) was dissolved in DMSO resulting in a 0.1 M solution. Methyliodide (8 eq.) was added and reaction mixture was stirred for 6 h until the starting material was fully converted according to TLC. 10 volumes of water were added and excess of methyl iodide was extracted with diethyl ether (4×). Residual methyl iodide in aqueous phase was reduced by addition of a small amount of sodium disulfite and the pH was adjusted to 7.0. Purification was carried out by ion exchange chromatography with Macro-Prep High Q resin using a TEAB gradient from 0 mM to 1 M and the solvent was evaporated of product fractions. The product fraction was repurified via C18 RP HPLC, using a Phenomexx Gemini C18, 250×21.2 mm, 5 μM Column. Buffer A: 5 mM ammonium acetate pH 5.6, Buffer B: 100% MeOH, gradient program from Table Ex-1 above. The product fractions were lyophilized and the resulting product was transformed for the subsequent cap synthesis according to method A into triethylammonium salt via ion exchange resin DOWEX 50W-X₈ (triethylammonium form). The product was obtained as white solid in a yield of 68%. ¹H NMR (400 MHz, DMSO-d₆) δ 5.71 (s, 2H), 4.11 (s, 3H), 3.95 (m, 2H), 3.86 (m, 2H).

Synthesis of Compound 19 with the Following Structure:

### Method A

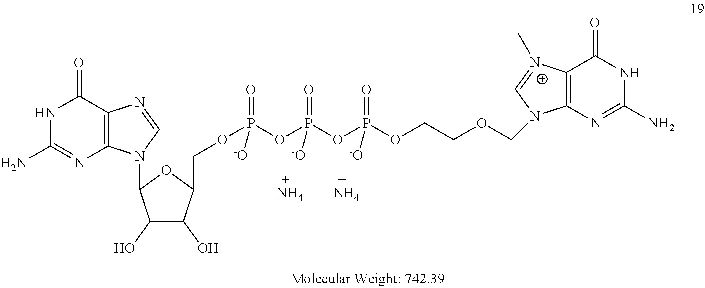

Compound 18 (1 eq.) was dissolved under argon atmosphere in a dry schlenk flask in dry DMF. Guanosine 5′-imidazolide diphosphate (1 eq.) synthesized according to [A. R. Kore, M. Shanmugasundaram, Current Protocols 2013, 55, 13.13.1-13.13.12] was added under argon atmosphere. Water-free zinc chloride (10 eq.) was added and the reaction mixture was stirred for 16 h under argon atmosphere. The reaction was quenched by addition of EDTA (11 eq.) and the pH was adjusted to 7.0 with aqueous ammonia solution. Purification was carried out by ion exchange chromatography with Macro-Prep High Q resin using a TEAB gradient from 0 mM to 1 M and the solvent was evaporated of product fractions. The product fraction was repurified via C18 RP HPLC, using a Phenomexx Gemini C18, 250×21.2 mm, 5 μM Column. Buffer A: 5 mM ammonium acetate pH 5.6, Buffer B: 100% MeOH, gradient program in above Table Ex-2. The product fractions were lyophilized and dissolved in ultrapure water resulting in a 100 mM solution. The product was obtained as white powder in a yield of 13%. ¹H NMR (400 MHz, DMSO-d₆) δ 7.95 (s, 1H), 5.77 (d, 1H), 5.49 (s, 2H), 4.61 (t, 1H), 4.43 (t, 1H), 4.28 (m, 1H), 4.20 (m, 2H), 4.06 (m, 2H), 3.99 (s, 3H), 3.76 (t, 2H).

Alternatively, compound 19 can be synthesized according to method B as described in Example 1.1.

### Example 1.5: Synthesis of the Gancyclovir-Linked Cap Analog According to Synthesis Route IV

The synthesis of the gancyclovir-linked cap analog (compound 20) is shown in the following, wherein the synthesis inter alia starts from commercially available gancyclovir (Carbosynth, UK) with the following structure, referred to herein as X5:

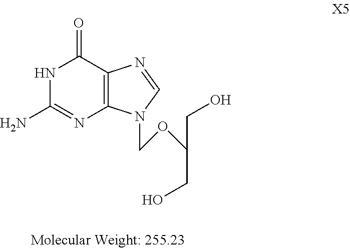 [SMILES: Nc1nc2c(ncn2COC(CO)CO)c(=O)[nH]1]

Synthesis of Compound 21 with the Following Structure:

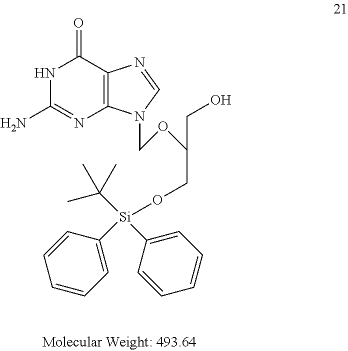 [SMILES: CC(C)(C)[Si](OCC(CO)OCn1cnc2c(=O)[nH]c(N)nc21)(c1ccccc1)c1ccccc1]

Gancyclovir X5 (1 eq.) was added to a dry Schlenk flask and dissolved in dry DMF.

Tertbutyl(chloro)diphenylsilane (1 eq.) and imidazole (1 eq.) was added and the reaction mixture was stirred for 12 h under protective gas atmosphere. Solvent was evaporated and crude product was purified by flash column chromatography (Silica: 120 g, linear gradient according to following table).

Gradient Table

| | Min | Solvents | % 2nd |
|---|---|---|---|
| 1 | 0.0 | AB | 0 |
| 2 | 26.7 | AB | 16 |
| 3 | 0.1 | AB | 16 |
| 4 | 1.6 | AB | 100 |
| 5 | 9.6 | AB | 100 |
| 6 | 5.0 | AB | 100 |

The product was obtained as white powder in a yield of 47%.

¹H NMR (80 MHz, DMSO-d6) δ 10.59 (s, 1H), 7.79 (s, 1H), 7.44 (m, 10H), 6.44 (s, 2H), 5.45 (s, 2H), 4.70 (t, 1H), 3.8-3.3 (br, 8H), 0.91 (s, 9H).

Synthesis of Compound 22 with the Following Structure:

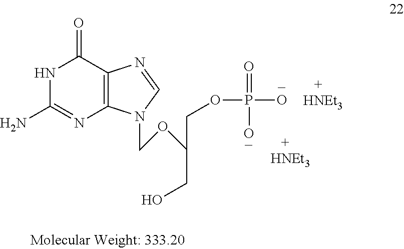 [SMILES: Nc1nc2c(ncn2COC(CO)COP(=O)(O)O)c(=O)[nH]1]

21 (1 eq.) was added to a dry schlenk flask and dissolved in extra dry acetonitrile for DNA synthesis. Bis-Cyanoethyl-N,N-diisopropyl-phosphoramidite (2 eq.) and Tetrazole (2 eq., 0.45M in acetonitrile) were added. The reaction mixture was stirred for 30 minutes. Oxidizer (0.1M Iodine in THE/Pyridine/water (77:21:2, v/v/v)) was added until red color was observed for 5 minutes. A 1:1 mixture of aqueous sodium disulfide solution (5 wt. %) and citric acid solution (5 wt. %) was added and crude product was washed with dichloromethane. The solvent was evaporated, and the residue was coevaporated with methanol. Concentrated ammonia solution was added and suspension was stirred at 60° C. for 48 h. Ammonia was removed by a steam of compressed air and solvent was evaporated. Crude product was dissolved in water and pH was adjusted to 7.0. Purification was carried out by ion exchange chromatography with Macro-Prep High Q resin using a TEAB gradient from 0 mM to 1 M and the solvent was evaporated of product fractions. The product fraction was repurified via C18 RP HPLC, using a Phenomexx Gemini C18, 250×21.2 mm, 5 μM Column. Buffer A: 5 mM ammonium acetate pH 5.6, Buffer B: 100% MeOH, gradient program from Table Ex-1 above.

ESI-MS: [M−H]: 334.0557, Calculated 334.0558.

¹H NMR (80 MHz, D₂O) δ 7.96 (s, 1H), 5.62 (s, 2H), 3.95 (m, 3H), 3.64 (s, 2H).

Synthesis of Compounds 23 and 20 with the Following Structures:

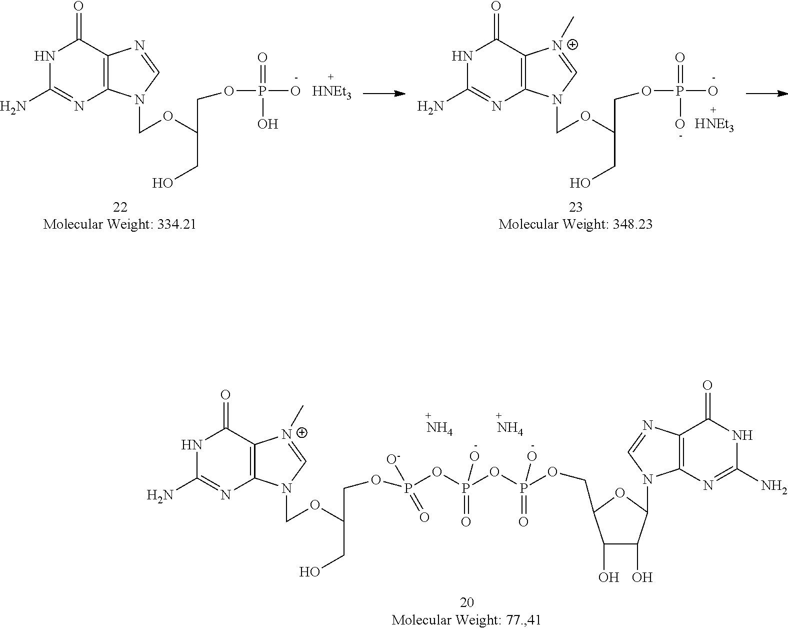

The N7-methylation via methyl iodide to compound 23 was performed according to the protocols described above for the synthesis of compound 4. The following zinc or magnesium mediated condensation reaction to compound 20 can be performed according to the protocols described above for the synthesis of compound 5.

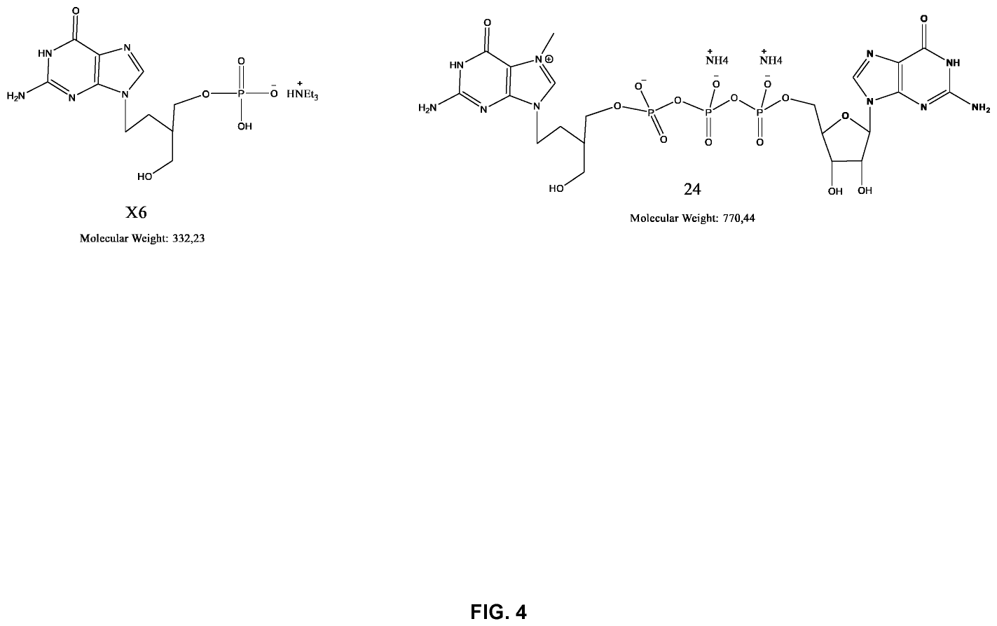

Similar compounds can be synthesized in accordance with the above synthesis route when starting from other starting compounds, e.g. X6, wherein the substitution pattern (and optionally the chain length of the carbon linker) is different from the pattern (and length) in compound 20. An example of such variants is shown in FIG. 4 FIG. 4.

### Example 1.6: Synthesis of a Propylene Linked Cap 1 Trinucleotide

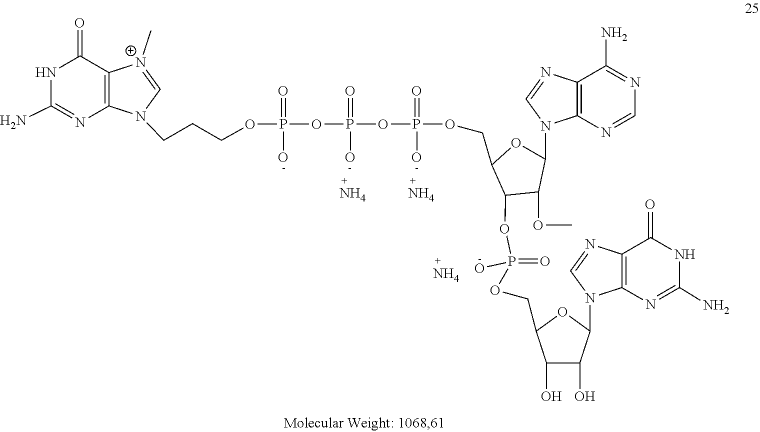

The synthesis of the propylene linked cap1 trinucleotide (compound 25) is shown in the following, wherein the synthesis inter alia started from commercially available 5′-O-DMT-N2-isobutyrylguanosine, Carbosynth, UK with the following structure, referred to herein as X7 and N6-Benzoyl-5′-O-DMT-2′-O-methyladenosine 3′-CE phosphoramidite, Carbosynth, UK) with the following structure, referred to herein as X8:

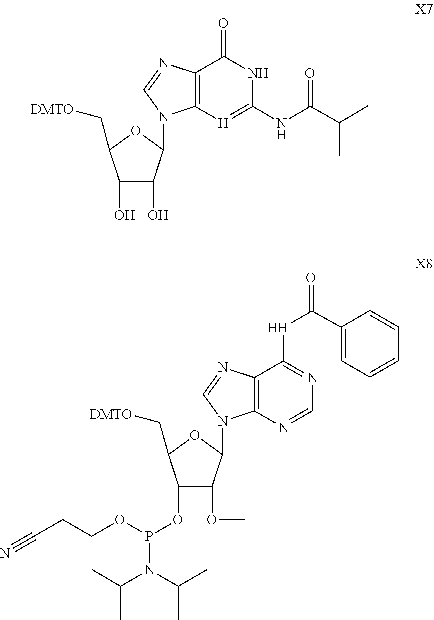

Synthesis of Compound 26 with the Following Structure:

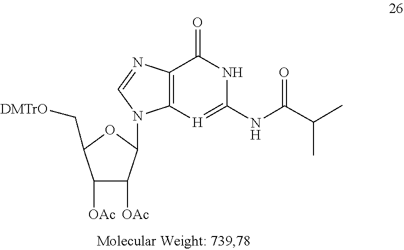

Compound X7 (1 eq.) was dissolved under argon atmosphere in dry pyridine. 4-(Dimethylamino)-pyridine (0.15 eq.) and acetic anhydride (14 eq.) were added and the reaction mixture was stirred for 6 h. Dichloromethane was added and organic phase was washed three times with aqueous citric acid (5 wt. % in water). Solvent was evaporated and crude product was purified by flash column chromatography (Silica: 330 g, Solvent A: Ethyl acetate, Solvent B: MeOH (2nd solvent), linear gradient according to following table:)

Gradient Table

| | Min | Solvents | % 2nd |
|---|---|---|---|
| 1 | 0.0 | AB | 0 |
| 2 | 29.0 | AB | 7 |
| 3 | 1.0 | AB | 80 |
| 4 | 10.0 | AB | 80 |

The product was obtained as pale white foam in a yield of 84%.

¹H NMR (400 MHz, DMSO-d6) δ 12.12 (s, 1H), 11.52 (s, 1H), 8.12 (s, 1H), 7.36 (d, 2H), 7.22 (m, 7H), 6.83 (t, 4H), 6.14 (d, 1H), 5.96 (t, 1H), 5.47 (dd, 1H), 4.27 (m, 1H), 3.72 (s, 6H), 3.53 (dd, 1H), 3.27 (dd, 1H), 2.75 (m, 1H), 2.11 (s, 3H), 2.04 (s, 3H), 1.14 (s, 3H), 1.12 (s, 3H).

Synthesis of Compound 27 with the Following Structure:

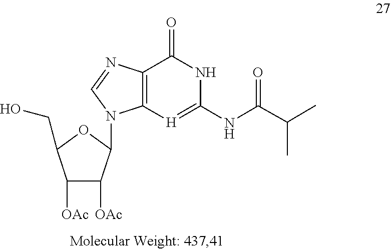

Compound 26 (1 eq.) was dissolved in aqueous acetic acid (80% acetic acid in water (v/v) and stirred for 30-60 minutes. Solvents were evaporated and residue was coevaporated with methanol. Crude product was purified by flash column chromatography (Silica: 120 g, Solvent A: Ethyl acetate, solvent B (2nd solvent): MeOH, linear gradient according to following table).

Gradient Table

| | Min | Solvents | % 2nd |
|---|---|---|---|
| 1 | 0.0 | AB | 0 |
| 2 | 9.5 | AB | 4 |
| 3 | 9.2 | AB | 14 |
| 4 | 1.4 | AB | 81 |

The product was obtained as white foam in a yield of 86%.

¹H NMR (400 MHz, DMSO-d6) δ 12.11 (s, 1H), 11.68 (s, 1H), 8.31 (s, 1H), 6.06 (d, 1H), 5.74 (dd, 1H), 5.47 (dd, 1H), 5.41 (t, 1H), 4.21 (q, 1H), 3.68 (m, 2H), 2.78 (m, 1H), 2.12 (s, 3H), 1.99 (s, 3H), 1.13 (s, 3H), 1.11 (s, 3H).

Synthesis of Compound 28 with the Following Structure:

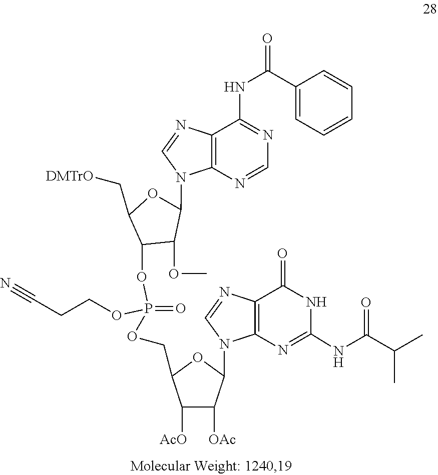

Compound 27 (1 eq.) and N6-Benzoyl-5′-O-DMT-2′-O-methyladenosine 3-CE phosphoramidite (1.1 eq.) were added to separate dry flasks and dried for 16 h at high vacuum. Flood flasks with argon and dissolve both educts in extra dry acetonitrile for DNA synthesis. Add dissolved 27 to the flask with dissolved N6-Benzoyl-5′-O-DMT-2′-O-methyladenosine 3-CE. Add Tetrazole (0.45M in acetonitrile, 2.5 eq.) and stir for 60 minutes. Oxidizer (0.1M Iodine in THE/Pyridine/water (77:21:2, v/v/v)) was added until the solution was red colored and stayed for 15 minutes without getting yellow again. A 1:1 mixture of aqueous sodium disulfide solution (5 wt. %) and citric acid solution (5 wt. %) was added and crude product was extracted with dichloromethane. Organic layer was washed with brine and dried with sodium sulfate. Solvent was evaporated and crude product was purified by flash column chromatography (Silica: 120 g, solvent A: Ethyl acetate, solvent B (2nd solvent): MeOH, linear gradient according to following table)

Gradient Table

| | Min | Solvents | % 2nd |
|---|---|---|---|
| 1 | 0.0 | AB | 0 |
| 2 | 33.5 | AB | 25 |
| 3 | 0.8 | AB | 80 |
| 4 | 3.7 | AB | 80 |

The product was obtained as white foam in a yield of 74%.

¹H-NMR (400 MHz, DMSO-d₆) δ 12.12 (s, 1H), 11.57 (s, 1H), 11.26 (s, 1H), 8.63 (d, 1H), 8.60 (d, 1H), 8.27 (d, 1H), 8.06 (d, 1H), 7.63 (t, 1H), 7.57 (t, 2H), 7.37 (t, 2H), 7.23 (m, 7H), 6.83 (m, 4H), 6.23 (d, 1H), 6.13 (t, 1H), 5.83 (m, 1H), 5.53 (m, 1H), 5.28 (m, 1H), 5.05 (m, 1H), 4.43 (m, 4H), 4.23 (m, 2H), 3.71 (d, 6H), 3.41-3.34 (m, 7H), 2.91 (m, 2H), 2.75 (m, 1H), 2.11 (d, 3H), 2.03 (d, 3H).

³¹P-NMR (162 MHz, DMSO-d6), δ −2.36 (d)

ESI-MS: [M−H]: 334.0557, Calculated 334.0558.

Synthesis of Compound 29 with the Following Structure:

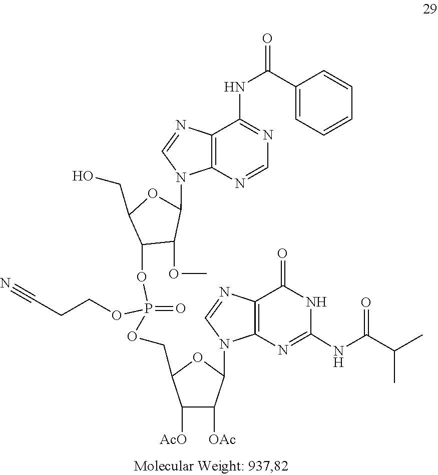 [SMILES: COC1C(OP(=O)(OCCC#N)OCC2OC(n3cnc4c(=O)[nH]c(NC(=O)C(C)C)nc43)C(OC(C)=O)C2OC(C)=O)C(CO)OC1n1cnc2c(NC(=O)c3ccccc3)ncnc21]

Compound 28 (1 eq.) was dissolved in aqueous acetic (80% v/v) and reaction mixture was stirred for 1 h. Solvent was evaporated and residual crude product coevaporated with methanol. Afterwards, product was purified by flash chromatography (Silica: 120 g, solvent A: Ethyl acetate, solvent B (2nd solvent): MeOH, linear gradient according to following table)

| | Min | Solvents | % 2nd |
|---|---|---|---|
| 1 | 0.0 | AB | 0 |
| 2 | 5.0 | AB | 15 |
| 3 | 20.0 | AB | 35 |
| 4 | 1.0 | AB | 80 |
| 5 | 3.0 | AB | 80 |

The product was obtained as white foam with a yield of 98%.

¹H NMR (400 MHz, DMSO-d₆) δ 12.13 (s, 1H), 11.59 (d, 1H), 11.26 (d, 1H), 8.77 (m, 2H), 8.29 (m, 1H), 8.05 (d, 2H), 7.65 (t, 1H), 7.55 (t, 1H), 6.20 (m, 1H), 6.14 (m, 1H), 5.85 (m, 1H), 5.55 (m, 1H), 5.40 (m, 1H), 5.21 (m, 1H), 4.83, (q, 1H), 4.48 (m, 3H), 4.33 (m, 1H), 4.28 (m, 2H), 3.64 (m, 2H), 3.42-3.33 (m, 5H), 2.98 (q, 2H), 2.76 (m, 1H), 2.15 (d, 3H), 2.03 (d, 3H), 1.11 (m, 6H).

³¹P-NMR (162 MHz, DMSO-d6), δ −2.38 (d)

Synthesis of Compound 30 with the Following Structure:

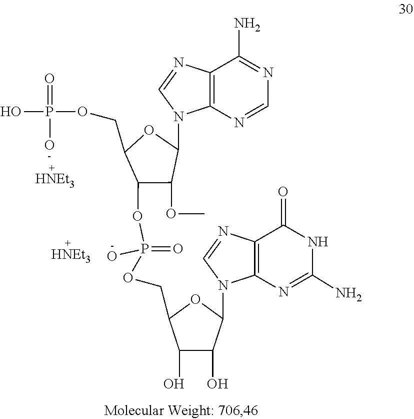 [SMILES: COC1C(OP(=O)(O)OCC2OC(n3cnc4c(=O)[nH]c(N)nc43)C(O)C2O)C(COP(=O)(O)O)OC1n1cnc2c(N)ncnc21]

Compound 29 (1 eq.) was dissolved in dry Acetonitrile under protective gas atmosphere. Bis(2-cyanoethyl)-N,N-diisopropylphosphoramidite (2 eq.) and Tetrazole (0.45M in dry acetonitrile, 2 eq.) was added and stirred for 30 minutes. Oxidizer (0.1 M Iodine in THE/Pyridine/water (77:21:2, v/v/v)) was added until red color was observed and stayed for 15 minutes. A 1:1 mixture of aqueous sodium disulfide solution (5 wt. %) and citric acid solution (5 wt. %) was added and crude product was extracted with dichloromethane. Solvent was evaporated and the residue was coevaporated with methanol. Protected crude product was dissolved in a 1:1 solution of methanol/concentrated ammonia and stirred for 48 h. Ammonia was evaporated and crude product was dissolved in water. The crude product was purified by ion exchange chromatography with Macro-Prep High-Q resin (BioRad) using solvent A: Water, solvent B (2nd solvent): 1 M TEAB with the following gradient:

| | Min | Solvents | % 2^{nd} |
|---|---|---|---|
| 1 | 0 | AB | 0 |
| 2 | 45 | AB | 100 |
| 3 | 0 | AB | 100 |
| 4 | 10 | AB | 100 |

Product was gained as white powder in a yield of 73%.

¹H NMR (400 MHz, DMSO-d₆) δ 8.48 (s, 1H), 8.15 (s, 1H), 7.96 (s, 1H), 7.32 (s, 2H), 6.69 (s, 2H), 6.02 (d, 1H), 5.70 (d, 1H), 4.88 (m, 1H), 4.58 (t, 1H), 4.54 (t, 1H), 4.32 (m, 1H), 4.22 (m, 1H), 4.02 (m, 1H), 3.96 (m, 2H), 3.90 (s, 2H), 3.35 (s, 3H).

³¹P-NMR (162 MHz, DMSO-d₆), δ −0.84 (s), −1.85 (s)

ESI-MS: [M−H]: 705.1190, Calculated 705.1189.

Synthesis of Compound 31 with the Following Structure:

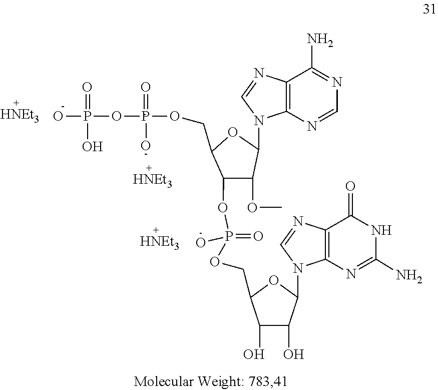 [SMILES: COC1C(OP(=O)(O)OCC2OC(n3cnc4c(=O)[nH]c(N)nc43)C(O)C2O)C(COP(=O)(O)OP(=O)(O)O)OC1n1cnc2c(N)ncnc21]

Compound 30 (1 eq.) was dissolved in dry dimethylsulfoxide. Cyanoethylphosphate-imidazolide (3 eq.) and magnesium chloride (10 eq.) were added and the reaction mixture was stirred for 24 h. DL-Dithiothreitol (3 eq.) and DBU (100 eq.) were added and the reaction was stirred for 5 h. DMSO was removed at reduced pressure. Crude product was dissolved in water and pH was adjusted to 8.0. Purification was carried out by ion exchange chromatography with Macro-Prep High-Q resin using the following gradient: Solvent A: Water, Solvent B (2^{nd} Solvent): 1M TEAB

| Step | Min | Solvents | % 2^{nd} Solvent |
|---|---|---|---|
| 1 | 0 | AB | 0 |
| 2 | 45 | AB | 100 |
| 3 | 20 | AB | 100 |

The solvent of product fractions was evaporated yielding in 64% of a white powder.

¹H NMR (400 MHz, DMSO-d₆) δ 8.50 (s, 1H), 8.15 (s, 1H), 7.96 (s, 1H), 7.31 (s, 2H), 6.76 (s, 2H), 6.00 (d, 1H), 5.69 (d, 1H), 4.91 (m, 1H), 4.63 (m, 2H), 4.29 (m, 1H), 4.24 (m, 1H), 4.01 (m, 4H), 3.87 (m, 1H), 3.49 (m, 2H), 3.31 (s, 3H).

³¹P-NMR (162 MHz, DMSO-d₆), δ 1.95 (s), −10.70 (d), −11.50 (d)

ESI-MS: [M+H]: 787.098, Calculated 787.099.

Synthesis of Compound 32 with the Following Structure:

The synthesis of 32 starts from m7Guanin-9-propyl-monophosphatethat was previously synthesized according to route 1, involving 1,3-propanediol as starting material.

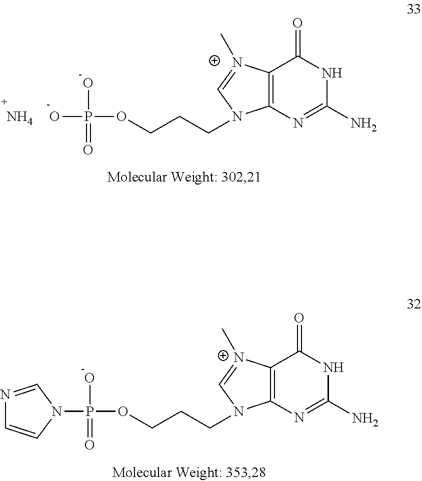

Compound 33 (1 eq.) was dissolved in DMSO and carbonyldiimidazole (20 eq.) was added in two portions over 24 h. Reaction mixture was stirred for 48-72 h and quenched by addition of water (20 eq.). The crude product was precipitated by pouring on ice cold sodium perchlorate solution in acetone (12 wt. %). The precipitate was sedimented by centrifugation and washed with ice cold acetone, yielding in 67% of a greyish solid.

ESI-MS: [M+H]: 354.107, Calculated 354.107.

### Synthesis of Compound 25:

Compound 32 (1 eq.) was dissolved in water/acetonitrile (1:1, v/v) or in pure water. The diphosphorylated AmG-dinucleotide 31 (1.0 eq.) was added. After dissolution of the educts, magnesium chloride or zinc chloride (10 eq.) was added and the reaction mixture was stirred for 16-24 h. The reaction was quenched by addition of EDTA (11 eq.) and adjusted to pH 7.0 with aqueous ammonia solution. The reaction mixture was desalted using RP-HPLC using solvent A: 5 mM ammonium acetate, solvent B: 90% Methanol in water with the gradient program in Table Ex-15.

TABLE Ex-15

| Time [min] | A [%] | B [%] |
|---|---|---|
| 0 | 100 | 0 |
| 10 | 100 | 0 |
| 20 | 85 | 15 |
| 21 | 5 | 95 |
| 31 | 5 | 95 |
| 32 | 100 | 0 |
| 48 | 100 | 0 |

The desalted reaction mixture was purified by RP-HPLC using solvent A: 100 mM triethylammonium acetate (pH7), solvent B: 80% acetonitrile in water. The gradient program is described in table Ex-16.

TABLE Ex-16

| Time [min] | A [%] | B [%] |
|---|---|---|
| 0 | 100 | 0 |
| 40 | 60 | 40 |
| 41 | 100 | 0 |
| 51 | 100 | 0 |

The product was obtained as white solid in a yield of 18% and dissolved in ultrapure water resulting in a 100 mM aolution.

¹H NMR (400 MHz, DMSO-d₆) δ 8.82 (s, 1H), 8.35 (s, 1H), 8.09 (s, 1H), 7.94 (s, 1H), 6.00 (d, 1H), 5.80 (d, 1H), 4.94 (m, 1H), 4.71 (m, 1H), 4.49 (m, 2H), 4.39 (m, 1H), 4.34 (m, 3H), 4.27 (m, 2H), 4.21 (m, 3H), 4.15 (m, 2H), 3.99 (q, 2H) 3.98 (s, 3H), 3.46 (s, 3H), 2.05 (m, 2H),

³¹P-NMR (162 MHz, DMSO-d₆), δ −0.84 (s), −11.13 (d), −11.48 (d), −22.73 (t)

ESI-MS: [M−H]: 1070.147, Calculated: 1070.147.

Similar compounds can be synthesized in accordance with the above synthesis route when starting from other educt compounds.

### Example 2: mRNA Preparation

Initially, mRNAs with two different caps were prepared as outlined in the present example, namely i) mRNA with the commercially available m7G(5′)ppp(5′)G Cap Analogue (from Thermo Fisher Scientific, referred to herein as “mCap”, see also the structure below) and ii) mRNA with the acyclovir cap of the present invention (compound 19, see example 1). mRNA i) is referred to below as “mCap mRNA” and mRNA ii) is referred to below as “acyclovir cap mRNA”. The structure of mCap is as follows:

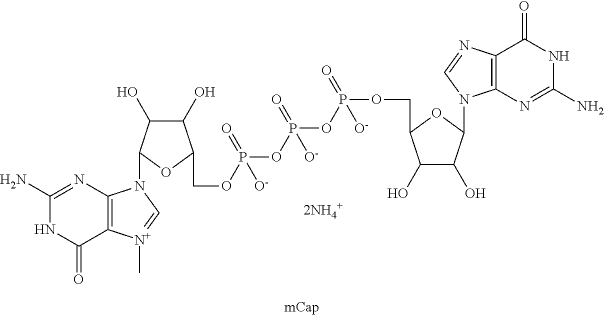 [SMILES: C[n+]1cn(C2OC(COP(=O)([O-])OP(=O)([O-])OP(=O)([O-])OCC3OC(n4cnc5c(=O)[nH]c(N)nc54)C(O)C3O)C(O)C2O)c2nc(N)[nH]c(=O)c21]

Furthermore, mRNAs with the following caps were prepared (see FIGS. 1 and 3): acyclovir, propylene (corresponding to propylene linked cap0), ethylene, diethyleneglycol, butylene, phosphonate variant 2, phosphonate variant 4, phosphonate variant 5, phosphonate variant 6, phosphonate variant 9, phosphonate variant 10, phosphonate variant 12, and Compound 25 (corresponding to propylene linked cap1).

A DNA sequence was introduced into a modified pUC19 derived vector backbone to comprise a 5′-UTR with a ribozyme cleavage site (the HSD17B4 5′ UTR), a 3′-UTR, a histone-stem-loop structure and a stretch of adenine nucleotides (A100), at the 3′-terminal end. The obtained plasmid DNA was transformed and propagated in bacteria using common protocols and plasmid DNA was extracted, purified, and enzymatically linearized using a restriction enzyme. The obtained linearized plasmid DNA was used for RNA in vitro transcription as outlined next to obtain the mRNA with the sequence shown in SEQ ID NO: 1.

Linearized plasmid DNA template (50 μg/ml) was transcribed at 37° C. for 3-5 hours in 80 mM HEPES/KOH, pH 7.5, 24 mM MgCl₂, 2 mM spermidine, 40 mM DTT, 5 U/ml pyrophosphatase (Thermo Fisher Scientific), 200 U/ml RiboLock RNase inhibitor (Thermo Fisher Scientific), 5000 U/ml T7 RNA polymerase (Thermo Fisher Scientific).

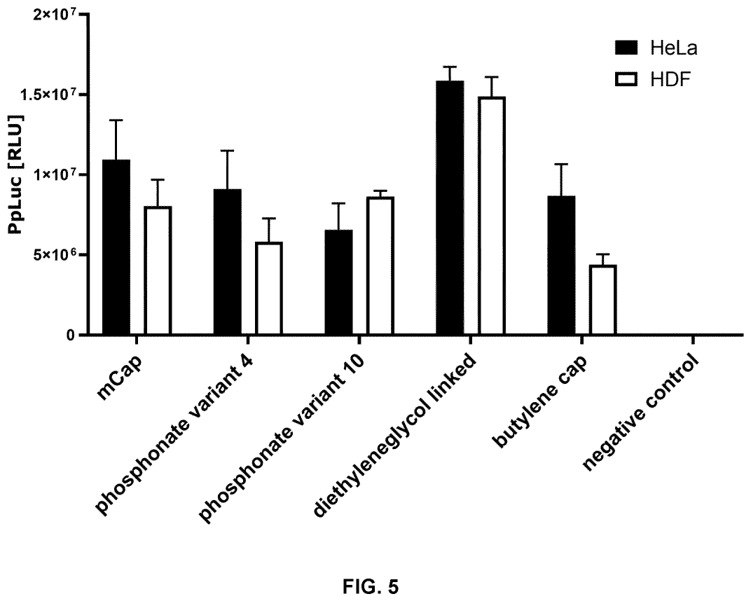

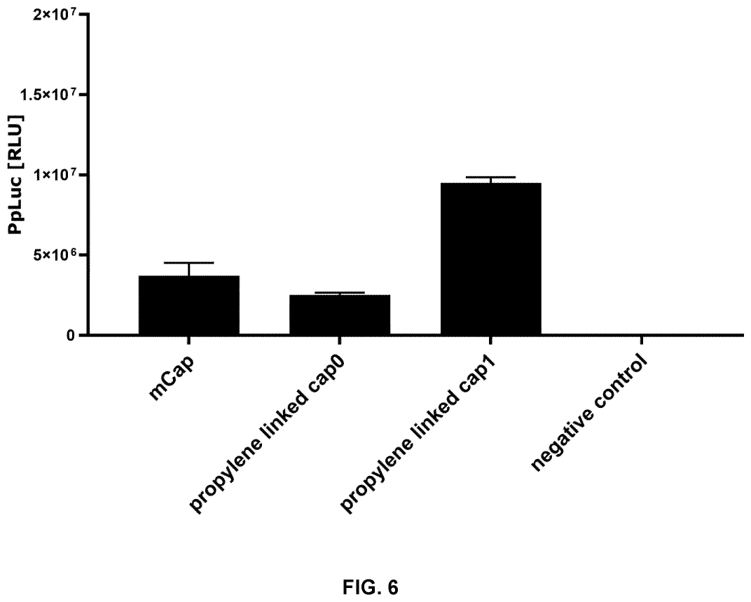

The non-modified and modified nucleotide mixture was sequence-optimized (herein referred to as sequence-optimized IVT-mix) preferably in accordance with a procedure as described in WO2015/188933. Example 1. In short, the sequence-optimized IVT-mix comprised the four ribonucleoside triphosphates (NTPs) GTP, ATP, CTP and (modified) UTP in a sequence optimized ratio, wherein the fraction of each of the four ribonucleoside triphosphates in the sequence-optimized IVT-mix corresponded to the fraction of the respective nucleotide in the mRNA molecule to be synthetized, a buffer, a DNA template, and an RNA polymerase. Accordingly, for mRNAs i) and ii) as set out above, the concentrations of the nucleotides were 0.42 mM ATP, 0.57 mM CTP, 0.28 mM UTP, and 0.5 mM GTP (all Thermo Fisher Scientific). Transcription was carried out in the presence of 2.0 mM mCap in order to obtain mRNA i), and in the presence of 2.0 mM acyclovir cap in order to obtain mRNA ii). mRNAs used in example 3, Table Ex-8, example 4, Table Ex-9 and example 5, FIG. 5 were transcribed with the following nucleotide concentrations: 3.18 mM ATP, 4.33 mM CTP, 2.13 mM N1-Methyl-pseudouridine and 15.23 mM of the respective cap0 analog. mRNAs used in example 5, FIG. 6 were transcribed with the following nucleotide concentrations: 3.19 mM ATP, 4.33 mM CTP, 2.13 mM UTP, 3.80 mM GTP and 5.0 mM cap1 analog (compound 25).

Following RNA in vitro transcription, linear DNA templates were removed by Pulmozyme (Ratiopharm) (2500 U/ml, 3.2 mM CaCl₂, 30 min at 37° C.). The obtained mRNAs i) and ii) as well as the mRNAs used in example 3, Table Ex-6; example 4, Table Ex-7; and example 5, FIG. 5 were purified using RP-HPLC (PureMessenger®; according to WO2008/077592). The obtained mRNAs used in example 3, Table Ex-8; example 4, Table Ex-9; and example 5, FIG. 6, were purified using Monarch RNA cleanup kit or Qiagen RNeasy mini kit according to the protocol of the manufacturer and used for dsRNA determination, capping analysis and in vitro expression experiments (example 5).

### Example 3: Determination of the Capping Efficacy when Using Different Cap Analoga

The capping efficacy can be analyzed by an HPLC assay of a 5′ fragment of mRNAs obtained according to the protocol of example 2. Thus, the peaks obtained in such an HPLC assay are indicative of i) correctly capped mRNA, ii) capped mRNA lacking a single nucleotide G (which is a typical side product if T7 RNA-polymerase is used), iii) uncapped mRNA and iv) further products of unknown structure. 5′ fragments for this kind of analysis are typically 15 to 20 nucleotides in length.

In order to provide fragments of a length of 18 nucleotides (assuming that the Cap-nucleotide is present, the fragment is 18 nucleotides long; for uncapped mRNAs, the fragment is accordingly 17 nucleotides long) for HPLC analysis, the mRNAs i) and ii) obtained in example 2 were first cleaved at the above-mentioned 5′ ribozyme cleavage site using a ribozyme designed to cleave at the relevant position. The ribozyme reaction contained 150 μM of the respective mRNA, 150 μM of the ribozyme, 50 mM NaCl and 0.625 mM EDTA in a total reaction volume of 120 μL. For the annealing reaction, the mixture was incubated in a PCR cycler for 3 min at 95° C., followed by a cool down ramp of 0.1° C. per second to a final temperature of 25° C., with a final incubation of 10 min at 25° C. After addition of 30 μL of a 200 mM MgCl₂ solution buffered with 250 mM TRIS/HCl (pH 7.5), the reaction mix was incubated at 25° C. for 1 h. The reaction was stopped by addition of 24 μL of a 250 mM EDTA solution. RNA-only and Ribozyme-only controls were prepared per RNA and ribozyme, respectively.

Prior to HPLC analysis, 1446 μL HPLC-grade water and 180 μL 1 M TEAA solution were added to the stopped reaction mix and mixed vigorously. HPLC analysis was performed using a AQUITY PREMIER Oligonucleotide C18 130 Å column (2.1×50 mm, 1.7 μm particle size, Waters) with a column temperature of 65° C. and a flowrate of 0.65 mL/min. Eluent A consisted of 0.1 M TEAA in HPLC grade water, pH 7.0. Eluent B consisted of 0.1 m TEAA, 15% ACN (v/v) in HPLC grade water, pH 7.0. A specific gradient was applied to separate the short RNA fragments (see Table Ex-5). RNA peaks were detected by a UV/VIS spectrophotometer at 260 nm. Peak areas were integrated resulting in the relative fractions of differently capped mRNA.

TABLE Ex-5
HPLC gradient for capping analysis

| Time (min) | Fraction Eluent B (%) |
|---|---|
| 0-3 | 14 |
| 3-5 | 14-19 |
| 5-14 | 19-21 |
| 14-15 | 21 |
| 15-20 | 21-100 |
| 20-23.5 | 100 |
| 23.5-25 | 100-14 |

The identities of the peaks and the obtained relative peak areas are shown in Table Ex-6 and Ex-8.

TABLE Ex-6
peak identities and relative peak areas

| | Relative peak area [%] | | | |
|---|---|---|---|---|
| | Correctly | Capped RNA | Uncapped | Unidentified |
| mRNA-sample | capped RNA | minus G | RNA | peaks |
| mCap | 61.5 | 8.6 | 19.8 | 10.1 |
| Acyclovir cap | 69.9 | 6.3 | 20.0 | 3.8 |

TABLE Ex-8
peak identities and relative peak areas

| | Relative peak area [%] | | | |
|---|---|---|---|---|
| | Correctly | Capped | | |
| mRNA- | capped | RNA | Uncapped | Unidentified |
| sample | RNA | minus G | RNA | peaks |
| Acyclovir cap | 67.3 | 15.1 | 16.6 | 1.0 |
| Propylene Cap | 64.7 | 21.6 | 12.3 | 1.4 |
| Phosphonate | 76.9 | 6.3 | 15.0 | 1.8 |
| Variant 2 | | | | |
| Phosphonate | 75.5 | 4.2 | 14.9 | 5.4 |
| Variant 6 | | | | |
| Phosphonate | 77.1 | 4.3 | 13.1 | 5.5 |
| Variant 5 | | | | |
| Phosphonate | 80.5 | 16.6 | 2.6 | 0.3 |
| Variant 4 | | | | |
| Phosphonate | 73.4 | 4.4 | 15.7 | 6.5 |
| Variant 9 | | | | |
| Phosphonate | 75.5 | 8.6 | 12.2 | 3.7 |
| Variant 12 | | | | |
| Phosphonate | 63.0 | 23.4 | 8.7 | 4.9 |
| Variant 10 | | | | |
| Ethylene Cap | 67.3 | 15.4 | 14.5 | 2.8 |
| mCap | 63.3 | 12.2 | 20.3 | 4.2 |

It is evident from the above results shown in Table Ex-6 that the use of the acyclovir cap analog resulted in i) more correctly capped mRNA, ii) less incorrectly capped RNA (lacking a G), and iii) less incorrectly capped, unidentified structures compared to the mCap analog. The fraction of uncapped mRNA was more or less identical in both mRNA samples. It must be emphasized that it is not possible to analyze in the present assay how big the fraction of reversed cap-structures in the mCap mRNA sample was (i.e. the fraction of the incorrect reverse orientation G(5′)ppp(5′)m7GNNNNN . . . ). However, it can be assumed that a quite substantial fraction of at least about ⅓ of the “correctly capped RNA” relates to such reverse structures in the mCap mRNA sample. Such a reverse orientation is not possible when using the acyclovir cap analog because of the lacking OH-group at the required position such that all of the “correctly capped RNA” molecules in fact have the correct orientation in the acyclovir mRNA sample.

The results shown in Table Ex-8 are also positive with respect to the new cap-structures. It is noted that mRNA-samples in Table Ex-8 were purified differently than the mRNA samples shown in Table Ex-6.

### Example 4: Determination of the Presence of dsRNA

dsRNA can be an undesired side product of in vitro transcription and is mainly resulting from i) a 3′-extension of the run-off products annealing to complementary sequences in the body of the run-off transcript in cis (by folding back on the same RNA) or trans (by annealing to a second RNA) to form extended duplexes or to ii) hybridization of an antisense RNA molecule to the run-off transcript. The amount of dsRNA in an RNA preparation can be analyzed inter alia with an ELISA assay using antibodies specific for dsRNA, as described in the following.

9D5 antibody (specific for dsRNA, from absolute antibody) was diluted to 2 μg/ml in PBS and used to coat Nunc MaxiSorp® flat bottom 96-well plates (Thermo Fischer) with 100 μl for 2 h at room temperature. After coating, wells were washed three times using PBS-T (PBS and 0.05% Tween-20). Samples and standards were diluted in 1×TE buffer (AppliChem) and 100 μl were added to each well and incubated over night at 4° C. (approx. 20 h). After incubation, wells were washed three times using PBS-T. K2 antibody (Scicon) was diluted 1:200 in PBST and 100 μl were added to each well and incubated for 2 h at room temperature. Wells were washed three times using PBS-T. Anti-mouse IgM-HRP (Invitrogen) was diluted 1:50 in PBST and 100 μl were added to each well and incubated for 1 h at room temperature. Wells were washed three times using PBS-T. Color reagents A and B (R&D systems) were mixed in equal amounts and 100 μl were added to each well and incubated for 9 minutes. Plates were measured in a plate reader at OD450 and OD540. OD540 values were subtracted from OD450 values and used for the determination of absolute amounts of dsRNA with a lower limit for quantification of 0.03 ng of dsRNA per μg RNA. The results are given in Table Ex-7 and Ex-9.

TABLE Ex-7

| | RNA concentration | Blanked Data | dsRNA |
|---|---|---|---|
| mRNA-sample | during test (ng/μL) | (OD450-OD540) | (ng/μg RNA) |
| mCap | 100 | 1.32 | 0.09 |
| Acyclovir cap | 100 | 0.56 | 0.04 |

TABLE Ex-9

| | RNA | | |
|---|---|---|---|
| | concentration | | |
| | during test | Blanked Data | dsRNA |
| mRNA-sample | (ng/μL) | (OD450-OD540) | (ng/μg RNA) |
| Acyclovir cap | 1 | | 8.03 |
| Propylene Cap | 1 | | 11.19 |
| Phosphonate Variant 2 | 1 | | 7.433 |
| Phosphonate Variant 6 | 1 | | 4.52 |
| Phosphonate Variant 5 | 1 | | 9.58 |
| Phosphonate Variant 4 | 1 | | 7.08 |
| Phosphonate Variant 9 | 1 | | 8.94 |
| Phosphonate Variant 12 | 1 | | 6.17 |
| Phosphonate Variant 10 | 1 | | 8.10 |
| Ethylene Cap | 1 | | 8.51 |
| mCap | 1 | | 10.46 |

It is evident from the above results that the amount of undesired dsRNA in the preparation was reduced to more than half when the acyclovir cap analog was used compared to the mCap analog (see the results of Table Ex-7). The results shown in Table Ex-9 are also positive for the cap structures of the present application compared to the mCap.

### Example 5: Luciferase Expression Using mRNAs with Various Cap Analoga

Cells were seeded on 96 well plates (Sarstedt). HDF (human dermal fibroblast) and HeLa were seeded 24 hours before transfection in a compatible complete cell medium (10,000 cells in 200 μl/well). Cells were maintained at 37° C., 5% CO₂. The day of transfection, the complete medium on cells was replaced with serum-free Opti-MEM medium (Gibco). Each RNA was complexed with Lipofectamine2000 at a ratio of 1/1.5 (w/v) for 20 minutes in Opti-MEM. Lipocomplexed mRNAs were then added to cells for transfection with 50 ng of RNA per well in a total volume of 200 μl. 90 minutes post start of transfection, complete supernatant (200 μl/well) of transfection solution was exchanged for 200 μl/well of complete medium. Cells were further maintained at 37° C., 5% CO₂ before harvesting. 24 hours post start of transfection cells were lysed to measure luciferase expression within cells. First, 100 μl of 1× passive lysis buffer (Promocell) was added to each well. Cells were shaken for 15 minutes at room temperature until there were incubated at −80° C. for at least one hour. After thawing, 20 μl of lysates were used to detect and measure luciferase activity via chemi-luminescence using ATP and D-Luziferin in a Beetlejuice buffer system (p.j.k.). To this end, plates were introduced into a in a plate reader (Tristar 2S Berthold) with injection device for Beetle-juice containing substrate for firefly luciferase. Per well, 50 μl of beetle-juice were added. Raw data containing relative light units were used to plot differences between mRNAs derived from cap analogs.

Expression Analysis of Cap0 mRNA:

All tested cap0 mRNAs (cap analogs: acyclovir linked, propylene linked, phosphonate variant 1, phosphonate variant 2, phosphonate variant 3, phosphonate variant 4, phosphonate variant 5, phosphonate variant 6, phosphonate variant 9, phosphonate variant 10, phosphonate variant 11, phosphonate variant 12, ethylene linked, diethyleneglycol linked and butylene cap) showed expression of PpLuc protein after transfection of 50 ng mRNA in HDF and HeLa cells.

FIG. 5 shows the PpLuc expression of the phosphonate variant 4, phosphonate variant 10, diethyleneglycol linked and the butylene capped mRNA compared to mCap mRNA and an untransfected negative control. The results obtained with Cap0 analogs demonstrate that all tested analogs are functional and are able to initiate protein expression. Moreover, certain Cap0 analogs have a comparable translation efficiency or even an outperforming translation efficiency compared to mCap. Accordingly, these Cap0 structures are particularly suitable for the development of Cap1 analogs.

Expression Analysis of Cap1 mRNA:

The tested cap1 mRNA with the propylene linked cap analog (Compound 25) showed significant higher PpLuc protein expression compared to mCap or the corresponding cap0 dinucleotide after transfection of 50 ng mRNA in HDF cells (see FIG. 6). The results obtained with Cap1 analogs demonstrate that mRNA that has been capped using a Cap1 analog of the present invention shows a more than 2.5 fold higher translation efficiency compared to an mRNA that has been capped using mCap.

1. A compound of formula (I): 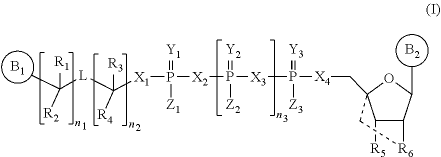
or a salt, stereoisomer, tautomer, or deuterated version thereof, wherein
R₅ is 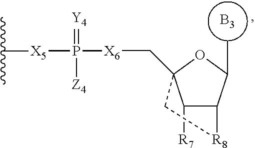
R₇ is OH or 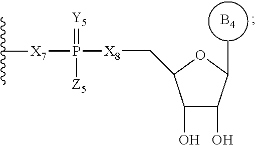
ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
n₃ is selected from 0, 1 or 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
each of X₁ through X₈ is independently O, S, NH or CH₂;
each of Y₁ through Y₅ is independently O, S or Se;
each of Z₁ through Z₅ is independently OH, SH or BH_{3;}
R₆ is (i) selected from the group consisting of H, OH, OC₁-C₃-alkyl, and Opropargyl, wherein the dashed methylene bridge between R₆ and the 4′ C is absent, or (ii) O, wherein the dashed methylene bridge between R₆ being O and the 4′ C is present;
R₈ is (i) selected from the group consisting of H, OH, OC₁-C₃-alkyl, and Opropargyl, wherein the dashed methylene bridge between R₃ and the 4′ C is absent, or (ii) O, wherein the dashed methylene bridge between R₃ being O and the 4′ C is present; and
each of ring B₂ through ring B₄ is independently a nucleobase, a modified nucleobase or a nucleobase analog.

2. The compound according to claim 1, wherein
n₃ is 1;
R₆ is selected from the group consisting of H, OH, OC₁-C₃-alkyl, and Opropargyl, wherein the dashed methylene bridge between R₆ and the 4′ C is absent; and
R₈ is selected from the group consisting of H, OH, OC₁-C₃-alkyl, and Opropargyl, wherein the dashed methylene bridge between R₈ and the 4′ C is absent.

3. The compound according to claim 1 or 2, wherein B₂ is selected from the group consisting of guanine, a modified guanine, a guanine analog, adenine, a modified adenine, and an adenine analog.

4. The compound according to any one of the preceding claims, wherein B₃ is guanine, a modified guanine or a guanine analog.

5. The compound according to any one of the preceding claims, wherein each of R₁ through R₄ is independently H, OH, SH, NH₂ or halogen.

6. The compound according to any one of claims 1 to 5, wherein R₅ is 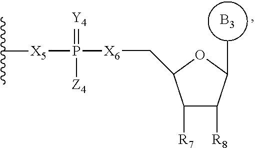
wherein R₇ and R₈ are each OH.

7. The compound according to claim 6, wherein R₆ is H or OC₁-C₃-alkyl, preferably wherein R₆ is OCH₃.

8. The compound according to any one of claims 1 to 5, wherein R₅ is 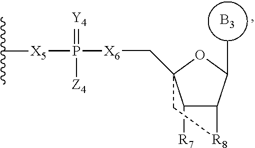
wherein R₇ is

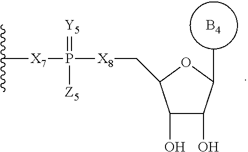

9. The compound according to claim 8, wherein
R₆ is H or OC₁-C₃-alkyl, wherein the dashed methylene bridge between R₆ and the 4′ C is absent, preferably wherein R₆ is OCH₃; and/or
R₈ is H or OC₁-C₃-alkyl, wherein the dashed methylene bridge between R₈ and the 4′ C is absent, preferably wherein R₈ is OCH₃.

10. The compound according to any one of the preceding claims, wherein ring B₁ is a modified guanine.

11. The compound according to any one of the preceding claims, wherein ring B₁ is N⁷-methylguanine.

12. The compound according to any one of the preceding claims, wherein X₁ is CH₂ or O, and each of X₂ through X₈ is O.

13. The compound according to any one of the preceding claims, wherein each of Y₁ through Y₅ is O.

14. The compound according to any one of the preceding claims, wherein each of Z₁ through Z₅ is OH.

15. The compound according to any one of the preceding claims, wherein each of R₁ through R₄ is independently H or OH; or one of R₁ through R₄ is selected from the group consisting of CH₂, CH(OH), and C(OH)₂, and each of the remaining three of R₁ through R₄ is independently H or OH.

16. The compound according to any one of the preceding claims, wherein each of R₁ through R₃ is H and R₄ is H or OH.

17. The compound according to any one of the preceding claims, wherein each of R₁ through R₄ is H; or one of R₁ through R₄ is selected from the group consisting of CH₂, CH(OH), and C(OH)₂, and each of the remaining three of R₁ through R₄ is H or OH.

18. The compound according to any one of the preceding claims, wherein n₁ and n₂ are each independently selected from an integer ranging from 0 to 3.

19. The compound according to any one of the preceding claims, wherein n₁ is selected from 0, 1, 2 or 3; and n₂ is selected from 0, 1 or 2.

20. The compound according to claim 19, wherein n₁ is 0; and n₂ is selected from 1 or 2.

21. The compound according to claim 19, wherein n₁ is 1; and n₂ is selected from 1 or 2.

22. The compound according to claim 19, wherein n₁ is 2; and n₂ is selected from 1 or 2.

23. The compound according to claim 19, wherein n₁ is selected from 1 or 2; and n₂ is 0.

24. The compound according to claim 19, wherein n₁ is selected from 1 or 2; and n₂ is 1.

25. The compound according to claim 19, wherein n₁ is selected from 1 or 2; and n₂ is 2.

26. The compound according to claim 19, wherein n₁ is 3; and n₂ is 1.

27. The compound according to claim 19, wherein n₁ is 2; and n₂ is 0.

28. The compound according to any one of the preceding claims, wherein L is selected from the group consisting of CH₂, O, S, SO, SO₂ and CH(OH).

29. The compound according to any one of claims 1 to 16, wherein (i) each of R₁ through R₄ is H; (ii) n₁ is selected from 0, 1 or 2; (iii) n₂ is selected from 1 or 2; (iv) L is selected from CH₂ and O; and (v) X₁ is 0.

30. The compound according to any one of claims 1 to 16, wherein (i) each of R₁ through R₃ is H; (ii) R₄ is H or OH; (iii) wherein each of n₁ and n₂ is selected from 1 or 2; (iv) L is selected from CH₂, O and CH(OH); and (v) X₁ is O.

31. The compound according to any one of claims 1 to 16, wherein (i) each of R₁ through R₄ is H; (ii) n₁ is selected from 1, 2 or 3; (iii) n₂ is selected from 0, 1 or 2; (iii) L is selected from S, SO and SO₂; and (iv) X₁ is CH₂.

32. A cap analog comprising a 5′ terminal acyclonucleoside, wherein the acyclonucleoside comprises a linear unbranched structure or a linear single-branched structure instead of a ribose, wherein the cap analog is a cap1 analog or a cap2 analog and wherein the 5′ terminal acyclonucleoside is optionally deuterated.

33. The cap analog according to claim 32, wherein the linear unbranched structure has the structure of formula (II): 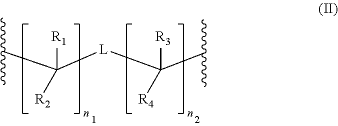
wherein
each of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).

34. The cap analog according to claim 33, wherein each of R₁ through R₄ is independently H or OH; n₁ and n₂ are each independently selected from an integer ranging from 0 to 3; and L is selected from the group consisting of CH₂, O, S, SO, SO₂ and CH(OH).

35. The cap analog according to claim 32, wherein the linear single-branched structure has the structure of formula (II): 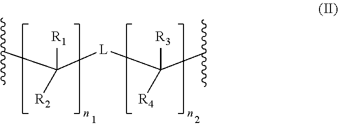
wherein
one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).

36. The cap analog according to claim 35, wherein one of R₁ through R₄ is selected from the group consisting of CH₃, CH₂(OH), and CH(OH)₂, and each of the remaining three of R₁ through R₄ is independently H or OH; n₁ and n₂ are each independently selected from an integer ranging from 0 to 3; and L is selected from the group consisting of CH₂, O, S, SO, SO₂ and CH(OH).

37. The cap analog according to any one of claims 32 to 36, wherein the acyclonucleoside comprises as the nucleobase guanine, a modified guanine or a guanine analog.

38. An RNA molecule comprising at least three nucleotides and comprising a 5′ end of formula (III): 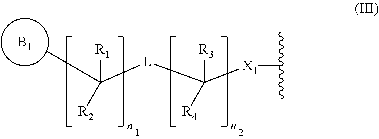
wherein
ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
X₁ is O, S, NH or CH₂; and
wherein the 5′ end is optionally deuterated.

39. The RNA molecule according to claim 38, wherein each of R₁ through R₄ is independently H or OH; n₁ and n₂ are each independently selected from an integer ranging from 0 to 3; L is selected from the group consisting of CH₂, O, S, SO, SO₂ and CH(OH); and X₁ is 0 or CH₂.

40. The RNA molecule according to claim 38 or 39, wherein ring B₁ is a modified guanine, preferably N⁷-methylguanine.

41. The RNA molecule according to any one of claims 38 to 40, wherein the RNA molecule comprises a 5′ end of formula (I): 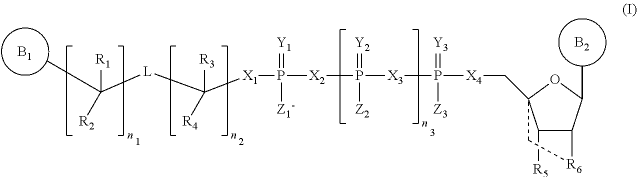
wherein
R₅ is 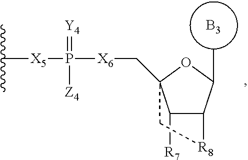
wherein
R₇ is 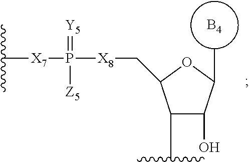
and wherein
n₃ is selected from 0, 1 or 2;
each of X₂ through X₈ is independently O, S, NH or CH₂;
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) O, wherein the dashed methylene bridge between R₆ being O and the 4′ C is present;
R₈ is (i) selected from the group consisting of H, OH, OC₁-C₃-alkyl, and Opropargyl, wherein the dashed methylene bridge between R₈ and the 4′ C is absent, or (ii) O, wherein the dashed methylene bridge between R₈ being O and the 4′ C is present; and
each of ring B₂ through ring B₄ is independently a nucleobase, a modified nucleobase, or a nucleobase analog.

42. The RNA molecule according to claim 41, wherein R₆ is OC₁-C₃-alkyl, preferably wherein R₆ is OCH₃, wherein the dashed methylene bridge between R₆ and the 4′ C is absent.

43. The RNA molecule according to claim 41 and 42, wherein R₈ is OC₁-C₃-alkyl, preferably wherein R₈ is OCH₃, wherein the dashed methylene bridge between R₈ and the 4′ C is absent.

44. The RNA molecule according to claim 41, wherein R₆ is OC₁-C₃-alkyl, preferably wherein R₆ is OCH₃, wherein the dashed methylene bridge between R₆ and the 4′ C is absent; and R₈ is OC₁-C₃-alkyl, preferably wherein R₈ is OCH₃, wherein the dashed methylene bridge between R₈ and the 4′ C is absent.

45. The RNA molecular according to any one of claims 41 to 43, wherein
n₃ is 1;
each of X₂ through X₈ is O;
each of Y₁ through Y₅ is O;
each of Z₁ through Z₅ is OH; and
each of ring B₂ through ring B₄ is a nucleobase.

46. An RNA molecule comprising at least three nucleotides and comprising a 5′ terminal acyclonucleoside, wherein the acyclonucleoside comprises a linear unbranched structure or a linear single-branched structure instead of a ribose, and wherein the 5′ terminal acyclonucleoside is optionally deuterated.

47. The RNA molecule according to claim 46, wherein the linear unbranched structure has the structure of formula (II): 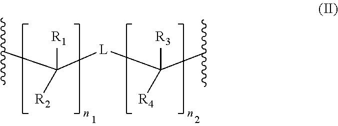
wherein
each of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).

48. The RNA molecule according to claim 47, wherein each of R₁ through R₄ is independently H or OH; n₁ and n₂ are each independently selected from an integer ranging from 0 to 3; and L is selected from the group consisting of CH₂, O, S, SO, SO₂ and CH(OH).

49. The RNA molecule according to claim 46, wherein the linear single-branched structure has the structure of formula (II): 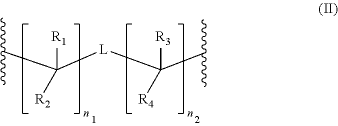
wherein
one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10; and
L is selected from the group consisting of CH₂, O, S, SO, SO₂, N, CH(OH), CH(SH), and CH(halogen) with the proviso that, if (i) n₁ is 0 and/or (ii) n₂ is 0, L is selected from the group consisting of CH₂, CH(OH), CH(SH) and CH(halogen).

50. The RNA molecule according to claim 49, wherein one of R₁ through R₄ is selected from the group consisting of CH₃, CH₂(OH), and CH(OH)₂, and each of the remaining three of R₁ through R₄ is independently H or OH; n₁ and n₂ are each independently selected from an integer ranging from 0 to 3;
and L is selected from the group consisting of CH₂, O, S, SO, SO₂ and CH(OH).

51. The RNA molecule according to any one of claims 46 to 50, wherein the acyclonucleoside comprises as the nucleobase guanine, a modified guanine or a guanine analog.

52. An RNA molecule whose 5′ end comprises a compound according to any one of claims 1 to 31.

53. An in vitro method for synthesizing an RNA molecule, the method comprising reacting nucleotides, (i) the compound according to any one of claims 1 to 31 or (ii) the cap analog according to any one of claims 32 to 37, and a DNA template in the presence of a DNA-dependent RNA polymerase under conditions suitable for the transcription of the DNA template into an RNA molecule by the DNA-dependent RNA polymerase.

54. An RNA molecule obtained by the in vitro method according to claim 53.

55. The RNA molecule according to any one of claims 38 to 52 and 54, wherein the RNA molecule comprises at least one chemical modification.

56. The RNA molecule according to claim 55, wherein the at least one chemical modification is selected from the group consisting of a base modification, a sugar modification and a backbone modification.

57. The RNA molecule according to claim 55 or 56, wherein the at least one chemical modification is a base modification, wherein the base modification is preferably selected from the group consisting of pseudouridine (psi or ψ), N1-methylpseudouracil (N1Mpsi or N1Mψ), 1-ethylpseudouracil, 2-thiouracil (s2U), 4-thiouracil, 5-methylcytosine, 5-methyluracil, 5-methoxyuracil, and any combination thereof.

58. The RNA molecule according to any one of claims 38 to 52 and 54 to 57, wherein the RNA molecule is a coding RNA comprising at least one coding sequence, preferably wherein the coding RNA is an mRNA.

59. The RNA molecule according to any one of claims 38 to 52 and 54 to 58, wherein the RNA molecule is a therapeutic mRNA.

60. A composition comprising the RNA molecule according to any one of claims 38 to 52 and 54 to 59.

61. The composition according to claim 60, wherein the composition is a pharmaceutical composition.

62. A kit comprising (i) the compound according to any one of claims 10 to 31 or (ii) the cap analog according to any one of claims 32 to 37, and a DNA-dependent RNA polymerase.

63. The kit according to claim 62, wherein the kit further comprises nucleotides.

64. The kit according to claim 62 or 63, wherein the kit further comprises a ribonuclease inhibitor.

65. The kit according to any one of claims 62 to 64, wherein the kit further comprises a buffer.

66. Use of (i) the compound according to any one of claims 1 to 31 or (ii) the cap analog according to any one of claims 32 to 37 in an in vitro transcription reaction for producing a capped RNA molecule.

67. The use according to claim 66, wherein the capped RNA molecule is the RNA molecule according to any one of claims 38 to 52 and 54 to 59.

68. A process for preparing a compound of formula (I): 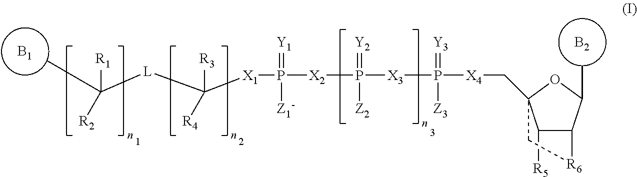
or a salt, stereoisomer, tautomer, or deuterated version thereof, wherein
R₅ is 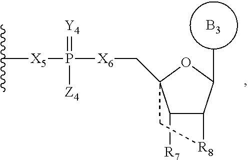
wherein
R₇ is OH or 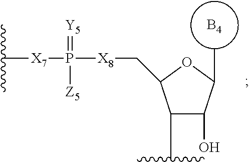
ring B₁ is guanine, a modified guanine or a guanine analog;
each of R₁ through R₄ is independently H, OH, SH, NH₂ or halogen; or one of R₁ through R₄ is selected from the group consisting of CH₃, CH₂(OH), CH(OH)₂, CH₂(SH), CH(SH)₂, CH₂(NH₂), CH(NH₂)₂, CH₂(halogen), CH(halogen)₂, and C(halogen)₃ and each of the remaining three of R₁ through R₄ is independently H, OH, SH, NH₂ or halogen;
n₁ and n₂ are each independently selected from an integer ranging from 0 to 10;
n₃ is 0, 1, 2;
L is selected from the group consisting of CH₂, O, S, SO, SO₂, N, CH(OH), CH(SH), and CH(halogen) with the proviso that, if (i) n₁ is 0 and/or (ii) n₂ is 0 and X₁ is not CH₂, L is selected from the group consisting of CH₂, CH(OH), CH(SH) and CH(halogen);
each of X₁ through X₈ is independently O, S, NH or CH₂;
each of Y₁ through Y₅ is independently O, S or Se;
each of Z₁ through Z₅ is independently OH, SH or BH₃;
R₆ is (i) selected from the group consisting of H, OH, OC₁-C₃-alkyl, and Opropargyl, wherein the dashed methylene bridge between R₆ and the 4′ C is absent, or (ii) 0, wherein the dashed methylene bridge between R₆ being 0 and the 4′ C is present;
R₈ is (i) selected from the group consisting of H, OH, OC₁-C₃-alkyl, and Opropargyl, wherein the dashed methylene bridge between R₈ and the 4′ C is absent, or (ii) O, wherein the dashed methylene bridge between R₈ being O and the 4′ C is present; and
each of ring B₂ through ring B₄ is independently a nucleobase, a modified nucleobase or a nucleobase analog;
wherein the process comprises reacting
a compound of formula (IV) 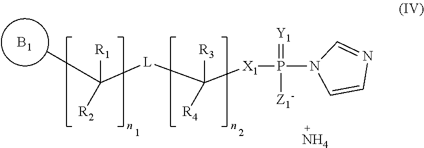
wherein B₁, R₁, R₂, R₃, R₄, n₁, n₂, X₁, Y₁, Z₁ are as defined above for formula (I); with
a compound of formula NV) 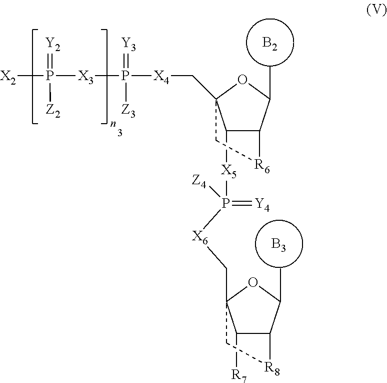
wherein X₂, Y₂, Z₂, X₃, Y₃, Z₃, X₄, Y₄, Z₄, X₅, X₆, R₆, R₇, R₈, B₂, and B₃ are as defined above for formula (I).

69. The process according to claim 68, wherein the reaction is performed in the presence of a metal chloride, preferably zinc chloride, manganese chloride or magnesium chloride, more preferably magnesium chloride.

70. The process according to any one of claim 68 or 69, wherein the reaction is performed in an aqueous solution and/or an organic solvent, preferably a mixture of water and acetonitrile or a mixture of water and N-methylmorpholine.

71. The process according to any one of claims 68 to 70, wherein the product is purified by desalting via reverse-phase HPLC.

72. The process according to any one of claims 68 to 71, wherein the process further comprises preparing the compound of formula (IV) comprising reacting a compound of formula (VI) 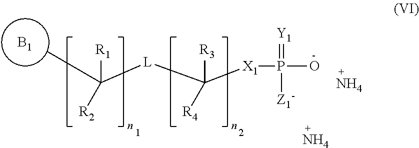
wherein B₁, R₁, R₂, R₃, R₄, n₁, n₂, X₁, Y₁, Z₁ are as defined above for formula (IV);
with carbonyldiimidazole.

73. The process according to claim 72, wherein the reaction of a compound of formula (VI) with carbonyldiimidazole is performed in DMSO.

74. The process according to any one of claims 72 and 73, wherein excess carbonyldiimidazole is quenched with water.


SEQUENCE LISTING (1 sequence)

SEQ ID NO: 1 - RNA, 1925 nt, synthetic construct
  misc_feature 1..1925: note = luciferase mRNA
  source 1..1925: mol_type = other RNA
gggagagtcccgcagtcggcgtccagcggctctgcttgttcgtgtgtgtgtcgttgcaggccttattcaagcttaccatggaggacgccaagaacatcaagaagggccccgccccgttctaccccctggaggacgggaccgcgggcgagcagctccacaaggccatgaagcggtacgccctggtgcccgggaccatcgccttcacggacgcccacatcgaggtcgacatcacctacgcggagtacttcgagatgagcgtgcgcctggccgaggccatgaagcggtacggcctcaacaccaaccaccgcatcgtggtctgctccgagaacagcctgcagttcttcatgcccgtgctgggggccctcttcatcggcgtggcggtcgccccggccaacgacatctacaacgagcgggagctgctgaactccatgggcatcagccagcccaccgtggtgttcgtctccaagaaggggctccagaagatcctgaacgtgcagaagaagctgccgatcatccagaagatcatcatcatggacagcaagacggactaccagggcttccagtccatgtataccttcgtgaccagccacctccccccggggttcaacgagtacgacttcgtccccgagtccttcgaccgcgacaagaccatcgccctgatcatgaacagctccggcagcacggggctgcccaagggcgtggccctcccccaccggaccgcgtgcgtgcgcttctcccacgcccgggacccgatcttcggcaaccagatcatccccgacaccgccatcctgagcgtcgtgcccttccaccacgggttcggcatgttcaccacgctggggtacctcatctgcggcttccgcgtggtcctgatgtaccggttcgaggaggagctgttcctccgctccctgcaggactacaagatccagagcgccctgctcgtgcccaccctgttctccttcttcgccaagagcaccctgatcgacaagtacgacctctccaacctgcacgagatcgcgagcggcggggccccgctgagcaaggaggtgggcgaggccgtcgccaagcggttccacctccccgggatccgccagggctacgggctgaccgagacgacctccgccatcctgatcacccccgagggcgacgacaagcccggcgcggtggggaaggtggtcccgttcttcgaggccaaggtggtcgacctcgacaccggcaagacgctgggggtgaaccagcggggcgagctgtgcgtgcgcgggcccatgatcatgagcggctacgtcaacaaccccgaggccaccaacgccctcatcgacaaggacggctggctgcactccggggacatcgcctactgggacgaggacgagcacttcttcatcgtggaccggctcaagagcctcatcaagtacaagggctaccaggtggcgcccgccgagctggagtccatcctgctccagcacccgaacatcttcgacgccggggtcgccggcctgcccgacgacgacgcgggggagctgcccgccgccgtggtggtcctcgagcacggcaagaccatgaccgagaaggagatcgtggactacgtggccagccaggtcacgaccgccaagaagctgcgcggcggggtggtgttcgtcgacgaggtgcccaagggcctgaccgggaagctggacgcgcggaagatccgcgagatcctcatcaaggccaagaagggcggcaagatcgccgtctgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa